(19) United States
(12) Patent Application Publication
(10) Pub. No.: US 2019/0284640 A1
(43) Pub. Date: Sep. 19, 2019

(54) Methods and Systems for Predicting Response to Immunotherapies for Treatment of Cancer

(71) Applicant: Vanderbilt University, Nashville, TN (US)

(72) Inventors: Justin M. Balko, Brentwood, TN (US); Douglas B. Johnson, Nashville, TN (US); Violeta Sanchez de Delgado, Nashville, TN (US); Melinda Sanders, Nashville, TN (US)

(21) Appl. No.: 16/355,513

(22) Filed: Mar. 15, 2019

Related U.S. Application Data
(60) Provisional application No. 62/643,599, filed on Mar. 15, 2018.

(51) Int. Cl. C12Q 1/6886; A61K 39/395; A61K 31/4523; A61K 31/40; C07K 16/28; G01N 33/574

(52) U.S. Cl. CPC C12Q 1/6886; A61K 39/3955; A61K 31/4523; A61K 31/40; C12Q 2600/106; G01N 33/5743; C07K 2317/24; C12Q 2600/158; C07K 16/2827

(57) ABSTRACT

The presently-disclosed subject matter relates to methods and systems for examining tumor samples, methods and systems for identifying subjects who are likely responders to treatment, and methods for treating cancer. In some embodiments, the presently-disclosed subject matter relates to determining expression of a major histocompatibility complex-II (MHC-II) molecule on a cell from a tumor sample, and further involving determining presence of tumor-infiltrating T cells in the tumor sample, determining the presence of tumor-infiltrating lymphocytes in the tumor sample, detecting chemokine expression in the tumor sample, and/or detecting TP53 mutations in the tumor sample. In some embodiments, the method involves treatment with an immunotherapeutic agent either alone or in combination with an MDM2 antagonist or an MEK inhibitor.

### RELATED APPLICATIONS

This application U.S. Provisional Application Ser. No. 62/643,599 filed Mar. 15, 2018, the entire disclosures of which are incorporated herein by this reference.

### GOVERNMENT INTEREST

This invention was made with government support under P50 CA98131 awarded by the National Institutes of Health. The government has certain rights in the invention.

### TECHNICAL FIELD

The presently-disclosed subject matter relates to methods and systems for examining tumor samples, methods and systems for identifying subjects who are likely responders to therapy, and methods for treating cancer. In some embodiments, the presently-disclosed subject matter relates to determining expression of a major histocompatibility complex-II (MHC-II) molecule on a cell from a tumor sample, and further involving determining presence of tumor-infiltrating T cells in the tumor sample, determining the presence of tumor-infiltrating lymphocytes in the tumor sample, detecting chemokine expression in the tumor sample, and/or detecting TP53 mutations in the tumor sample. In some embodiments, the method involves treatment with an immunotherapeutic agent either alone or in combination with an MDM2 antagonist or an MEK inhibitor.

### BACKGROUND

Immunotherapies that have been approved over the last several years have shown success in treatment of cancer; however, they are costly, they can result in patient toxicity, and they do not benefit all subjects. For example, about 20-50% of melanoma and lung cancers will respond significantly to immunotherapies, while others will not. Thus, identifying which subjects are better candidates for immunotherapy is highly advantageous from a health care and patient quality of life perspective.

PD-L1 is a cell surface glycoprotein that is one of two known ligands for Programmed Death 1 (PD-1). Expression of PD-L1 has been observed on the surface of a variety of immune cells, and PD-L1 mRNA is expressed by non- lymphoid tissues including vascular endothelial cells, epithelial cells, muscle cells, and in tonsil and placental tissue. PD-L1 expression has also been observed in a variety of human cancers, and interaction of tumor-cell expressed PD-L 1 with PD-1 can induce inhibition or apoptosis of tumor-specific T cells. In large sample sets of e.g. ovarian, renal, colorectal, pancreatic, liver cancers and melanoma it has been shown that PD-L1 expression correlated with poor prognosis and reduced overall survival irrespective of subsequent treatment. Anti-PD-1 monoclonal antibodies (mAbs) that block binding of PD-L1 to PD-1 have been shown to have anti-tumor activity against a variety of tumor types, with early human clinical data suggesting that patients whose tumors express PD-L1 are more likely to respond to anti-PD-1 therapy. See International Patent Application Publication No. WO 2014/165422.

Although immunostaining for PD-L1 on tumor cells has been reported to be associated with response in clinical trials, the staining protocol often requires frozen tissue, rather than the formalin-fixed industry standard, and is subject to technical difficulties. Further, the overall accuracy of PD-L1 staining was only 62% in a clinical study (NEJM, PMID:22658127), with imperfect negative and positive predictive value (JCO, PMID:24145345).

Accordingly, there remains a need in the art for a methods and systems for predicting response to immunotherapies, which have improved accuracy for independent use or use in tandem with existing predictive methods, such as PD-L1 staining. There also remains a need in the art for improved methods for examining tumor samples, and improved methods and compositions for treating cancer.

Immune checkpoint inhibitors that block the interaction between programmed death-1 (PD-1) and its ligand (PD-L1) have transformed the treatment landscape of numerous solid tumors (1*). These agents unleash restrained preexisting antitumor immune responses, leading to durable disease control in a substantial fraction of treated patients. Despite these advances, intrinsic and acquired resistance curtails clinical benefits in most patients.

MHC-II expression on tumor cells represents an autonomous phenotype that is associated with enhanced response to PD-1-targeted immunotherapy (12*), a finding subsequently validated in other tumor types (13*) and with combination immunotherapy (14*). While MHC-II expression is not required for response to immunotherapy in melanoma, tumors demonstrating this phenotype have particularly frequent and profound clinical responses (12*).

Molecular drivers of therapeutic resistance are incompletely characterized. Described resistance mechanisms include downregulation of antigen machinery by somatic mutations in JAK/STAT pathways (2*, 3*), alternative immune checkpoint expression (4*), loss or lack of immunogenic neoantigens (5*-10*), and tumor-intrinsic gene expression programs involving angiogenesis and wound healing (11*). Identifying effective therapeutic strategies to overcome mechanisms of resistance and characterizing novel drivers remain critical unmet needs.

### SUMMARY

The presently-disclosed subject matter meets some or all of the above-identified needs, as will become evident to those of ordinary skill in the art after a study of information provided in this document.

This Summary lists several embodiments of the presently disclosed subject matter, and in many cases lists variations and permutations of these embodiments. This

Summary is merely exemplary of the numerous and varied embodiments. Mention of one or more representative features of a given embodiment is likewise exemplary. Such an embodiment can typically exist with or without the feature(s) mentioned, likewise, those features can be applied to other embodiments of the presently disclosed subject matter, whether listed in this Summary or not. To avoid excessive repetition, this Summary does not list or suggest all possible combinations of such features.

In some embodiments, a method of examining a tumor sample from a subject involves (a) detecting cell membrane expression of a MEW molecule on a cell from the tumor sample; and (b) conducting one or more of steps (i)-(iv), including (i) determining the presence of tumor-infiltrating T cells in the tumor sample; (ii) determining the presence of tumor-infiltrating lymphocytes in the tumor sample; (iii) detecting chemokine expression in the tumor sample; and (iv) detecting TP53 mutations in the tumor sample.

In some embodiments of the methods, the MHC molecule is selected from HLA-A, HLA-B, HLA-C, HLA-DO, HLA-DM, HLA-DR, HLA-DP, HLA-DQ, and HLA-DX. In some embodiments, the cell membrane expression of the MEW molecule is detected using at least one method selected from the group consisting of immunohistochemistry, immunofluorescence, flow cytometry, mass-spectroscopy, RNA sequencing, RNA in situ hybridization, polymerase chain reaction (PCR), enzyme-linked immunosorbent assay (ELISA), and combinations thereof. In some embodiments, the cell membrane expression of the MHC molecule is detected by contacting the cell with an antibody targeting the MHC molecule and detecting binding between the MHC molecule and the antibody.

In some embodiments of the methods, the T cells are selected from CD4⁺ and CD8⁺ T cells. In some embodiments, the presence of tumor-infiltrating T cells in the tumor sample is detected using at least one method selected from the group consisting of immunohistochemistry, immunofluorescence, flow cytometry, mass-spectroscopy, RNA sequencing, RNA in situ hybridization, polymerase chain reaction (PCR), enzyme-linked immunosorbent assay (ELISA), and combinations thereof.

In some embodiments of the method, the presence of tumor-infiltrating lymphocytes in the tumor sample is detected using Haemotoxylin and Eosin staining.

In some embodiments of the method, the chemokines are selected from the group consisting of CCL5, CXCL9, CXCL10, and CXCL11. In some embodiments, the expression of chemokine expression in the tumor sample is detected using at least one method selected from the group consisting of immunohistochemistry, immunofluorescence, flow cytometry, mass-spectroscopy, RNA sequencing, RNA in situ hybridization, polymerase chain reaction (PCR), enzyme-linked immunosorbent assay (ELISA), and combinations thereof.

In some embodiments of the method, the TP53 mutations are detected by direct sequencing.

In a further embodiment, the method further includes detecting expression of a marker selected from the group consisting of: HLA-A, HLA-B, HLA-C, PD-1, PD-L1, CD8, CD4, CIITA, Foxp3, LAG3, TIM3, Ox40, Ox40L, 41BB, VISTA, Interferon gamma, Granzyme B, CTLA-4, and SOX-10. In one embodiment, the method of detecting cell membrane expression of an MHC molecule in a subject further includes staining for a cancer-specific marker. In another embodiment, the cancer-specific marker is a melanoma-specific marker, such as SOX-10.

The presently-disclosed subject matter further includes a method of identifying a subject as a likely responder to treatment with an immunotherapeutic agent when cell membrane expression of the MHC molecule on the cell is elevated, and at least one of (i)-(iv) is present: (i) a presence of tumor-infiltrating T cells in the tumor sample; (ii) a presence of tumor-infiltrating lymphocytes in the tumor sample; (iii) elevated chemokine expression in the tumor sample; and (iv) the subject has a TP53-mutation.

In some embodiments, the method also involves administering a therapeutically effective amount of an immunotherapeutic agent to the subject. In some embodiments, the immunotherapeutic agent is an antibody or an antigen-binding portion thereof that disrupts the interaction between PD-1 and PD-L1. In some embodiments, the immunotherapeutic agent is an antibody selected from anti-CTLA-4, anti-PD-L1, anti-PD-1, anti-LAG3, anti-TIM3, anti-OX40, anti-4-1BB, or an antigen-binding portion thereof. In some embodiments, the immunotherapeutic agent is administered in combination with an MDM2 antagonist or an MEK inhibitor. In some embodiments, the method involves administering a combination of an immunotherapeutic agent and a MEK, epigenetic DNA methyltransferase, or histone deacetylase inhibitor. In some embodiments, the method involves administering a combination of an anti-PD-L1 antibody and an MDM2 antagonist or an MEK inhibitor. In some embodiments, the method involves administering a combination of Atezolizumab and Cobimetinib. In some embodiments, the method involves administering a combination of comprises Atezolizumab and Idasanutlin.

In some embodiments, a method is provided where the tumor sample is collected at a first time point, and a second tumor sample is collected at a second time point, and further involves (a) detecting cell membrane expression of a MHC molecule on a cell from the second tumor sample; and (b) conducting one or more of steps (i)-(iii), including (i) determining the presence of tumor-infiltrating T cells in the second tumor sample; (ii) determining the presence of tumor-infiltrating lymphocytes in the second tumor sample; (iii) detecting chemokine expression in the second tumor sample; and (iv) detecting TP53 mutations in the second tumor sample. In some embodiments, the method also involves calculating differences between the tumor sample and the second tumor sample, including: differences in cell membrane expression levels of the MHC molecule; differences in tumor-infiltrating T cell levels; differences in tumor-infiltrating lymphocyte levels; and differences in chemokine expression levels. In some embodiments, the method and calculated differences can be used to assess response to treatment.

In some embodiments, the tumor sample is from a cancer selected from: melanoma, lung, ovarian, renal, colorectal, head and neck, bladder, endometrial, pancreatic, breast, and liver cancer, leukemia, and lymphoma. In some embodiments, the tumor sample is from a metastatic ER+ breast cancer. In some embodiments, the tumor sample is formalin-fixed. In some embodiments, the tumor sample is not a frozen tissue sample.

### BRIEF DESCRIPTION OF THE DRAWINGS

Illustrative aspects of embodiments of the present invention will be described in detail with reference to the following figures wherein:

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIGS. 1A-C A unique subtype of melanoma expresses MHC-II. (A) Microarray data from 60 melanoma cell lines in the CCLE48 were analyzed for MHC-I (HLA-A/B/C) and MHC-II (HLA-DRA) expression. Bars represent the mean ±S.D. P-value is the result of the Kolmogorov-Smirnov test comparing the distribution of MHC-I (HLA-A, HLA-B, HLA-C) expression with MHC-II expression (HLA-DRA). *represents the cutoff for defining MHC-II(+). (B) Gene-expression data from HLA-DRA(+) cell lines (Clusters Ia/Ib) were compared with HLA-DRA(−) cell lines (Clusters II and III) by an FDR-corrected row t-test. Significantly altered genes are shown on the y-axis and also listed in Supplementary Data 1 (Table 1). An ad hoc heat map is shown at the top, highlighting classical MHC-II genes. (C) Normalized microarray data were analyzed by GSA47 using the curated Molecular Signatures Database, and the resulting gene set scores are presented as a hierarchical clustered heat map.

FIG. 2 shows a graph illustrating that HLA-DR(+) melanoma cell lines are associated with a higher mutational burden. CCLE melanoma cell lines (n=61) plotted against total expressed mutational burden. Number of non-synonymous mutations was determined by targeted sequencing of 1561 genes and these data and associated information are available in the CCLE project through cBio portal (cbioportal.org).

FIGS. 3A-F show characterization of MHC-II(+) melanoma cell lines. Melanoma cell lines were treated with IFNy for 24 h before collection and live-cell staining and flow cytometry analysis for (A) MHC-I (HLA-A/B/C), (B) MHC-II (HLA-DR), and (C) PD-L1. Bars represent mean ±S.E.M. for at least three experiments (D) Representative flow plots from (C). (E) Western blot analysis of melanoma cell lines after 24 or 48 h of IFNy stimulation. (F) Phosphorylation of STAT1 (top row) and STATS (bottom row) in melanoma cell lines at 15 min after IFNy stimulation. Histograms were colored according to the arcsinh transformed ratio or MFI medians relative to the table minimum value.

FIGS. 4A-D show graphs illustrating mean expression levels of MHC-I, MHC-II, and PD-L1 at baseline and after IFNy stimulation. Melanoma cell lines were treated with 100 ng IFNy for 24 hr (shown as percentage positive in FIGS. 2A-C) prior to harvest and live-cell staining and flow cytometry analysis for (A) MHC-FHLA-A/B/C, (B) MHC-II/HLA-DR, and (C) PD-L1. Bars represent mean fluorescence intensity ±SEM for 3 experiments. (D) Histograms of HLA-DR surface expression over an extended (24-72 hr) IFNy treatment, as assessed by flow cytometry.

FIGS. 5A-D show MHC-II-positive melanoma cell lines associate with NRAS mutations. (A) HLA-DRA mRNA expression in melanoma cell lines (n=60; one cell line lacked mRNA expression data) from the CCLE compared by genotype. P value (P<0.05) represents result of Tukey's post hoc analysis comparing pan-WT with NRAS-mutant cell lines, following a significant ANOVA (P=0.03) performed among all groups. Bars represent mean±S.E.M. (B) Representative IHC for HLA-DR (brown) and SOX10 (pink) in cases with isolated stromal positivity (top) and with tumor-specific staining (bottom). Both HLA-DR and SOX10 immunostaining is present in all four sections. Scale bar, 50 μm. (C) Analysis of HLA-DR IHC in a melanoma TMA (n=67 evaluable) by genotype. P value represents result of a χ²-test. (D) Overall survival of patients (n=58 evaluable) within the TMA by HLA-DR status (left censored at time of diagnosis). The remaining patient samples were included from outside institutions and follow-up data were not available from those institutions. P value is the result of the log-rank test.

FIGS. 6A-D show Ex vivo culture of tumors derived from anti-PD-1-responding and non-responding patients identifies heterogeneity in interferon response. (A) Patient tumor blocks stained for HLA-DR (brown) and SOX10 (pink) at low (scale bar, 500 um) and high magnification (scale bar, 200 gm); PT1: anti-PD-1 non-responder and PT2: anti-PD-1 responder. (B) Experimental schema. (C) Schema and images of PDX tissue sections (ex vivo organotypic culture). (D) Western blot analysis of tissue sections cultured in the presence or absence of IFNy for 24-48 h.

FIGS. 7A-E show MHC-II(+) melanomas have improved response rates and clinical benefit to PD-1/PD-1.1 inhibition. (A) HLA-DR positivity by IHC plotted versus response to PD-1/PD-L1-targeted therapy in the discovery set (n=30). Responders include partial and complete responders; non-responders include mixed responders and progressive disease patients. Mixed responders (n=3) are noted by a red triangle. P value is the result of the Wilcoxon's rank sum test. (B) HLA-DR positivity by IHC in the validation set (n=23) plotted versus response to PD-1/PD-L1-targeted therapy. P value is the result of the Wilcoxon's rank sum test (C) Representative images of scans from anti-PD-1 therapy-treated MHC-II(+) patients (D) Progression-free survival (left) and overall survival (right) in anti-PD-1/PD-L1-treated patients, stratified by HLA-DR/MHC-II positivity (5% total tumor cells staining on entire tissue section used as cut point). Data from both the initial and validation cohorts were included, when available. P value is the result of the log-rank test. (E) Correlation matrix of IHC markers. P values for the Pearson's correlation appear above the diagonal and correlation coefficients (r) appear below the diagonal.

FIG. 8 shows graphs illustrating progression-free and overall survival as a function of MHC-II positivity cut-points. Statistical significance of PFS (top) and OS (bottom) were assessed by the log-rank statistic using different cut-points for HLA-DR positivity (5%, 10%, and 20% of tumor cells positive).

FIG. 9 shows a graph illustrating that MHC-II/HLA-DR positivity is not associated with ipilumumab response. Tumor membrane-specific HLA-DR expression quantified by IHC in excisional samples from patients (n=13) treated with ipilumumab (after tissue collection) were compared to treatment response. P-value represents the result of the Wilcoxan rank sum test for all responder groups versus non-responders (PD).

FIGS. 10A-D show graphs illustrating high correlation of staining for two independent monoclonal antibodies for MHC-II in melanomas. (A) forty-one (41) melanoma sections were co-stained for HLA-DR and SOX10 or HLA/DR/DP/DQ/DX and SOX10 and percent of tumor cells in the entire section with MHC-II(+) membranes were calculated. There was a high degree of concordance between staining for the two antibodies. There are 21 data points at (0,0). (B) HLA-DR/DP/DQ/DX positivity was used to test for association with clinical response as described for FIGS. 7A-B. P-value is the result of the Wilcoxan rank sum test. (C) and (D) PFS and OS respectively, in 26 patients (Discovery set only, non-evaluable stains excluded) discriminated on MHC-II (HLA-DR/DP/DQ/DX positivity, using a 5% cut-point (5% of total tumor cells staining positive on the entire section; no tumors stained between 1-5%). P-value represents the result of the log-rank test.

FIG. 11 shows a graph illustrating that MHC-FHLA-A positivity is not associated with PD-1/PD-L1 targeted therapy response. Tumor membrane-specific HLA-A expression quantified by IHC in excisional samples from patients treated with PD-1/PD-L1 targeted therapy (after tissue collection) is compared to treatment response. P-value represents the result of the Wilcoxan rank sum test for all responder groups versus PD. Mixed responders (n=3) are noted by a red triangle.

FIG. 12 shows graphs illustrating that CD4 positivity is not associated with PD-1/PD-L1 targeted therapy response. Tumor-infiltrating CD4(+) (left) and CD8(+) (right) cells quantified by IHC in excisional samples from patients treated with PD-1/PD-L1 targeted therapy (after tissue collection) is compared to treatment response. P-values are result of a Wilcoxan rank sum test for all responder groups versus PD. Mixed responders (n=3) are noted by a red triangle.

FIGS. 13A-C show graphs and images illustrating a lack of PD-L1 staining with response to PD-1/PD-L1 targeted therapy. (A) representative immunostaining for SOX10 (brown/DAB) and PD-L1 (pink/Warp Red) in human placenta (positive control), a PD-L1(−) tumor, and two PD-L1(+) tumors. (B) lack of association of PD-L1 positivity with response in a series of 24 anti-PD-1/PD-L1-treated melanoma patients. Only 4/24 patients had PD-L1 positivity noted in the tumor compartment. (C) lack of correlation between tumor cell positivity of PD-L1 and HLA-DR by IHC staining.

FIGS. 14A-B show graphs illustrating that constitutive expression of MHC-II is selected against in B16 cells, but may have a functional role in response to anti-PD-L1 targeted therapy. (A) flow cytometry sorting of B16/F0 melanoma cells (anti-IA/IE) after lentiviral transduction with mouse Ciita. LACZ was used as a control for lentiviral transduction. After sorting, the percent of MHC-II+ cells was rapidly selected against in culture, despite negative selection with puromycin. (B) lentivirally-transduced cells (50,000 LACZ or Ciita) were injected subcutaneously into the flanks of C57/BL6 mice, which were subsequently treated twice weekly with 100 μg/100 μL anti-mouse PD-L1 mAB (BioXcel) intraperitoneally beginning on day 1 after tumor challenge. For tumor challenge, 3 separate experiments were performed for Ciita+ cell injections (assessed by flow cytometry at the day of injection as containing 10, 20, or ˜30% MHC-II/IA/IE+ cells). Tumor volume was measured thrice weekly. Survival curves combined all cohorts of Ciita+ injected mice. Tumor ulceration or tumors exceeding 1000 mm^{#} was used as an endpoint for survival.

FIGS. 15A-B show images illustrating membrane staining of (A) non-responders and (B) responders.

FIG. 16 shows a graph illustrating patient classification by clinical response to targeted immunotherapy. PR and CR refer to partial and complete response, and PD refers to progressive disease.

FIG. 17 shows MHC-II/HLA-DR expression in patient tumor samples is associated with unique patterns of inflammation and enhanced CD4, CD8, and LAG-3+ infiltrate. (A) Representative images of IHC from HLA-DR+ and HLA-DR− tumors. HLA-DR is stained in brown (DAB), and Sox10, a nuclear melanoma marker, is stained in pink (Mach Red). Scale bar: 50 (B) Gene set analysis from RNA-sequencing analysis of IHC-defined tumor HLA-DR+ (>5% tumor cells) or HLA-DR− (<5% tumor cells) melanoma and lung specimens. After significant (FDR <10%) gene set scores were defined, scores were created as the mean of all genes in each signature for each sample and plotted as row-standardized Z-scores with heatmap representation. (C) Overlap of enriched gene sets in MHC-II+ tumors versus melanoma cell lines (ref. 12*). (D) Pearson's correlation matrix of gene expression associations between immune response and inhibitory markers among PD-1-treated patient samples from melanoma and lung cancer (n=50). Data represent correlation among TPM RNA-sequencing values, except “HLA-DR TUMOR,” which is the correlation with tumor HLA-DR percent positivity by IHC (n=41 of 50 available data points). Values in the individual boxes represent the Pearson's correlation coefficient.

FIG. 18 shows MHC-II/HLA-DR expression in patient tumor samples is associated with LAG-3+ infiltrate. (A) RNA-sequencing expression levels of checkpoint and checkpoint ligands by HLA-DR status of the tumor. n=41; *P<0.05; **P<0.01, 2-tailed t test. (B) RNA-sequencing expression levels of checkpoint and checkpoint ligands by patient immune-related response criteria. PD, progressive disease; SD/MR, stable disease or mixed response; PR, partial response; CR, complete response; RELAPSE, sample collected at relapse/progression after initial PR/CR. n=57; *P<0.05, Tukey's post hoc test. (C) RNA-sequencing expression levels of checkpoints in 3 pairs of matched preresponse and postrelapse specimens. P value represents paired 2-tailed t test. (D) Representative IHC for LAG-3 in a melanoma sample before anti-PD-1 response and at progression. Scale bar: 50 (E) IHC analysis for LAG-3⁺ TILs in 6 paired melanoma specimens before anti-PD-1 response and at progression.

FIG. 19 shows MHC-II+ breast tumors recruit CD4+ T cells and are associated with Lag-3+ TILs. (A) Representative positive control for Lag-3 IHC in human tonsil. Scale bar: (B) Representative Lag-3⁺ breast cancer TILs by IHC. Arrows indicate Lag-3+ TILs. Scale bar: 50 (C) Representative AQUA immunofluorescence image of a HLA-DR⁺ breast tumor case. Scale bar: 100 μm. (D) Difference in tumor-specific HLA-DR expression among LAG-3⁺ and LAG-3⁻ infiltrated triple-negative breast cancers in all patients (n=86; left) and only those patients with a percentage of stromal TILs >20% (n=31; right). ***P<0.001, 2-tailed t test. (E) CD4 and CD8 and the difference between CD4 and CD8 infiltration in HLA-DR-high (HI) and HLA-DR-low (LO) expressing tumors. **P<0.01, 2-tailed t test. (F) Association of stromal (noncytokeratin⁺) PD-L1 expression with HLA-DR expression. **P<0.01, 2-tailed t test.

FIG. 20 shows MHC-II expression in murine mammary carcinoma potentiates antitumor immunity. (A) Flow cytometry of MMTV-neu cells transduced with Ciita or vector control, stained with anti-MHC-II-Alexa Fluor 488 (IA-IE) or isotype control. (B) Tumor growth or orthotopic MMTV-neu cells in wild-type syngeneic FVB/n mice. Red lines represent rejected engraftments. (C) Proportions of rejected tumor engraftments for control or Ciita⁻ MMTV-neu cells. P value represents Fisher's exact test. (D) Tumors (n=12) from B that were not rejected were examined by H&E for TILs and T cell compartments by IHC (CD8, CD4, and Foxp3) and scored as a percentage of total TILs. P values represent 2-tailed Mann-Whitney test.

FIG. 21 shows Enhanced T cell-recruiting chemokines and Lag3 expression in MHC-II+ murine tumors. (A) Heatmap visualization of altered gene expression levels in nonrejected tumors from FIG. 18H. Transcript counts (NanoString PanCancer Immune profiling) were row normalized/standardized for visualization. (B) Gene expression levels of immune checkpoints Pd-1, Tim-3, and Lag-3 in Ciita⁺ and control tumors (n=20). (C) Cultured MMTV-neu tumor cells (cell line) expressing Ciita or vector control were analyzed by quantitative real-time PCR for tumor-specific changes in T cell-recruiting chemokines. n=3 independent experiments. *P<0.05; **P<0.01; ***P<0.001, a 2-tailed t test. (D) NanoString gene expression data from MMTV-neu Ciita⁺ or vector control tumors (n=12) harvested at humane endpoints (1.5-2 cm³) were queried for expression levels of T cell-attracting chemokines (see FIG. 34A for eomesodermin [Eomes]). *P<0.05; **P<0.01, 2-tailed t test.

FIG. 22 shows (A) RNA-sequencing gene expression data from HLA-DR⁺ and HLA-DR⁻ tumors (n=41) were analyzed for differences in the human orthologs for chemokines in FIG. 21. *P<0.05, **P<0.01, by Mann Whitney U test. (B) TCGA breast cancer data set was utilized to explore correlations between HLA-DRA mRNA and T cell chemokines across over 1000 patients. A correlation matrix was generated showing the Pearson's correlation coefficient across each gene pair.

FIG. 23 shows (A) Schematic of experimental strategy. On day −10, 1×10⁶ MMTV-neu cells transduced with pMX-puro or pMX-Ciita were implanted by orthotopic injection in the 4th mammary fat pad of wild-type FVB/n mice. Seven days later, a subset of mice were sacrificed for flow cytometry analysis. On day 0, therapy was initiated consisting of twice weekly i.p. injections of anti-IgG control, anti-PD-1, or anti-PD-ranti-Lag-3, which continued for 2 weeks. Tumors were monitored for growth over the next 39 days. (B) Schematic for analyzing tumor and lymphoid compartments by flow cytometry. (C) Tumor growth curves for treated mice. CR, complete response. **P<0.05, χ² test across treatment groups in Ciita-expressing tumors. (D) Flow cytometry analysis of PD-1⁺/Lag-3⁺ lymphocytes by total CD3⁺ compartment or by CD3⁺/CD4⁺ or CD3⁺ CD8⁺ compartments in lymphoid tissues at 7 days after injection. **P<0.01, 2-tailed Mann Whitney U test. (E) Flow cytometry analysis of PD-1⁺/Lag-3⁺ lymphocytes in tumors (TILs). *P<0.05, Mann Whitney U test.

FIG. 24 shows the alternative MHC-II receptor FCRL6 is an inhibitor of T cell and NK cell activity. (A) NK-92 and K562 transductants were stained with anti-FCRL6 or anti-HLA-DR, respectively. K562 vector (top), K562 DRaTh1 (middle), or K562 CIITA (bottom) transductants were cultured with NK-92 FCRL6 transductants (red lines), NK-92 vector (blue lines), or parental NK-92 cells (black lines) at various effector-to-target (E/T) ratios and assayed for K562 cytotoxicity in ⁵¹Cr release assays. Experiments were performed in triplicate; lines represent the mean±SD. n=4; P values were calculated using Student's t test. *P<0.01, #P<0.02. (B) Blood mononuclear cells were cultured for 6 days in the presence of the CEF peptide pool with anti-FCRL6, anti-PD-L1, or an isotype-matched control mAb. Cells were collected, restimulated with CEF for 6 hours, and analyzed for intracellular cytokine production by flow cytometry. CD8⁺ T cells from one representative donor were gated, and the percentages of cells staining positive for the indicated cytokines are shown in the quadrants of each dot plot. (C) Paired data points from healthy donors (n=12 for IFN-γ; n=9 for TNF-α) are indicated by lines, and statistical significance was determined using the Wilcoxon signed-rank test.

FIG. 25 shows The alternative MHC-II receptor FCRL6 is enriched in MHC-II+ tumors and is associated with acquired resistance to anti-PD-1.(A) mRNA expression from RNA-sequencing data for FCRL3 and FCRL6 in MHC-It and MHC-II⁻ melanoma and lung cancers. (B) mRNA expression from RNA-sequencing data for FCRL3 and FCRL6 in untreated and postprogression/relapse specimens. (C) Quantification of IHC for FCRL6⁺ TILs in pairs of melanomas before and after resistance to anti-PD-1 therapy. (D) MHC-II/HLA-DR AQUA scores in triple-negative breast cancers stratified by the presence or absence of FCRL6⁺ lymphocytes. (E) Coexpression of Lag-3 and FCRL6 on TILs in postneoadjuvant triple-negative breast cancers. (F) MHC-II/HLA-DR AQUA scores in triple-negative breast cancers stratified by the presence or absence of FCRL6⁺ lymphocytes and Lag-3⁺ lymphocytes. (G) Fraction of CD8⁺ lymphocytes expressing granzyme B (AQUA), stratified by the presence or absence of FCRL6⁺ lymphocytes (IHC), Lag-nymphocytes (IHC), or total tumor microenvironment PD-L1 expression (AQUA). *P<0.05 by Mann Whitney U test.

FIG. 26 shows MHC-II/HLA-DR expression by melanoma cells colocalizes with CIITAmRNA expression. Dual RNAish (CIITA, pink) and IHC (HLA-DR, brown) colocalizes on the same cells, suggesting that MHC-II expression is endogenous and represents a tumor cell autonomous phenotype rather than a result of trogocytosis.

FIG. 27 shows MHC-II gene expression and T cell markers signatures by tumor-specific HLA-DR status (IHC). RNAseq expression levels of HLA-DRA, CD8A, CD4, and Foxp3 genes in MHC-II/HLA-DR+ tumors (>5% tumor cells positive) versus negative. *p<0.05 two-tailed t test.

FIG. 28 shows CyTOF analysis of human melanomas. (A) Two freshly resected human melanomas were dissociated and analyzed by CyTOF using an immune contexture-based mass cytometry panel. Healthy human PBMCs were utilized as a control. ViSNE analysis was used to identify T cell populations and the expression of phenotypic markers within those populations. Scaled expression is represented by each color bar specific to the depicted marker. (B) Biplots demonstrating overlapping populations of HLA-DR, LAG3, and PD-1 on T cells in the melanoma specimens from (A).

FIG. 29 shows interferon-gamma gene expression signatures by outcome in PD-1 treated patients. RNAseq expression levels of IFN-γ (A) and IFN-γ-responsive (B) gene signatures by melanoma and lung patient irRC. *p<0.05, Turkey's post-hoc test.

FIG. 30 shows MHC-I and MHC-II gene expression signatures by outcome in PD-1 treated patients. RNAseq expression levels of HLA-A (A) and HLA-DRA (B) genes by melanoma and lung patient irRC.

FIG. 31 shows correlates of LAG-3+ TILs in TNBC. Percent-tumor infiltrating lymphocytes across TNBC patients, stratified by the presence of LAG-3+ cells. *** p<0.001; Mann Whitney U test. B) Percent- PD-L1+ stroma (AQUA score) across TNBC patients, stratified by the presence of LAG-3+ cells. * p<0.05; Mann Whitney U test.

FIG. 32 shows Tumor growth rates in MMTV-neu Ciita+ and puro-control tumors. Only mice which formed tumors (did not reject) are included.

FIG. 33 shows Tumors escaping immunologic rejection in enforced- MHC-II expressing MMTV-neu tumors retain MHC-II expression. NanoString analysis of MHC-I and MHC-II gene expression in pMX-puro and pMX-Ciita transduced tumor cells from tumors harvested following tumor establishment.

FIG. 34 shows Higher eomesodermin expression in MHC-II+ tumors. A) NanoString analysis of eomesodermin gene expression in pMX-puro and pMX-Ciita transduced tumor cells from tumors harvested following tumor establishment. B) Eomesodermin gene expression as measured by RNAseq in melanoma and lung cancer patients, stratified by MHC-II status. **p<0.01; Mann Whitney U test.

FIG. 35 shows Tumors escaping immunologic rejection in enforced MHC-II-expressing MMTV-neu tumors demonstrate higher CXCR3+ infiltrates. NanoString analysis of Cxcr3 gene expression in pMX-puro and pMX-Ciita transduced tumor cells from tumors harvested following tumor establishment. ** p<0.01 two-sample t test.

FIG. 36 shows variable expression of myeloid cell chemoattractants in Ciita+ tumors. NanoString analysis of Cxcr3 gene expression in pMX-puro and pMX-Ciita transduced tumor cells from tumors harvested following tumor establishment. ** p<0.01 two-sample t test.

FIG. 37 shows Gating strategy. A) Pulse geometry for identifying/enriching single-cell lymphocytes. B) Identification of CD4/CD8+ T cells. C) Fluorescence-minus-one controls for PD-1+ CD3 cells and Lag3+ CD3 cells (Gated on CD3+ cells for FMO controls). Left-CD3+ cells with no PD-1 or Lag3 antibody. Middle—CD3+ cells with only PD-1 antibody. Right-CD3+ cells with only Lag3 antibody.

FIG. 38 shows association of MHC-II receptor gene expression with degree of MHC-II positivity on tumor cells. RNAseq TPM counts for CD233/LAG-3 and FCRL6 were regressed and correlated to the fraction of HLA-DR+ tumor cells in the same specimen.

FIG. 39 shows representative FCRL6 IHC staining. Human spleen and lymph node tissue sections were used to validate IHC staining for FCRL6, according to the reported methods.

FIG. 40 shows no association of FCRL6+ TILs and MHC-I (HLA-A) expression on tumor cells. No association was detected between the presence of FCRL6+ TILs and HLA-A expression on tumor cells (H-score; intensity [0-3+]* fraction of tumor cells staining positive).

FIG. 41 shows expansion of FCRL6+/LAG3+ CD8 T cells following TCR stimulation. A) Healthy human subject PBMCs were cultured with anti-CD3/CD28 beads for 24, 38, or 72 hours (or 72 hours without stimulation) and assayed by flow cytometry for CD8, FCRL6, PD-1, and LAG3. B) Co-expression of FCRL6 and PD-1 or FCRL6 and LAG3 on CD8 T cells in experiments from (A).

FIG. 42 shows FCRL6 and LAG3 do not colocalize with Foxp3+ regulatory T cells. No colocalization is observed by dual IHC for FCRL6 and Foxp3 or Lag3 and Foxp3 in human melanomas. While representative images are presented, a complete pathology analysis of the tissue produced no conclusively dual-positive cells.

FIG. 43 shows the association of sTILs with outcome after surgery.

FIG. 44 shows that a significant percentage of sTIL cells were positive for CD4 or CD8.

FIG. 45 shows the correlation between the association of T-cell composition with outcome.

FIG. 46 shows checkpoint TP53 mutations have distinct chemokine and immune expression signatures.

FIG. 47 shows chemokine expression derived from gene expression data using nanostring gene expression profiling.

FIG. 48 shows TP53 mutant mouse breast cancer cells lines induce cytokine expression hollowing doxorubicin treatment.

FIG. 49 shows doxorubicin induces T cell recruiting chemokines in p53 altered

MMTV-Neu cells. FIG. 49.

FIG. 50 shows mined data from the Cancer Genome Atlas (TCGA) showing higher expression of chemokines in human breast tumors with TP53 gene mutations.

FIG. 51 shows mined data from the Cancer Genome Atlas (TCGA) showing higher expression of chemokines in human breast tumors with TP53 gene mutations.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

Unless defined otherwise, all technical and scientific terms used herein have the same meaning as is commonly understood by one of skill in the art to which the invention(s) belong. All patents, patent applications, published applications and publications, GenBank sequences, databases, websites and other published materials referred to throughout the entire disclosure herein, unless noted otherwise, are incorporated by reference in their entirety. In the event that there is a plurality of definitions for terms herein, those in this section prevail. Where reference is made to a URL or other such identifier or address, it understood that such identifiers can change and particular information on the internet can come and go, but equivalent information can be found by searching the internet. Reference thereto evidences the availability and public dissemination of such information.

Although any methods, devices, and materials similar or equivalent to those described herein can be used in the practice or testing of the presently-disclosed subject matter, representative methods, devices, and materials are now described.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

Unless otherwise indicated, the term “administering” is inclusive of all means known to those of ordinary skill in the art for providing a preparation to a subject, including administration by inhalation, nasal administration, topical administration, intravaginal administration, ophthalmic administration, intraaural administration, intracerebral administration, intravitreous administration, intracameral administration, posterior sub-Tenon administration, posterior juxtascleral administration, subretinal administration, suprachoroidal administration, cell-based administration or production, rectal administration, and parenteral administration, including injectable such as intravenous administration, intra-arterial administration, intramuscular administration, and/or subcutaneous administration. Administration can be continuous or intermittent.

In some embodiments a subject will be administered an effective amount of at least one compound and/or composition provided in the present disclosure. In this respect, the term “effective amount” refers to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products.

Additionally, the terms “subject” or “subject in need thereof” refer to a target of administration, which optionally displays symptoms related to a cancer. The subject of the herein disclosed methods can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. A patient refers to a subject afflicted with a disease or disorder. The term “subject” includes human and veterinary subjects.

As will be recognized by one of ordinary skill in the art, the terms “suppression,” “suppressing,” “suppressor,” “inhibition,” “inhibiting” or “inhibitor” do not refer to a complete elimination of angiogenesis in all cases. Rather, the skilled artisan will understand that the term “suppressing” or “inhibiting” refers to a reduction or decrease in angiogenesis. Such reduction or decrease can be determined relative to a control. In some embodiments, the reduction or decrease relative to a control can be about a 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% decrease.

As used herein, the terms “treatment” or “treating” relate to any treatment of a cancer. As such, the terms treatment or treating include, but are not limited to: preventing a condition of interest or the development of a condition of interest; inhibiting the progression of a condition of interest; arresting or preventing the development of a condition of interest; reducing the severity of a condition of interest; ameliorating or relieving symptoms associated with a condition of interest; and causing a regression of the condition of interest or one or more of the symptoms associated with the condition of interest.

The presently-disclosed subject matter includes methods and systems for examining tumor samples, methods and systems for identifying subjects who are likely responders to treatment, and methods for treating cancer. In some embodiments, the presently-disclosed subject matter relates to determining expression of a major histocompatibility complex-II (MHC-II) molecule on a cell from a tumor sample, and further involving determining presence of tumor-infiltrating T cells in the tumor sample, determining the presence of tumor-infiltrating lymphocytes in the tumor sample, detecting chemokine expression in the tumor sample, and/or detecting TP53 mutations in the tumor sample. In some embodiments, the method involves treatment with an immunotherapeutic agent either alone or in combination with an MDM2 antagonist or an MEK inhibitor. Also disclosed are kits useful for treating and/or predicting whether a subject is likely to benefit from treatment with an immunotherapy.

In some embodiments, a method of examining a tumor sample from a subject involves (a) detecting cell membrane expression of a MHC molecule on a cell from the tumor sample; and (b) conducting one or more of steps (i)-(iv), including (i) determining the presence of tumor-infiltrating T cells in the tumor sample; (ii) determining the presence of tumor-infiltrating lymphocytes in the tumor sample; (iii) detecting chemokine expression in the tumor sample; and (iv) detecting TP53 mutations in the tumor sample. The method can be performed ex vivo or in vitro.

With regard to the step of detecting cell membrane expression of a MHC molecule, the expression can be determined, for example, by providing a tumor sample from the subject, and determining the level of cell membrane expression of a MHC molecule, which can be an MHC-I or MHC-II molecule. In some embodiments, the MHC molecule is selected from HLA-A, HLA-B, HLA-C, HLA-DO, HLA-DM, HLA-DR, HLA-DP, HLA-DQ, and HLA-DX. In some embodiments, multiple distinct MHC molecules, or markers, are detected. For example, in one embodiment, the MHC molecule includes HLA-DR. In another embodiment, the MHC molecule includes HLA-DR and at least one of HLA-A, HLA-B, HLA-C, PD-1, or PD-L1.

In some embodiments, the cell membrane expression of the MHC molecule is detected using at least one method selected from the group consisting of immunohistochemistry, immunofluorescence, flow cytometry, mass-spectroscopy, RNA sequencing, RNA in situ hybridization, polymerase chain reaction (PCR), enzyme-linked immunosorbent assay (ELISA), and combinations thereof. In some embodiments, the cell membrane expression of the MHC molecule is detected by contacting the cell with an antibody targeting the MHC molecule and detecting binding between the MHC molecule and the antibody.

In some embodiments of the methods, the T cells are selected from CD4⁺ and CD8⁺ T cells. In some embodiments, the presence of tumor-infiltrating T cells in the tumor sample is detected using at least one method selected from the group consisting of immunohistochemistry, immunofluorescence, flow cytometry, mass-spectroscopy, RNA sequencing, RNA in situ hybridization, polymerase chain reaction (PCR), enzyme-linked immunosorbent assay (ELISA), and combinations thereof.

In some embodiments of the method, the presence of tumor-infiltrating lymphocytes in the tumor sample is detected using Haemotoxylin and Eosin staining.

In some embodiments of the method, the chemokines are selected from the group consisting of CCL5, CXCL9, CXCL10, and CXCL11. In some embodiments, the expression of chemokine expression in the tumor sample is detected using at least one method selected from the group consisting of immunohistochemistry, immunofluorescence, flow cytometry, mass-spectroscopy, RNA sequencing, RNA in situ hybridization, polymerase chain reaction (PCR), enzyme-linked immunosorbent assay (ELISA), and combinations thereof.

In some embodiments of the method, the TP53 mutations are detected by direct sequencing.

In some embodiments of the methods disclosed herein, it can be beneficial to also probe for a cancer-specific marker, e.g., a dual stain, to facilitate identification of cancer cells in the sample. For one non-limiting example, the cancer-specific marker could be a melanoma-specific marker, such as SOX-10.

Additionally or alternatively, the markers may include, but are not limited to, CD8, CD4, CIITA, Foxp3, LAG3, TIM3, Ox40, Ox40L, 41BB, VISTA, Interferon gamma, Granzyme B, interferon gamma response gene signatures, CTLA-4, SOX-10, or a combination thereof.

In some embodiments, the methods disclosed herein involve identifying likely responders to treatment with an immunotherapy or combination therapy. For example, in some embodiments, the method involves identifying a subject as a likely responder when cell membrane expression of the MHC molecule on the cell is elevated, and at least one of (i)-(iv) is present: (i) a presence of tumor-infiltrating T cells in the tumor sample; (ii) a presence of tumor-infiltrating lymphocytes in the tumor sample; (iii) elevated chemokine expression in the tumor sample; and (iv) the subject has a TP53-mutation. Elevated levels can be determined relative to a control, or with reference to a predetermined standard.

A “predetermined standard” or “reference” can include, for example, a specific expression level threshold. In some embodiments, the reference can include control data. Control data, when used as a reference, can comprise compilations of data, such as may be contained in a table, chart, graph, e.g., standard curve, or database, which provides amounts or levels of expression considered to be threshold levels or control levels. Such data can be compiled, for example, by obtaining expression levels from one or more tumor samples (e.g., an average of amounts or levels from multiple samples) from one or more individuals with the cancer of interest or without the cancer of interest.

In some embodiments of the presently-disclosed subject matter, a likely responder/a subject can be identified as being likely to benefit from treatment is administered such treatment. In this regard, some embodiments of the methods further include administration of an immunotherapeutic agent. In some embodiments, the immunotherapeutic agent is an antibody or an antigen-binding portion thereof that disrupts the interaction between PD-1 and PD-L1. In some embodiments, the immunotherapeutic agent is an antibody selected from anti-CTLA-4, anti-PD-L1, anti-PD-1, anti-LAG3, anti-TIM3, anti-Ox40, anti-4-1BB, or an antigen-binding portion thereof.

In some embodiments, the method optionally involves administering a MEK, epigenetic DNA methyltransferase, and/or histone deacetylase inhibitor. Examples of MEK inhibitors include, but are not limited to, Selumetinib (AstraZeneca), PD0325901 (Pfizer), Pimasertib, MEK inhibitor AS703026 (Merck Serono), Cobimetinib (Exelixis), Trametinib (Mekinist), binimetinib (Array BioPharma Inc), MEK inhibitor WX-554 (Wilex), refametinib (Ardea Biosciences), and AZD8330 (AstraZeneca).

In some embodiments, the immunotherapeutic agent is administered in combination with an MDM2 antagonist or an MEK inhibitor. In some embodiments, the method involves administering a combination of an immunotherapeutic agent and a MEK, epigenetic DNA methyltransferase, or histone deacetylase inhibitor. In some embodiments, the method involves administering a combination of an anti-PD-L1 antibody and an MDM2 antagonist or an MEK inhibitor. In some embodiments, the method involves administering a combination of Atezolizumab and Cobimetinib. In some embodiments, the method involves administering a combination of comprises Atezolizumab and Idasanutlin.

In some embodiments, a method is provided where the tumor sample is collected at a first time point, and a second tumor sample is collected at a second time point, and further involves (a) detecting cell membrane expression of a MEW molecule on a cell from the second tumor sample; and (b) conducting one or more of steps (i)-(iii), including (i) determining the presence of tumor-infiltrating T cells in the second tumor sample; (ii) determining the presence of tumor-infiltrating lymphocytes in the second tumor sample; (iii) detecting chemokine expression in the second tumor sample; and (iv) detecting TP53 mutations in the second tumor sample. In some embodiments, the method also involves calculating differences between the tumor sample and the second tumor sample, including: differences in cell membrane expression levels of the MHC molecule; differences in tumor-infiltrating T cell levels; differences in tumor-infiltrating lymphocyte levels; and differences in chemokine expression levels. In some embodiments, the method and calculated differences can be used to assess response to treatment.

The presently-disclosed subject matter further includes kits useful for treating and/or predicting whether a subject is likely to benefit from treatment with an immunotherapy. In some embodiments, the kit includes a probe for a MHC molecule, selected from an MHC-I or MHC-II molecule. The kit can optionally include standards to which the level of cell membrane expression of the MHC molecule in the tissue sample from the subject is compared. In some embodiments, wherein the MHC molecule is selected from one or more of HLA-A, HLA-B, HLA-C, HLA-DA, HLA-DM, HLA-DR, HLA-DP, HLA-DQ, and HLA-DX.

The term “cancer” refers to all types of cancer or neoplasm or malignant tumors found in animals, including leukemias, carcinomas, melanoma, and sarcomas. Examples of cancers include, but are not limited to, melanoma, lung, ovarian, renal, colorectal, head and/or neck, bladder, endometrial, pancreatic, breast cancer, including metastatic ER+ breast cancer, liver cancer, leukemia, and lymphoma.

The presently-disclosed subject matter has the benefit of being useful in connection with a variety of tissue preparations. For example, while the methods disclosed herein can be used in connection with frozen tissue, frozen tissue is not required. Indeed, the methods can be used in connection with a formalin-fixed sample.

The probe included in the kit can be, for example, an antibody or an antigen-binding portion thereof that binds specifically to the cell surface-expressed MHC molecule. In some embodiments, the antibody or an antigen-binding portion thereof binds specifically to the cell surface-expressed MHC molecule in a formalin-fixed, paraffin-embedded (FFPE) tissue sample. The antibody or an antigen-binding portion can optionally include a tag, such as a fluorescent tag. In some embodiments, the kit includes a secondary antibody including a tag.

In some embodiments, the kit can optionally include a cancer-specific marker. For example, the cancer-specific marker could be a melanoma-specific marker, such as SOX-10.

In some embodiments of the kit, an immunotherapeutic agent is also provided. For example, the immunotherapeutic agent can be an antibody or an antigen-binding portion thereof that disrupts the interaction between PD-1 and PD-L1. In some embodiments, the immunotherapeutic agent is an antibody selected from anti-CTLA-4, anti-PD-L1, anti-PD-1, anti-LAG3, anti-TIM3, anti-Ox40, anti-4-1BB, or an antigen-binding portion thereof.

In some embodiments of the kit, a MEK, epigenetic DNA methyltransferase, and/or hi stone deacetylase inhibitor is also provided. Examples of MEK inhibitors include, but are not limited to, Selumetinib (AstraZeneca), PD0325901 (Pfizer), Pimasertib, MEK inhibitor AS703026 (Merck Serono), Cobimetinib (Exelixis), Trametinib (Mekinist), binimetinib (Array BioPharma Inc), MEK inhibitor WX-554 (Wilex), refametinib (Ardea Biosciences), and AZD8330 (AstraZeneca).

The presently-disclosed subject matter further includes a cell surface-expressed MHC molecule, as disclosed herein, in complex with an antibody or an antigen-binding portion thereof that binds specifically to the MHC molecule, as disclosed herein.

The presently-disclosed subject matter is further illustrated by the following specific but non-limiting examples. The following examples may include compilations of data that are representative of data gathered at various times during the course of development and experimentation related to the present invention.

### EXAMPLES

### Example 1

Melanoma-specific MHC-II expression represents a tumor-autonomous phenotype and predicts response to anti-PD-1/PD-L1 therapy.

Abstract

Anti-PD-1 therapy yields objective clinical responses in 30-40% of advanced melanoma patients. Since most patients do not respond, predictive biomarkers to guide treatment selection are needed. In view thereof, this example examines whether MHC-I/II expression is required for tumor antigen presentation and may predict response to anti-PD-1 therapy. Across 60 melanoma cell lines, bimodal expression patterns of MHC-II were found, while MHC-I expression was ubiquitous. A unique subset of melanomas are capable of expressing MHC-II under basal or IFNγ stimulated conditions. Using pathway analysis, it was found that MHC-II(+) cell lines demonstrate signatures of ‘PD-1 signaling’, ‘allograft rejection’, and ‘T-cell receptor signaling’, among others. In two independent cohorts of anti-PD-1-treated melanoma patients, MHC-II positivity on tumor cells was strongly associated with response to therapy, progression-free survival, and overall survival. MHC-II positivity also correlated with CD4+ and CD8+ tumor infiltrate. Accordingly, it was concluded that some melanomas demonstrate an autonomous MHC-II signature that correlates with anti-PD-1 response and enhanced T-cell infiltrate. MHC-II+ tumors can be robustly identified by routine melanoma-specific immunohistochemistry using commercially available antibodies for HLA-DR to improve anti-PD-1 patient selection.

Introduction

Monoclonal antibodies blocking the programmed death-1 (PD-1) receptor or its ligand (PD-L1) relieve the suppression of anti-tumor immune responses in a variety of cancers. Durable remissions occur in sizable fractions of patients with melanoma (30-40%), non-small cell lung cancer (15-20%), renal cell carcinoma (20-30%), bladder urothelial carcinoma (30%), Hodgkin lymphoma (80-90%), and others including head and neck squamous cell carcinoma and triple negative breast cancer. Accurate predictive markers of therapeutic efficacy are needed to optimize patient selection, improve treatment decision-making, and minimize costs. To date, several candidate approaches have been identified in melanoma. These include tumor or immune cell expression of PD-L1, identification of neo-antigens through next generation sequencing techniques, and T-cell receptor clonality profiling. While quite promising, these assays are technically challenging and require specialized tissue processing.

Tumors evade immune surveillance by immune checkpoint expression (PD-L1 and others), immunosuppressive cytokine profiles, tolerogenic immune cell recruitment (regulatory T-cells and others), and cancer-specific cell signaling. In addition, cancer cells can lose the ability to present tumor antigens, thus avoiding recognition by cytotoxic T cells and antigen presenting cells. Down-regulation of major histocompatibility class I and II (MHC-I and MHC-II) has been linked to immune suppression, metastatic progression, and a poor prognosis in numerous malignancies.

Despite the established importance of tumor-specific antigen expression, the influence of MHC-I and MHC-II expression on response to new immune therapies, particularly anti-PD-1/PD-L1, has not been explored. Specifically, HLA-DR is frequently expressed on melanoma and has unclear functional and prognostic significance. Without wishing to be bound by theory, it is believed that MHC-I and MHC-II expression, particularly HLA-DR, are required for anti-PD-1/PD-L1 activity and serve as technically and clinically feasible predictive biomarkers for therapeutic efficacy. As shown in this example, melanoma-specific expression of HLA-DR marks tumors with unique inflammatory signals that are more responsive to PD-1 targeted therapy. Accordingly, it is believed that tumor-specific HLA-DR expression may be used as a biomarker of high likelihood of response to these agents.

Results

Antigen presenting MHC-I and MHC-II pathways in melanoma cell lines. Based on the known biological interactions of PD-1/PD-L1 signaling, antigen presentation by tumor or professional antigen-presenting cells is hypothesized to be a requirement for immune recognition of the malignant cell. MHC-I presents antigen to CD8+ cytotoxic T lymphocytes (CTL) and is ubiquitously expressed by most cells. Loss of MHC-I is typically thought to trigger natural-killer (NK) cell checkpoints, resulting in NK-mediated cytotoxicity. In contrast, MHC-II, which presents antigen to CD4+ T helper cells, is typically restricted to professional antigen-presenting cells (APCs) such as dendritic cells and B cells. HLA-DR, the primary antigen-presenting molecule of the MHC-II pathway is expressed in some cancers, particularly in response to CTL-secreted interferon-gamma (IFNγ). Some data suggest that non-immune cells, including cancer cells can function as MHC-II+ APCs. Given the heterogeneity of the tumor milieu, a question arose as to whether MHC-I and II were expressed in in vitro cell line models of melanoma (rather than in resected melanoma tumors), where the contribution of stromal and infiltrating immune cells could be excluded.

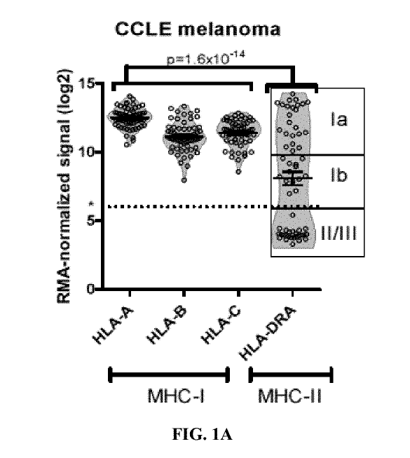

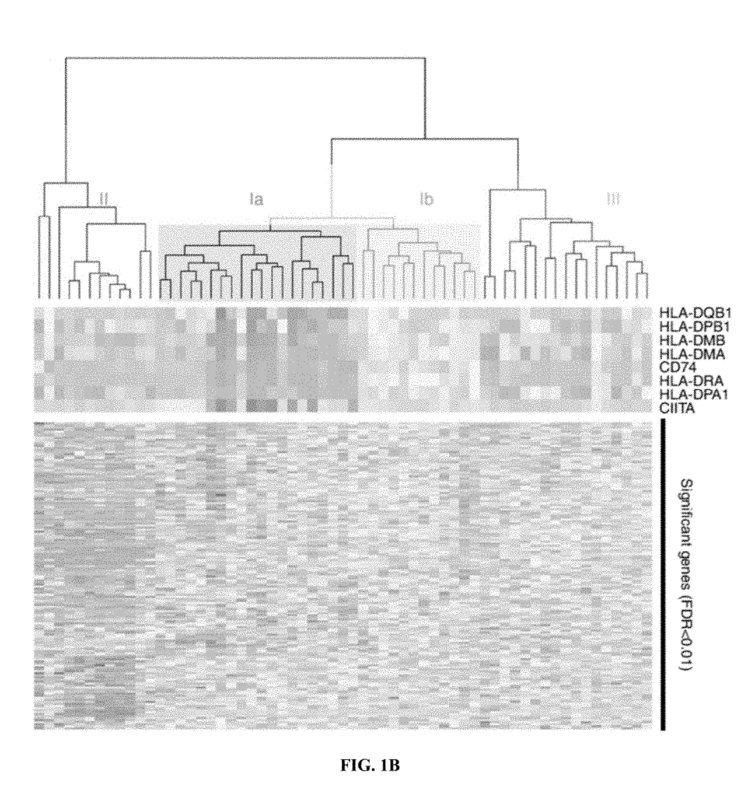

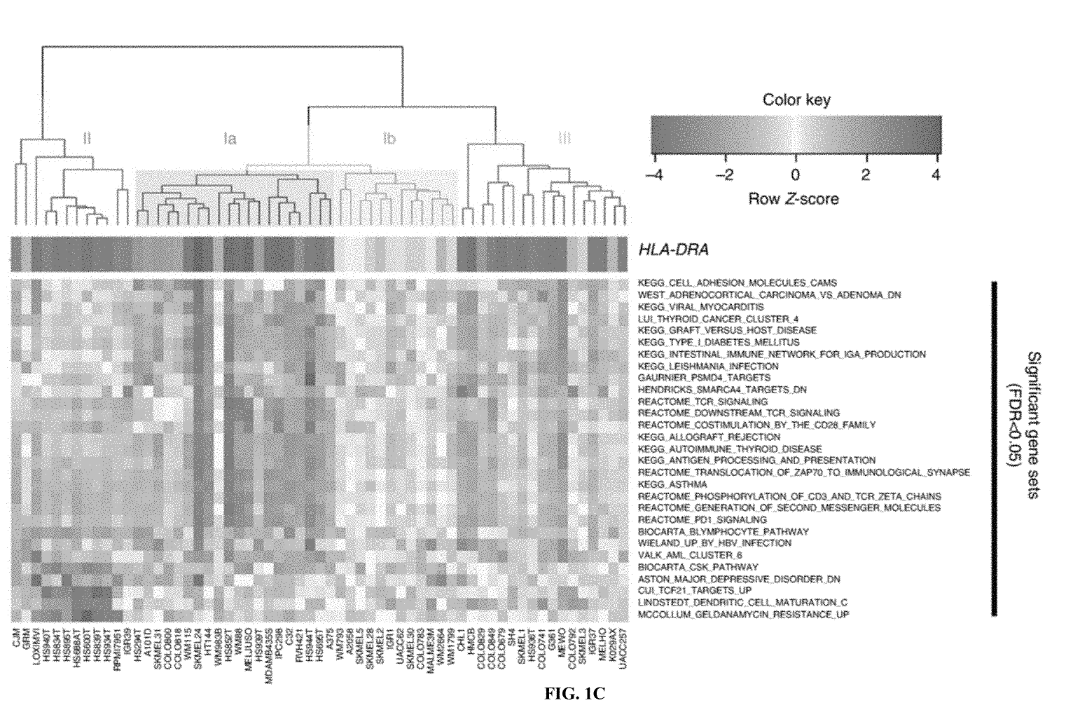

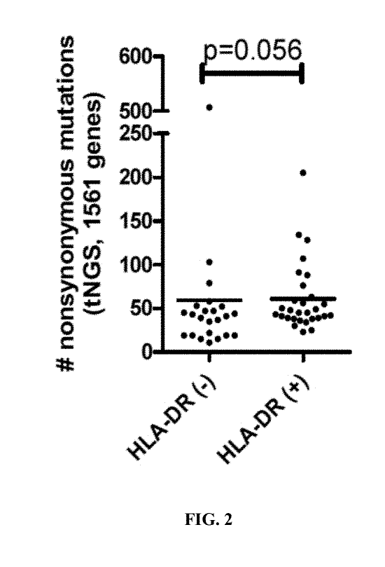

Using the Cancer Cell Line Encyclopedia (CCLE) melanoma panel of 60 cell lines, it was determined that MHC-I mRNA expression (using HLA-A as the prototype) was ubiquitously high across almost all melanoma cell lines (FIG. 1A). In contrast, HLA-DRA, the prototype MHC-II molecule, demonstrated a strong bimodal distribution pattern, and appeared absent in approximately 50% of cell lines (FIG. 1A). The remaining cell lines demonstrated intermediate to high mRNA levels. When cell lines were factored according to HLA-DRA mRNA (using an arbitrary cutoff of 6 (RMA log2 signal intensity), there was a signature of 159 genes (Table 1) which were significantly altered (up or downregulated, FDR<1%) in HLA-DRA-expressing cells compared to those cell lines lacking HLA-DRA mRNA (FIG. 1B). Clustering on these genes suggested 4 clusters of expression patterns, which were identified as clusters Ia and Ib (predominantly HLA-DR-expressing) and clusters II and III (predominantly HLA-DR-negative). Gene set analysis (GSA) of the CCLE based on MHC-II classification yielded 27 gene sets with upregulated scores and 1 with a downregulated score at an FDR≤5% in the Ia/Ib subtype. Bioinformatics analysis of the enriched gene sets suggested that HLA-DRA-expressing cell lines harbored expression signatures of ‘PD-1 signaling’, ‘T-cell receptor signaling’, ‘graft-versus-host disease’, and ‘allograft rejection’ (FIG. 1C). These findings suggested that there were tumor-cell autonomous signaling pathways driving MHC-II expression consistent with a pro-immune/anti-tumor response. The presence of a high mutational burden and resulting neoantigens has been shown to predict response to PD-L1 therapy in lung cancer. HLA-DR-expressing melanoma lines had a higher total nonsynonymous mutational load by targeted next-generation sequencing of 1,561 genes, although this was not statistically significant (Wilcoxon Rank Sum p=0.056, FIG. 2).

TABLE 1
Genes altered in MHC-II (+) melanoma cell lines versus
MHC-II (−) cell lines

| | | Log2 Fold | | |
|---|---|---|---|---|
| | | Change | | |
| | | HLA-DR+ | | False |
| | | versus | P-value | Discovery |
| Gene ID | T-statistic | HLA-DR− | (uncorrected) | Rate |
| HLA-DRA | 15.213927 | 6.8995504 | 4.38E−22 | 8.80E−18 |
| CD74 | 10.845865 | 5.4611784 | 1.12E−15 | 1.13E−11 |
| HLA-DPA1 | 9.851692 | 5.0791636 | 4.49E−14 | 3.01E−10 |
| S100B | 5.592843 | 4.2563749 | 6.06E−07 | 3.58E−04 |
| PLP1 | 4.849015 | 3.5828047 | 9.41E−06 | 2.56E−03 |
| HLA-DMA | 8.661834 | 3.5538701 | 4.20E−12 | 2.11E−08 |
| SNX10 | 5.406524 | 3.3458826 | 1.22E−06 | 6.28E−04 |
| FABP7 | 5.594157 | 3.3277273 | 6.03E−07 | 3.58E−04 |
| NFATC2 | 6.060418 | 3.3201765 | 1.03E−07 | 9.83E−05 |
| PTPRZ1 | 5.176045 | 3.0896493 | 2.86E−06 | 1.01E−03 |
| NGFR | 5.925755 | 3.0076695 | 1.72E−07 | 1.50E−04 |
| FLRT3 | 6.584844 | 2.9601229 | 1.36E−08 | 2.73E−05 |
| MMP8 | 4.697741 | 2.9277593 | 1.62E−05 | 3.70E−03 |
| TGFA | 4.705974 | 2.800589 | 1.57E−05 | 3.70E−03 |
| GAS7 | 6.389378 | 2.7747686 | 2.90E−08 | 3.64E−05 |
| ERBB3 | 4.824419 | 2.7459398 | 1.03E−05 | 2.69E−03 |
| ROPN1 | 5.313675 | 2.7099096 | 1.72E−06 | 7.56E−04 |
| MIA | 5.198919 | 2.6799177 | 2.63E−06 | 9.79E−04 |
| HLA-DPB1 | 5.293034 | 2.6171656 | 1.86E−06 | 7.94E−04 |
| ASB9 | 6.514841 | 2.5990046 | 1.78E−08 | 2.99E−05 |
| SHROOM2 | 6.779445 | 2.592674 | 6.38E−09 | 1.83E−05 |
| TRIM9 | 6.482923 | 2.5090333 | 2.02E−08 | 3.05E−05 |
| LYPD1 | 6.106807 | 2.4834576 | 8.59E−08 | 8.64E−05 |
| SYNM | 6.469599 | 2.4805447 | 2.12E−08 | 3.05E−05 |
| EPHA3 | 4.355389 | 2.4732033 | 5.37E−05 | 7.61E−03 |
| SLC35F1 | 6.166944 | 2.4706035 | 6.82E−08 | 7.22E−05 |
| HLA-DQB1 | 4.789133 | 2.4531794 | 1.17E−05 | 2.97E−03 |
| SORBS2 | 4.834557 | 2.4447783 | 9.91E−06 | 2.66E−03 |
| GNG2 | 6.685527 | 2.4378914 | 9.20E−09 | 2.31E−05 |
| UGT8 | 4.829512 | 2.3379229 | 1.01E−05 | 2.67E−03 |
| ITGB3 | 5.082412 | 2.2978034 | 4.03E−06 | 1.29E−03 |
| SLITRK6 | 4.386808 | 2.2690057 | 4.82E−05 | 7.02E−03 |
| ITGB8 | 5.056133 | 2.2372412 | 4.44E−06 | 1.39E−03 |
| TRPM8 | 4.466726 | 2.2089335 | 3.65E−05 | 5.77E−03 |
| HLA-DMB | 7.729596 | 2.2029647 | 1.57E−10 | 6.31E−07 |
| TMTC2 | 6.011463 | 2.1882044 | 1.24E−07 | 1.13E−04 |
| SHC4 | 5.106057 | 2.1758035 | 3.70E−06 | 1.21E−03 |
| POU3F2 | 5.709246 | 2.0811539 | 3.91E−07 | 2.64E−04 |
| TFAP2C | 4.29478 | 2.0657408 | 6.62E−05 | 8.58E−03 |
| SYTL2 | 5.319357 | 2.0496798 | 1.68E−06 | 7.56E−04 |
| TNFRSF21 | 5.547299 | 2.0424787 | 7.19E−07 | 4.02E−04 |
| AGPAT9 | 5.262261 | 2.0308708 | 2.08E−06 | 8.60E−04 |
| FAM78B | 4.641567 | 2.0234908 | 1.97E−05 | 3.96E−03 |
| ST6GAL1 | 4.444329 | 2.0159904 | 3.95E−05 | 5.92E−03 |
| TM4SF18 | 4.526559 | 2.0130853 | 2.96E−05 | 5.19E−03 |
| HIVEP3 | 5.186298 | 1.9981964 | 2.75E−06 | 9.89E−04 |
| CLMN | 6.559112 | 1.9552922 | 1.50E−08 | 2.75E−05 |
| TIAM1 | 5.666435 | 1.9343871 | 4.59E−07 | 2.89E−04 |
| SIPA1L2 | 5.103054 | 1.9284935 | 3.74E−06 | 1.21E−03 |
| FHDC1 | 7.649529 | 1.9267439 | 2.15E−10 | 7.19E−07 |
| FREM2 | 4.675119 | 1.8894848 | 1.75E−05 | 3.74E−03 |
| SLC26A2 | 6.429413 | 1.8663853 | 2.48E−08 | 3.33E−05 |
| RASSF4 | 4.881541 | 1.8639941 | 8.37E−06 | 2.35E−03 |
| SPATA13 | 5.896206 | 1.8077273 | 1.92E−07 | 1.61E−04 |
| FRMD5 | 5.455314 | 1.7843982 | 1.02E−06 | 5.37E−04 |
| RNF125 | 4.273905 | 1.7842557 | 7.11E−05 | 9.06E−03 |
| B4GALT6 | 5.73706 | 1.7646051 | 3.52E−07 | 2.53E−04 |
| ATP10B | 4.362509 | 1.7340418 | 5.24E−05 | 7.51E−03 |
| TMEM171 | 4.636609 | 1.7333361 | 2.01E−05 | 3.96E−03 |
| RTP4 | 4.697952 | 1.6884598 | 1.62E−05 | 3.70E−03 |
| KAT2B | 5.378765 | 1.685032 | 1.35E−06 | 6.68E−04 |
| FOXD3 | 4.424441 | 1.6731433 | 4.23E−05 | 6.21E−03 |
| CSRP2 | 4.315628 | 1.6639551 | 6.16E−05 | 8.21E−03 |
| CHST6 | 5.137389 | 1.6217742 | 3.30E−06 | 1.12E−03 |
| LPCAT2 | 5.707336 | 1.5952037 | 3.93E−07 | 2.64E−04 |
| PREX1 | 4.511328 | 1.5765817 | 3.12E−05 | 5.24E−03 |
| TNC | 4.670963 | 1.5484352 | 1.78E−05 | 3.74E−03 |
| CIITA | 5.169709 | 1.5433302 | 2.93E−06 | 1.02E−03 |
| MARCKSL1 | 5.376473 | 1.5427519 | 1.36E−06 | 6.68E−04 |
| INPP5F | 4.62986 | 1.5315523 | 2.06E−05 | 4.02E−03 |
| SAMD5 | 4.553002 | 1.4635345 | 2.70E−05 | 4.89E−03 |
| PLXNB3 | 5.209431 | 1.4616735 | 2.53E−06 | 9.79E−04 |
| MAML3 | 4.461074 | 1.459821 | 3.72E−05 | 5.79E−03 |
| GLDC | 4.334158 | 1.4477283 | 5.78E−05 | 7.91E−03 |
| ST3GAL4 | 4.66421 | 1.433675 | 1.82E−05 | 3.76E−03 |
| KHDRBS3 | 4.722392 | 1.427277 | 1.48E−05 | 3.59E−03 |
| LDLRAD4 | 4.561478 | 1.427108 | 2.62E−05 | 4.79E−03 |
| ITGA6 | 5.199928 | 1.4205761 | 2.62E−06 | 9.79E−04 |
| PMP22 | 5.321876 | 1.4010431 | 1.67E−06 | 7.56E−04 |
| BFSP1 | 5.048322 | 1.3644542 | 4.57E−06 | 1.39E−03 |
| EXTL1 | 5.504541 | 1.3540492 | 8.44E−07 | 4.59E−04 |
| GULP1 | 4.361196 | 1.3323331 | 5.26E−05 | 7.51E−03 |
| ACP6 | 5.048185 | 1.3318244 | 4.57E−06 | 1.39E−03 |
| TNS3 | 5.235553 | 1.3236621 | 2.30E−06 | 9.24E−04 |
| SDC3 | 4.734534 | 1.3226811 | 1.42E−05 | 3.48E−03 |
| ZNF827 | 4.780905 | 1.2975313 | 1.20E−05 | 2.98E−03 |
| SPATA6 | 5.187201 | 1.2373858 | 2.75E−06 | 9.89E−04 |
| PAQR8 | 4.625539 | 1.1707259 | 2.09E−05 | 4.04E−03 |
| CUBN | 4.525968 | 1.1344845 | 2.97E−05 | 5.19E−03 |
| DAG1 | 6.205058 | 1.1210876 | 5.89E−08 | 6.58E−05 |
| ABCB9 | 6.603694 | 1.1156536 | 1.26E−08 | 2.73E−05 |
| SNRPB2 | 4.672014 | 1.0888635 | 1.77E−05 | 3.74E−03 |
| LINC00327 | 4.956843 | 1.0438048 | 6.37E−06 | 1.88E−03 |
| PYGB | 4.662699 | 1.0210988 | 1.83E−05 | 3.76E−03 |
| HOXC13 | 4.304304 | 1.0195866 | 6.40E−05 | 8.47E−03 |
| TMX4 | 4.322439 | 0.9882031 | 6.02E−05 | 8.12E−03 |
| DPY19L1 | 4.454231 | 0.9013517 | 3.81E−05 | 5.85E−03 |
| SPRY2 | 4.6715 | 0.8697505 | 1.78E−05 | 3.74E−03 |
| HPS5 | 5.203752 | 0.8356998 | 2.58E−06 | 9.79E−04 |
| SRGAP2 | 4.924721 | 0.8285677 | 7.16E−06 | 2.06E−03 |
| CDK5 | 4.58497 | 0.8165132 | 2.41E−05 | 4.52E−03 |
| IFNGR2 | 5.825123 | 0.7982793 | 2.52E−07 | 1.95E−04 |
| DCPS | 4.378902 | 0.7639304 | 4.95E−05 | 7.16E−03 |
| SHROOM4 | 4.94164 | 0.7509587 | 6.73E−06 | 1.96E−03 |
| PIAS2 | 4.348352 | 0.7476783 | 5.50E−05 | 7.74E−03 |
| ITPK1 | 4.636603 | 0.7457709 | 2.01E−05 | 3.96E−03 |
| CD58 | 4.517366 | 0.7443327 | 3.06E−05 | 5.24E−03 |
| ADCK3 | 4.315287 | 0.7371069 | 6.17E−05 | 8.21E−03 |
| VPS37B | 5.796215 | 0.7347872 | 2.81E−07 | 2.09E−04 |
| ABTB2 | 4.451921 | 0.7210315 | 3.84E−05 | 5.86E−03 |
| RFX5 | 4.47776 | 0.6708229 | 3.51E−05 | 5.69E−03 |
| PITPNM2 | 4.694316 | 0.6704875 | 1.64E−05 | 3.70E−03 |
| CTSE | 4.543744 | 0.6652288 | 2.79E−05 | 4.99E−03 |
| CCSAP | 4.4481 | 0.6474003 | 3.90E−05 | 5.89E−03 |
| NFKB1 | 4.999332 | 0.635211 | 5.46E−06 | 1.64E−03 |
| ABCF2 | 4.617906 | 0.622915 | 2.15E−05 | 4.11E−03 |
| ZCCHC17 | 4.701173 | 0.5407589 | 1.60E−05 | 3.70E−03 |
| GUCD1 | 4.338599 | 0.5142809 | 5.69E−05 | 7.89E−03 |
| NUBP1 | 4.325669 | 0.4947412 | 5.95E−05 | 8.08E−03 |
| ZC3H4 | 4.612174 | 0.4700155 | 2.19E−05 | 4.16E−03 |
| ZMIZ2 | 4.563336 | 0.4554563 | 2.60E−05 | 4.79E−03 |
| LOC729870 | 4.466489 | 0.4304289 | 3.65E−05 | 5.77E−03 |
| BTBD16 | 4.298153 | 0.1890444 | 6.54E−05 | 8.54E−03 |
| MSR1 | −4.426413 | −0.3551014 | 4.20E−05 | 6.21E−03 |
| LOC100130417 | −4.707529 | −0.3555586 | 1.56E−05 | 3.70E−03 |
| SMARCAL1 | −4.334969 | −0.381422 | 5.76E−05 | 7.91E−03 |
| KIAA1407 | −4.273546 | −0.4475706 | 7.12E−05 | 9.06E−03 |
| LMBR1L | −4.465177 | −0.4563252 | 3.67E−05 | 5.77E−03 |
| RAB10 | −4.682295 | −0.4721377 | 1.71E−05 | 3.74E−03 |
| INPP5E | −4.342394 | −0.4818347 | 5.62E−05 | 7.84E−03 |
| TANGO6 | −4.287174 | −0.5897189 | 6.79E−05 | 8.75E−03 |
| LCA5 | −4.670828 | −0.6010548 | 1.78E−05 | 3.74E−03 |
| LINC00959 | −4.459301 | −0.6769525 | 3.75E−05 | 5.79E−03 |
| DDHD1 | −5.56089 | −0.7373122 | 6.83E−07 | 3.93E−04 |
| SEMA3F | −5.312191 | −0.8999891 | 1.73E−06 | 7.56E−04 |
| PTGR1 | −5.837171 | −0.9936534 | 2.41E−07 | 1.93E−04 |
| ANKRD33B | −4.583103 | −1.0284012 | 2.43E−05 | 4.52E−03 |
| TRIM61 | −4.522971 | −1.0557596 | 3.00E−05 | 5.20E−03 |
| PSTPIP2 | −4.512212 | −1.0708537 | 3.11E−05 | 5.24E−03 |
| HEBP2 | −4.855401 | −1.1343351 | 9.20E−06 | 2.53E−03 |
| NFIL3 | −5.131065 | −1.1961312 | 3.37E−06 | 1.13E−03 |
| METRNL | −5.367948 | −1.2970141 | 1.41E−06 | 6.73E−04 |
| ZNF585B | −4.475736 | −1.2989619 | 3.54E−05 | 5.69E−03 |
| SERINC2 | −4.669901 | −1.331719 | 1.79E−05 | 3.74E−03 |
| SH2D4A | −4.260014 | −1.3650528 | 7.45E−05 | 9.43E−03 |
| TBC1D2 | −4.483972 | −1.3749573 | 3.44E−05 | 5.66E−03 |
| STEAP2 | −4.300687 | −1.3925252 | 6.48E−05 | 8.52E−03 |
| PTPRB | −4.879853 | −1.4727397 | 8.42E−06 | 2.35E−03 |
| NOTCH3 | −4.51109 | −1.5696335 | 3.13E−05 | 5.24E−03 |
| DUSP1 | −4.785775 | −1.6339789 | 1.18E−05 | 2.97E−03 |
| PRKG1 | −5.26052 | −1.6924671 | 2.09E−06 | 8.60E−04 |
| MAN1A1 | −4.541875 | −1.7979765 | 2.81E−05 | 4.99E−03 |
| LOXL1-AS1 | −5.684096 | −1.8588609 | 4.30E−07 | 2.79E−04 |
| PLAGL1 | −4.482 | −2.4807309 | 3.46E−05 | 5.66E−03 |
| FHL1 | −4.486256 | −2.5086713 | 3.41E−05 | 5.66E−03 |
| FOXP1 | −6.314977 | −2.677233 | 3.86E−08 | 4.56E−05 |
| DSP | −4.654542 | −2.7856211 | 1.89E−05 | 3.83E−03 |
| LOXL1 | −4.807904 | −2.9499098 | 1.09E−05 | 2.81E−03 |
| CDH11 | −4.430404 | −3.3173098 | 4.14E−05 | 6.17E−03 |

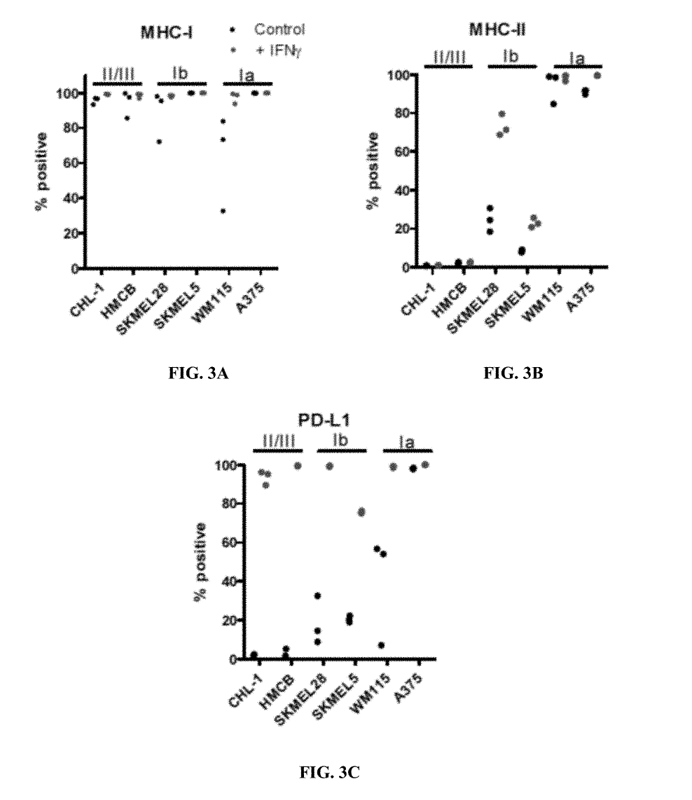

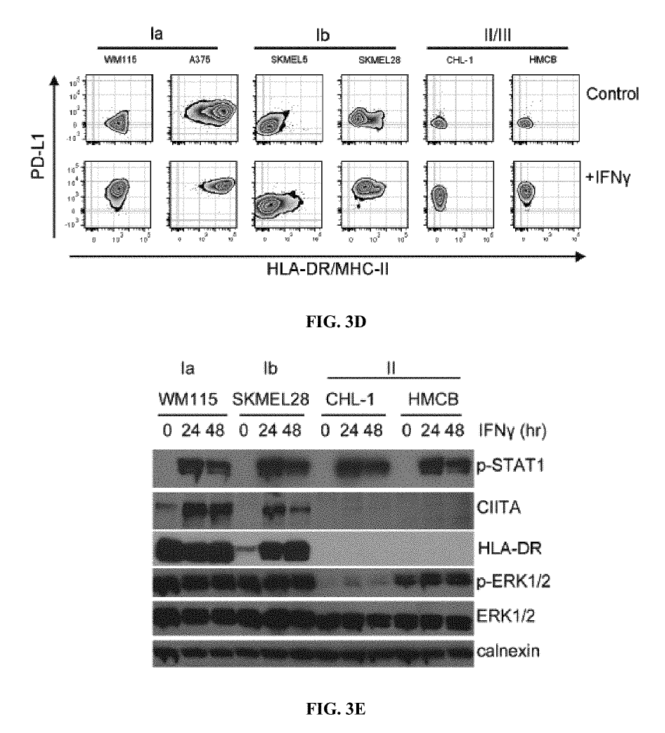

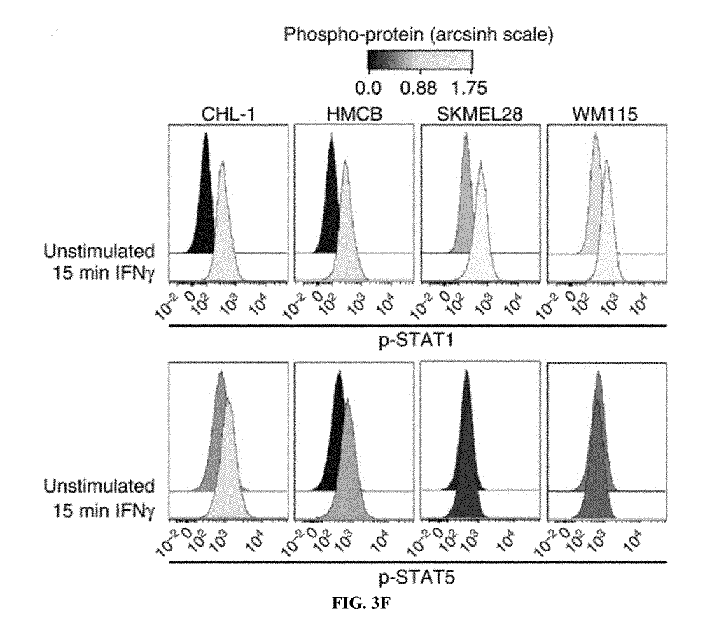

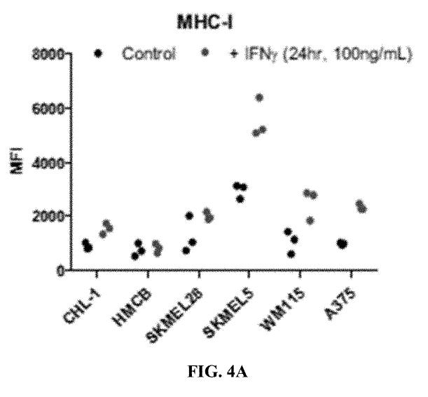

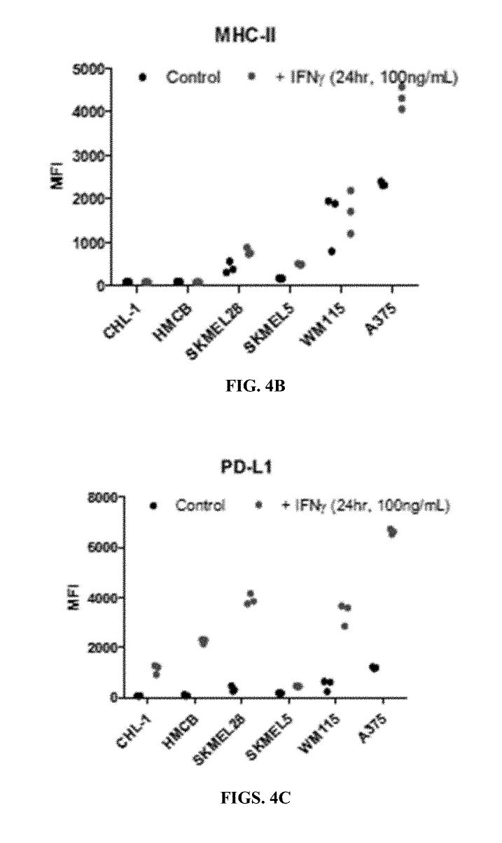

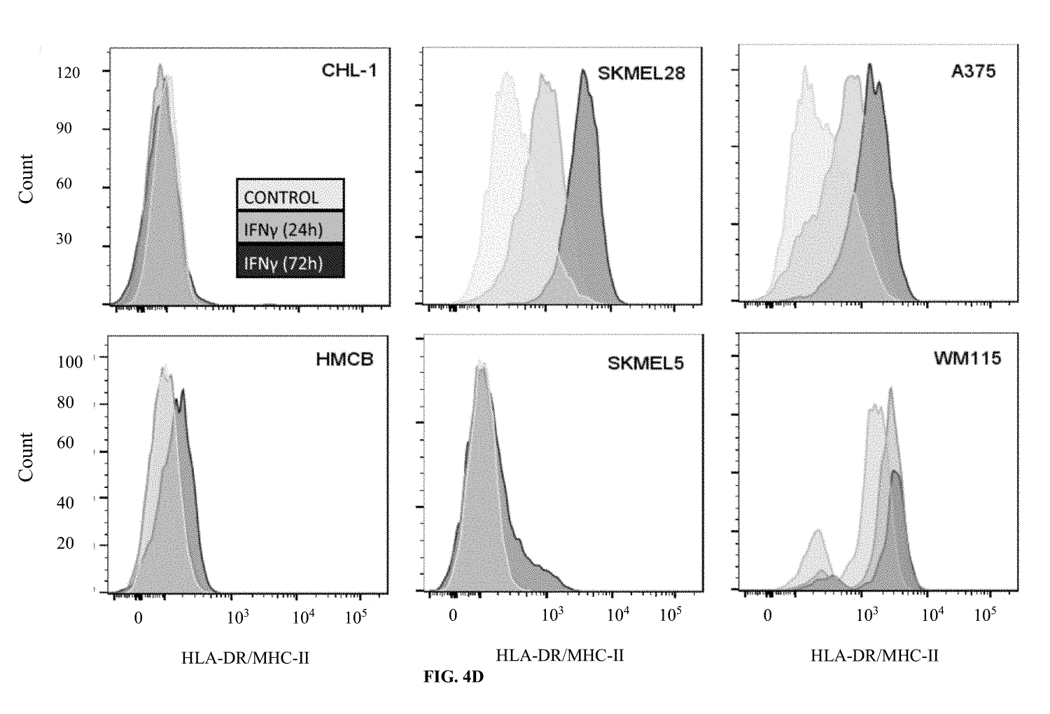

Since mRNA expression does not imply functional protein expression, and because micro-environmental IFNγ is known to influence MHC-I, MHC-II and PD-L1 expression, representative cell lines from HLA-DRA-expressing (cluster Ia and Ib, FIG. 1B) and HLA-DRA-deficient (cluster II, FIG. 1B) subgroups were characterized by flow cytometry under basal and stimulated (IFNγ) conditions. Cell surface expression mirrored mRNA expression patterns; MHC-I (HLA-A/B/C) was expressed ubiquitously among all cell lines under both basal and stimulated conditions, while MHC-II (HLA-DR) was present only on the intermediate/Ib (SKMEL5 and SKMEL28) and high/Ia cell lines (WM115 and A375; FIGS. 3A-C and 4A-D). No significant increase in HLA-DR expression was observed with either CHL-1 or HMCB even after 72 hrs of IFNγ treatment (FIG. 4D) Notably, the intermediate/Ib cell line SKMEL28 had a unique population (25%) of cells that was constitutively HLA-DR-expressing at baseline, and was potently induced by IFNγ (FIG. 3D). The high (Ia) WM115 cell line was essentially 100% positive for HLA-DR at both basal and stimulated conditions.

Interestingly, PD-L1 expression was potently induced with stimulation in all cell lines, though the HLA-DR+ cell lines exhibited greater populations of cells that were PD-L1 positive in the absence of IFNγ (FIGS. 3C-D). Consistent with this, STAT1 was robustly activated with IFNγ stimulation in all cell lines whereas CIITA expression, a master regulator of MHC-II transcription, was only induced in HLA-DR+ Ia/Ib cells (FIG. 3E). Phospho-flow analysis demonstrated that while STAT1 was activated robustly with short-term (15 min) IFNγ stimulation, STATS was preferentially activated by IFNγ in MHC-II(−) cell lines (FIG. 3F), consistent with the observations of others that STATS can contribute to resistance to interferon signaling and phenotypes. Together, these results suggest that there is a tumor-cell autonomous inflammatory signal present in a subset of melanomas that may predispose the tumor to enhanced MHC-II expression, antigen presentation (direct or cross-presentation via exosomes) to CD4+ T helper cells, and immune recognition, coinciding with higher PD-L1 expression. Furthermore, these data suggest that STATS activation may contribute to suppression of this inflammatory signal. Thus, it was reasoned that the HLA-DR-expressing subtype of melanoma can be unmasked to the immune system by therapeutic inhibition of the PD-1/PD-L1 axis.

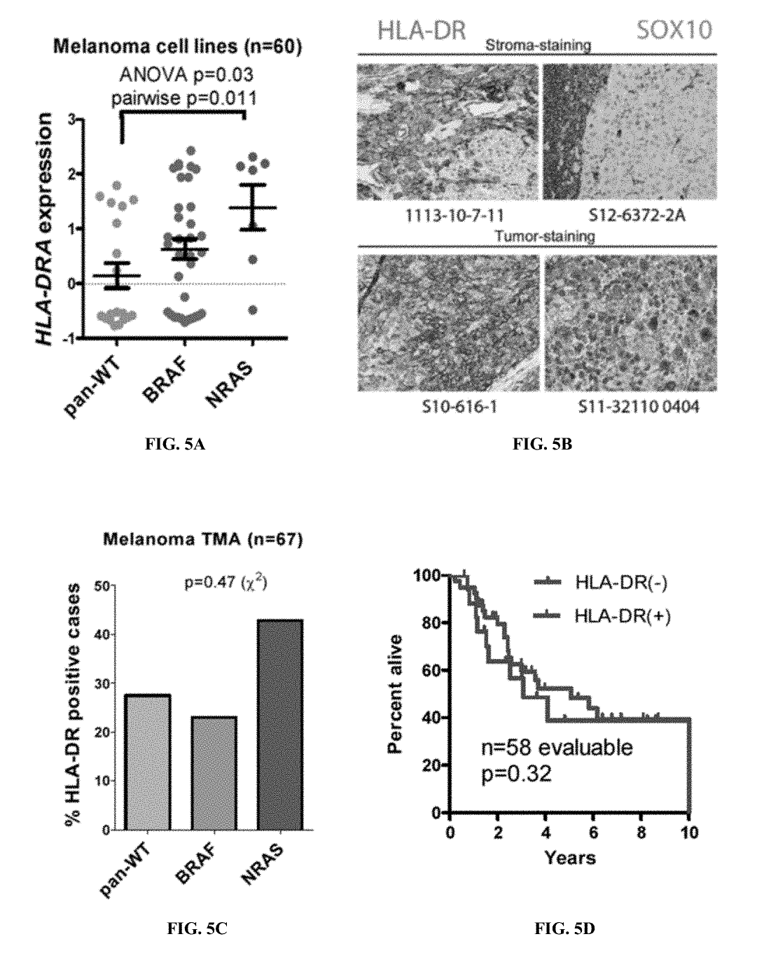

HLA-DR expression by genotype. HLA-DRA expression was specifically enriched in cell lines harboring NRAS mutations (FIG. 5A). Notably, studies suggested that patients harboring NRAS mutations experience improved response rates to PD-1 axis therapy and other immune therapies. To confirm this clinically, MHC-II/HLA-DR expression by IHC was first investigated in a tissue microarray (TMA) of melanoma patient samples (n=67) with known BRAF and NRAS genotypes who largely had not received immune therapy (Table 2). Dual-color IHC was performed with HLA-DR and SOX10 to distinguish tumor vs. stromal expression of HLA-DR (FIG. 5B). HLA-DR (+) tumor expression was observed in 20/67 (30%) evaluable samples. Similar to cell line RNA analysis, HLA-DR was expressed more frequently in the NRAS-mutated cohort (43%, 6 of 14) than in BRAF-mutated (23%, 3 of 13) and BRAF/NRAS wild type populations (28%, 11 of 39) (FIG. 5C), but this was not statistically significant χ² p=0.47). Thus, NRAS genotype seems to trend with HLA-DR positivity, but this association does not appear to be a significant in patients. A larger analysis would be needed to determine whether this association is apparent in patients. Importantly, in this unselected population of patients, expression of HLA-DR was not associated with overall survival (p=0.32), suggesting that HLA-DR expression may not be generally prognostic in advanced melanoma (FIG. 5D).

TABLE 2
Association of HLA-DR staining on melanoma tissue microarray
with clinical variables (N = 66)

| | HLA-DR (+) | HLA-DR (−) | |
|---|---|---|---|
| | N = 20 | N = 46 | P value |
| Age (average, years) | 57.1 | 61.0 | 0.323 |
| Gender | | | |
| Male | 12 (60%) | 31 (67%) | 0.562 |
| Female | 8 (40%) | 15 (33%) | |
| Stage at resection/biopsy | | | |
| I-II | 2 (10%) | 6 (13%) | 0.755 |
| III | 6 (30%) | 17 (37%) | |
| IV | 12 (60%) | 23 (50%) | |
| LDH Elevated | 2 (10%) | 10 (22%) | 0.149 |
| Mutation | | | |
| BRAF^{V600} | 3 (15%) | 10 (22%) | 0.485 |
| NRAS^{Q51/G12/G13} | 6 (30%) | 8 (17%) | |
| BRAF/NRAS wild type | 11 (55%) | 28 (61%) | |
| Primary tumor ulceration | 7 (35%) | 15 (33%) | 0.124* |
| Metastatic disease | 18 (90%) | 35 (76%) | 0.192 |
| Liver involvement^{#} | 2 (11%) | 14 (40%) | 0.030 |
| Lung involvement^{#} | 10 (56%) | 24 (69%) | 0.349 |
| Brain involvement^{#} | 7 (39%) | 8 (35%) | 0.220 |
| Median survival | 35.0 mo | 35.0 mo | 0.950 |
| 95% confidence interval | 4.3-65.7 mo | 0-78.2 mo | |

*Ulceration status unknown in 20 patients # Expressed as percentage of patients with metastatic disease

HLA-DR expression in patients receiving anti-PD-1/PD-L 1. The instant inventors previously observed that in a diverse collection of melanoma cell lines, patterns of HLA-DR expression were 1) constitutively high, 2) heterogeneous, but inducible by IFNγ, or 3) constitutively off. Similar patterns were observed in a cohort of unselected melanoma tumors, and thus it was hypothesized that these patterns may be predictive of benefit to immunotherapy.

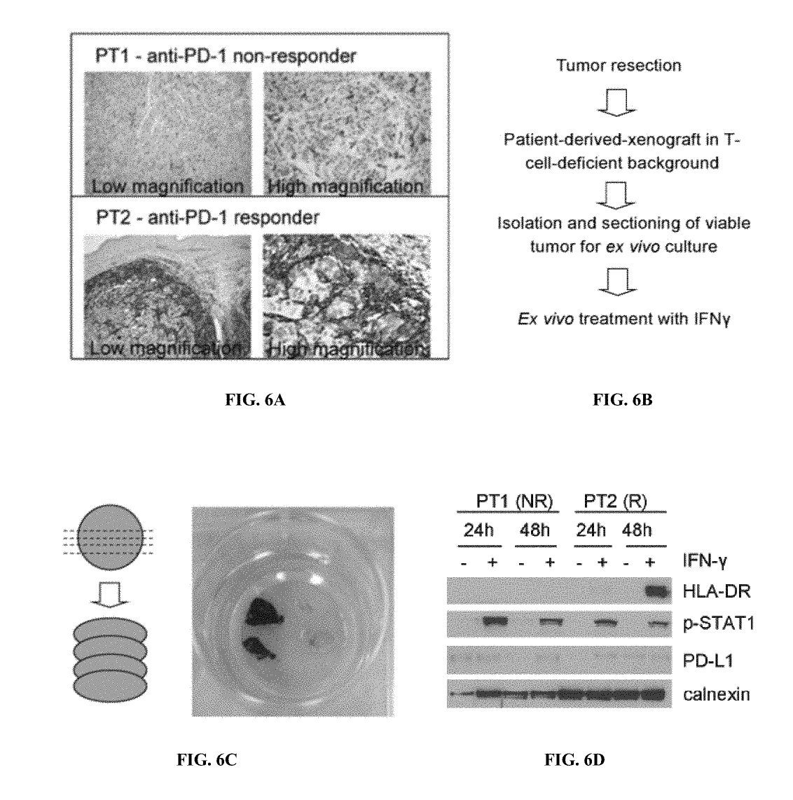

To test this hypothesis, patient-derived xenograft (PDX) models were utilized from the tumor resections of two melanoma patients who subsequently received anti-PD1 therapy; patient 1 (PT1; non-responder, 0% HLA-DR-positive, class II/III) and patient 2 (PT2; partial responder, heterogeneous 15% HLA-DR-positive, class Ib) (FIG. 6A). In PT2, the HLA-DR staining pattern was clearly positive at the invasive interface, suggesting immune-reactivity in this particular tumor, in contrast to other tumors identified in the TMA study which were MHC-II(+) throughout the tumor. The resected tumors from PT1 and PT2 were serially transplanted to athymic nu/nu mice, which are highly deficient in functional T cells, ruling out a possible source of IFNγ (FIG. 6B). Immunohistochemistry analysis of both PDX models, grown in nude mice, demonstrated no detectable HLA-DR expression (data not shown). However, when PDX tumors were freshly resected, sectioned and grown ex vivo as cultured tissue slices, in the presence or absence of IFNγ, only the PT2 PDX model (anti-PD-1 responder) upregulated HLA-DR (FIG. 6D). Thus, HLA-DR may be a marker of IFNγ activity in the microenvironment of some (but not all) tumors. Furthermore, this experiment supports the notion that the IFNγ response varies significantly among melanomas, and demonstrates tumor-autonomous features. Furthermore, these data suggest that HLA-DR expression in melanoma cells may be a biomarker for tumors primed with activated T-cells and an appropriate IFNγ response to mediate sensitivity to PD-1/PD-L1 blockade. Importantly, however, these data do not rule out the existence of melanomas constitutively-expressing HLA-DR in the absence of IFNγ stimulation, as is observed in a significant number of melanoma cell line models (FIG. 1).

In order to determine whether MHC-II expression on melanoma tumors is associated with clinical response to PD-1/PD-L1 targeted therapy, archival pre-treatment biopsy or resection specimens were obtained from 30 patients treated with anti-PD-1 (nivolumab, pembrolizumab) or anti-PD-L1 (MPDL3280A; n=2). The median age was 56 years, the median number of prior therapies was 1, and 14 (47%) had failed ipilimumab (Table 3). Twenty-three patients (77%) had stage IV M1c disease and 12 (40%) had elevated serum lactate dehydrogenase (LDH).

TABLE 3
Clinical characteristics of patients treated with anti-PD-
1/PD-L1 (Discovery cohort, n = 30)

| | Number | Percentage |
|---|---|---|
| Age | 56 (median) | 27-81 (range) |
| Gender | | |
| Male | 16 | 53 |
| Female | 14 | 47 |
| Stage | | |
| M1a | 3 | 10 |
| M1b | 4 | 13 |
| M1c | 23 | 77 |
| LDH Elevated | 12 | 40 |
| Mutation | | |
| BRAF V600 | 6 | 20 |
| NRAS Q61 | 7 | 23 |
| BRAF/NRAS wild type | 17* | 57 |
| Prior therapies | 1 (median) | 0-3 (range) |
| IL-2 | 5 | 20 |
| Ipilimumab | 14 | 47 |
| BRAF +/− MEK inhibitor | 4 | 13 |
| Cytotoxic chemotherapy | 5 | 17 |

*NRAS status unknown on 2 patients

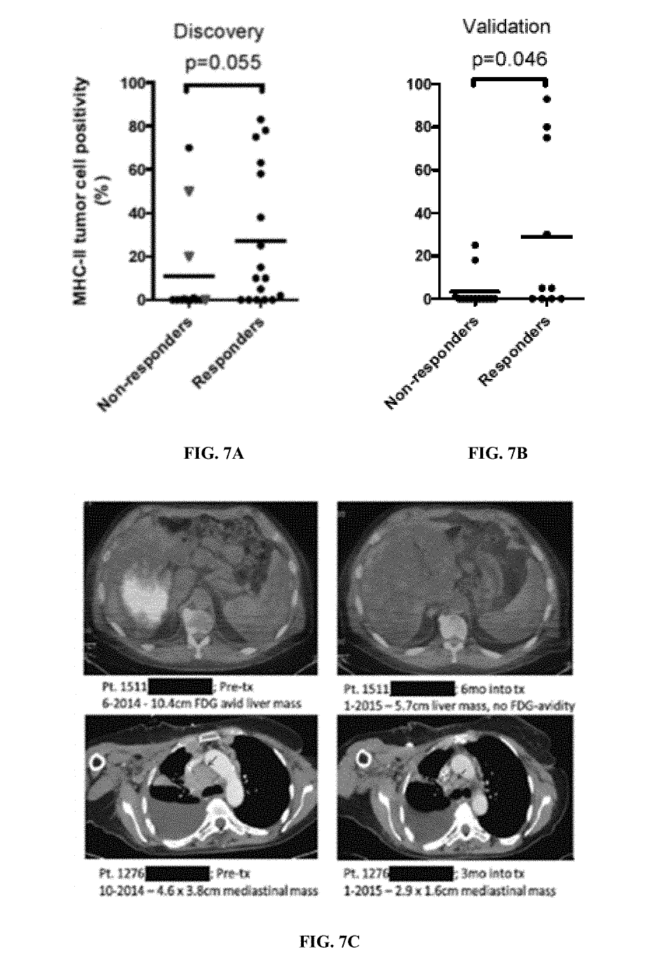

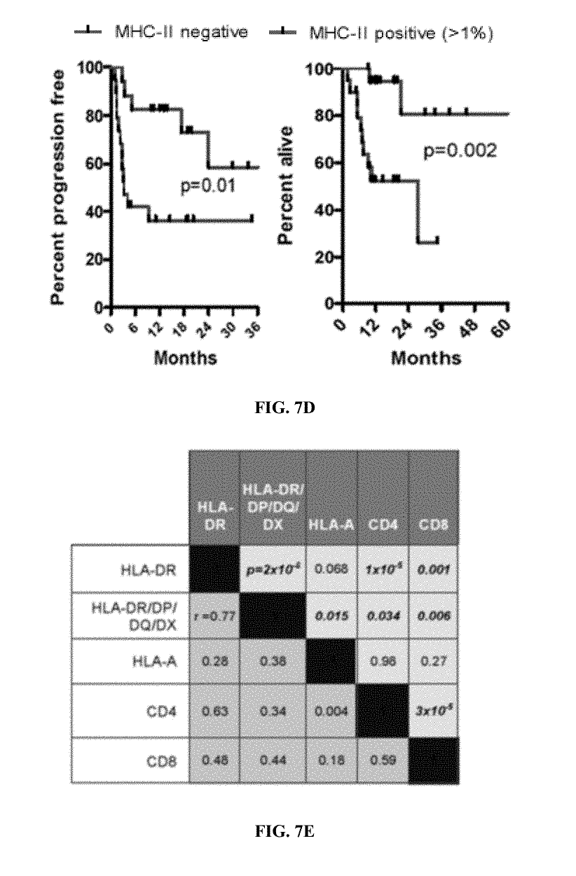

MHC-II+ from MHC-II- samples were differentiated using a cutoff of >1% of tumor (SOX10+) membranes showing staining. However, the vast majority of positive samples were positive in greater than 5% of tumor cells in the entire section; only one positive sample within the cohort scored at the 2% range. HLA-DR staining strongly correlated with response to therapy. Among 14 patients with positive HLA-DR staining (>1% estimation of positive tumor membranes in the entire tissue section), 11 patients (79%) had complete (n=3) or partial (n=8) response (FIG. 7A). Clinical activity was inferior in HLA-DR non-expressing melanomas; 6 of 16 patients (38%) responded to therapy (ORR 79% vs. 38%, Fisher's Exact p=0.033). Clinical benefit (including mixed responses) was similarly superior in MHC-II(+) patients (Fisher's Exact p=0.007). Importantly, this finding was confirmed in a second independent dataset of 23 melanoma patients treated with anti-PD-1 therapy (single agent or concurrently with other immunotherapies). Of these 23 additional patients, 6/8 (75%) of HLA-DR(+) tumors responded (PR or CR), while only 4/15 (27%) HLA-DR(−) responded (Fisher's Exact p=0.025) (FIG. 7B). Rapid objective clinical responses were observed in HLA-DR(+) tumors, even in patients with other negative prognostic features, including a patient with bulky disease, elevated LDH, impaired functional status, and failure of both ipilimumab and dabrafenib/trametinib, and a patient with a >10 cm liver mass and LDH >500 unit/L following failure of interleukin-2 and ipilimumab (FIG. 7C).

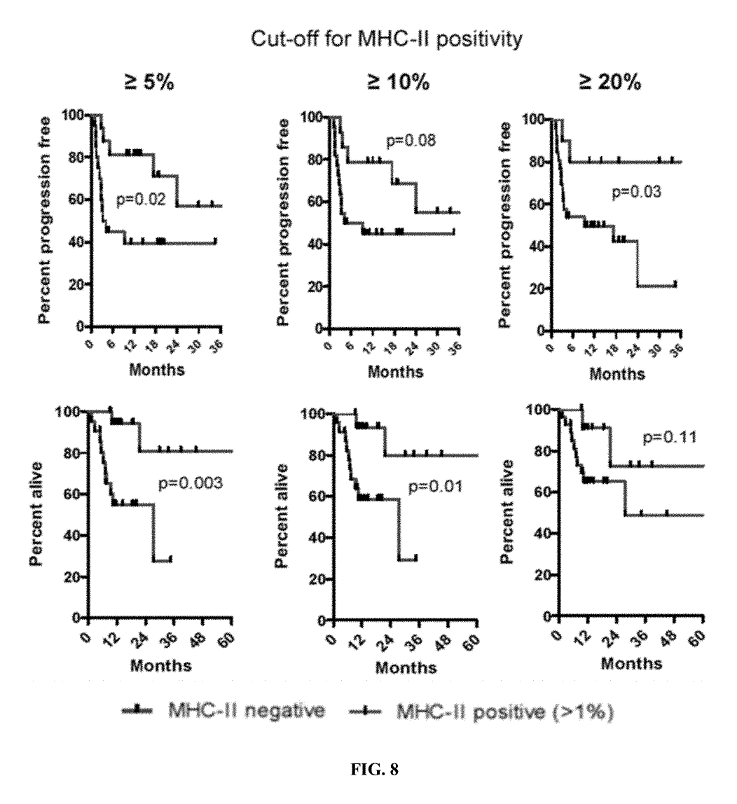

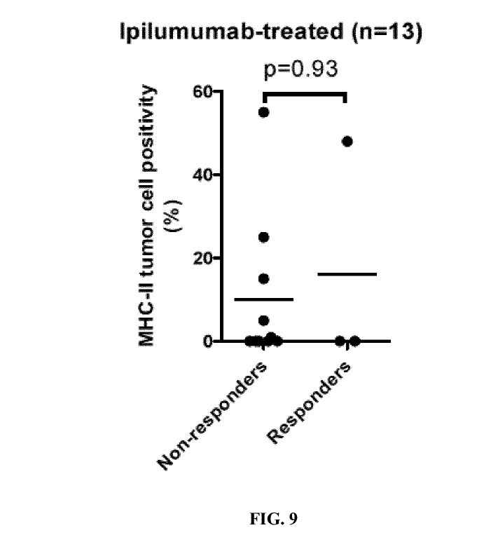

Progression-free survival (PFS) between patient groups in both datasets was also compared, when survival data were available. The median PFS was superior in the HLA-DR (+) group (median not reached vs. 3.2 months, log-rank p=0.01; FIG. 7D). Overall survival was also superior for the HLA-DR (+) cohort (median not reached vs. 27.5 months, log-rank p=0.002; FIG. 7D). The 3 patients with mixed responses from the PFS analysis (given difficulties specifying time of clinical progression), but not the OS analysis, were excluded. Importantly, statistical significance or a trend toward significance was retained at other cut-points as well, including 5%, 10%, and 20% (PFS log-rank p=0.02, p=0.08, and p=0.03, respectively and OS log-rank p=0.003, p=0.01, and p=0.11, respectively; FIG. 8). Notably, an association with HLA-DR expression and response among 13 patients treated with ipilimumab alone was not observed, although the sample size is too small to make definitive conclusions (FIG. 9 and Table 4).

TABLE 4
Clinical characteristics of patients treated with ipilimumab (n = 13).

| | Number | Percentage |
|---|---|---|
| Age | 56 (median) | 34-79 (range) |
| Gender | | |
| Male | 8 | 62 |
| Female | 5 | 38 |
| Stage | | |
| M1a | 1 | 8 |
| M1b | 2 | 15 |
| M1c | 10 | 77 |
| LDH Elevated | 5 | 38 |
| Mutation | | |
| BRAF V600 | 3 | 23 |
| NRAS Q61 | 3 | 23 |
| BRAF/NRAS wild type | 7 | 54 |
| Prior therapies | 0 (median) | 0-3 (range) |
| IL-2 | 0 | 0 |
| Anti-PD-1/PD-L1 | 1 | 8 |
| BRAF +/− MEK inhibitor | 1 | 8 |
| Cytotoxic chemotherapy | 2 | 15 |

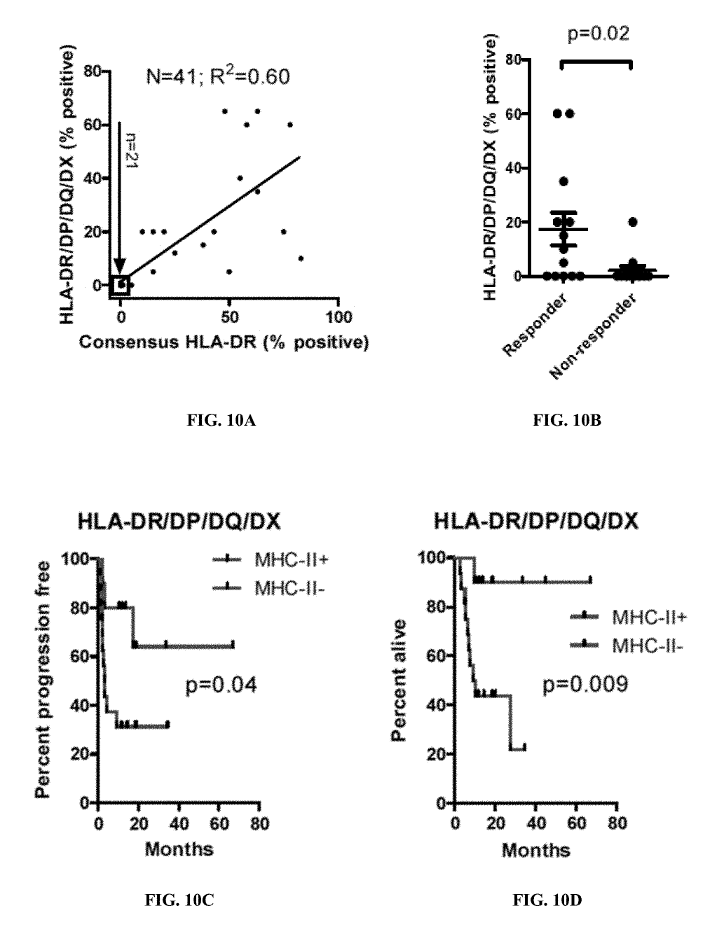

MHC-II antibody specificity and concordance of assessment. To investigate the possibility of alternative MHC class II molecule expression, IHC was performed using a second monoclonal antibody targeting a common epitope to HLA-DR-DP-DQ and -DX (pan-MHC-II) on all samples. Results largely correlated with HLA-DR (FIG. 7E and 10A), supporting high specificity of the HLA-DR antibody. No additional cases were identified as MHC-II(+) by use of the pan-MHC-II antibody. Pan-MHC-II positivity was also associated with objective clinical response (Mann-Whitney's p=0.02, FIG. 10B) as well as PFS and OS using a 5% cut-point (log-rank p=0.04 and p=0.009, respectively; FIGS. 10C-D). Concordance in HLA-DR positivity assessment between two independent blinded pathologists was 77%. After web-mediated discussion of the discordant cases, a final consensus was reached. Concordance and consensus results of the two independent scores for HLA-DR are presented in Tables 5 and 6, respectively.

TABLE 5
Concordance of HLA-DR positivity between two clinical pathologists
blinded to study results (IPI and anti-PD-1/PD-L1 treated patients)

| | | Investigator 2 impression | | | |
|---|---|---|---|---|---|
| | | | | | Not |
| Concordance | | Negative | Positive | Equivocal | evaluable |
| Investigator 1 | Negative | 33 | 7 | 0 | 1 |
| impression | Positive | 5 | 22 | 0 | 0 |
| | Equivocal | 0 | 5 | 0 | 0 |
| | Not | 2 | 1 | 0 | 2 |
| | evaluable | | | | |

TABLE 6
Consensus of HLA-DR positivity between two clinical pathologists
blinded to study results (IPI and anti-PD-1/PD-L1 treated patients)

| Investigator 1 | Investigator 2 | Consensus (# of cases) | | |
|---|---|---|---|---|
| impression | impression | Negative | Positive | Not evaluable |
| Positive | Negative | 2 | 3 | 0 |
| Negative | Positive | 5 | 1 | 1 |
| Equivocal | Positive | 0 | 5 | 0 |
| Not evaluable | Negative | 0 | 0 | 2 |
| Not evaluable | Positive | 0 | 0 | 1 |
| Negative | Not evaluable | 0 | 0 | 1 |

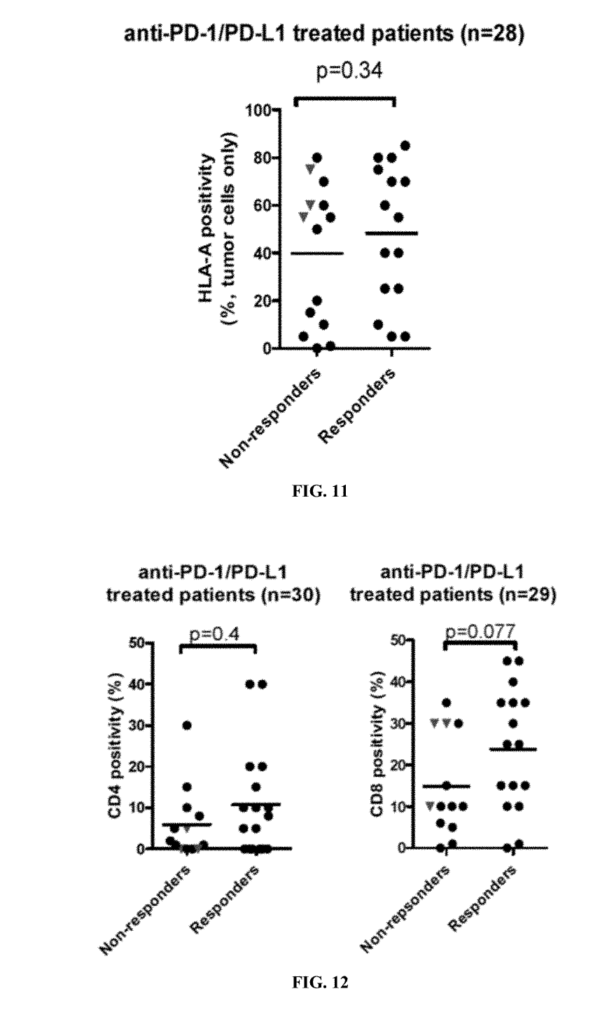

Other Clinical Correlates. To investigate the impact of MHC class I expression on response to anti-PD-1/PD-L1, HLA-A IHC was performed on the same pre-treatment samples. As observed in melanoma cell line models, HLA-A expression was nearly ubiquitous across all tumors and expression level was not statistically associated with response to therapy (FIG. 11). CD4+ and CD8+ T-cell infiltration was also assessed by IHC. CD4 was not statistically associated with therapy response, while a trend toward significance was detected with CD8 (Mann-Whitney's p=0.077; FIG. 12), as has been previously described. The lack of statistical association in the study may be due to scoring method, as the invasive front of the tumor was not detectable in all biopsies or resection specimens. Thus, the total percent positivity of CD8+ T cells invading into the tumor was calculated. Interestingly, the percentage of infiltrating CD4+ T cells were more strongly correlated with HLA-DR expression (Pearson's r=0.63; p=1×10⁻⁵), while CD8+ infiltrate was more weakly correlated (Pearson's r=0.48; p=0.001)(FIG. 7E). Although HLA-DR and CD4+ infiltrate are biologically connected, association of HLA-DR with CD8 infiltrate may be suggestive evidence that enhanced CD4+ Th infiltrate could support the continued accumulation of CD8+ CTLs in the tumor microenvironment. In the instant cohort, PD-L1 immunostaining in the tumor compartment was rare, occurring in 4/24 (17%) tested patients, and showed no correlation with response to PD-1/PD-L1 targeted therapy (FIGS. 13A-C)

### EXAMPLE

Discussion

Targeting the PD-1/PD-L1 signaling axis produces durable responses in a subset of melanoma patients. Although a genetic basis for clinical response to CTLA-4 inhibition in melanoma has recently been suggested, so far few studies have suggested a tumor-cell autonomous basis for response to PD-1/PD-L1 monoclonal antibodies. Herein, a unique inflammatory transcriptional signature in melanoma cell lines that can be identified by tumor cell-specific MHC-II/HLA-DR expression has been identified. Interestingly, heterogeneity in MHC-II expression among panels of melanoma lines has been previously noted. Without wishing to be bound by theory, it is believed that MHC-II expression is either 1) a functional antigen-presenting molecule that can promote CD4 T helper cell aid to the antitumor milieu or 2) a non-functional marker of the inflammatory state of the cell or tumor milieu. The presence of heterogeneity among cell lines grown ex vivo argues against the latter. Yet another alternative hypothesis is that MHC-II expression on melanoma cells could be instrumental in promoting Treg differentiation in a process that requires PD-1/PD-L1 interaction; thus interruption of this signaling could be beneficial in MHC-II+ tumors. Although different CD4 subsets (Th1, Th2, Th17, Treg) were not assessed, superior clinical outcomes with anti-PD-1/PD-L1 therapy was nonetheless observed in patients harboring melanomas with MHC-II expression. A limited analysis of FoxP3 staining in 10 specimens from the cohort with CD4 positivity showed no association of FoxP3 or FoxP3:CD4 ratio with response to PD-1-targeted therapy or with HLA-DR tumor cell positivity (data not shown).

In a bioinformatics analysis of MHC-II expression in melanoma cell lines, which rules out contaminating stromal and immune contribution, a number of gene expression pathways were found to be up-regulated in melanoma cell lines expressing MHC-II (FIG. 1C). The majority of these pathways suggested the presence of an inflammatory signature and reflected gene sets found to be upregulated in response to viral (“WIELAND UP BY HBV INFECTION”), parasitic infections (“KEGG LEISHMANIA INFECTION”), and auto-immune disease (“KEGG GRAFT VERSUS HOST DISEASE”, “KEGG ALLOGRAFT REJECTION”, “KEGG ASTHMA”, and “KEGG AUTOIMMUNE THYROID DISEASE”). Biologically, these pathways reflected stimulation of T-cell receptors (“REACTOME TCR SIGNALING”, and “COSTIMULATION BY THE CD28 FAMILY”) and B-cell activation (“BIOCARTA BLYMPHOCYTE PATHWAY” and “KEGG INTESTINAL IMMUNE NETWORK FOR IGA PRODUCTION”). Although several gene sets were statistically down-regulated in MHC-II(+) cell lines, visual inspection of the heat map suggested that these associations were primarily driven by high expression of target genes in a subset of MHC-II(−) cell lines, specifically Cluster II (FIG. 1C).

Although MHC-I is ubiquitously expressed in most cell types, MHC-II is typically restricted to the immune system, as the MHC-II pathway is thought to utilize extracellular antigens (released from apoptotic or necrotic cells and engulfed by professional APCs). However, tumor-specific MHC-II expression has been noted in a number of malignancies, including breast, colon, and melanoma. Experimentally, MHC-II(+) epithelial cells can present antigen to CD4(+) T-helper cells and enforced expression of MHC-II in tumor cells can promote anti-tumor immunity and tumor rejection in vivo. Collectively these data support a role for aberrant HLA-DR/MHC-II expressing tumors as being a uniquely immunogenic subtype (with the ability to stimulate CD4(+) T-helper cells) which may adapt by expressing PD-L1. Thus, although some MHC-II(-) tumors may express PD-L1, this alone may not permit anti-tumor immunity through PD-1/PD-L1 inhibition.

In this study, HLA-DR expression strongly correlated with response to anti-PD-1. Critically, other relevant variables also co-occurred with HLA-DR expression, demonstrated through in silico cell line analysis (Gene Set Analysis, total somatic mutational burden), flow cytometry of well-characterized melanoma cell lines (PD-L1 expression and CIITA expression), and pre-treatment melanoma samples (CD4 and CD8 T cell infiltration). Together, these data strongly argue that HLA-DR plays a causal or correlative role in anti-PD-1/PD-L1 responses. Interestingly, HLA-A expression did not statistically correlate with CD8 expression in the study (FIG. 7E). This could be due to more ubiquitous expression of HLA-A among the tumors, and it could be that the spectrum of MHC-I neo-antigen may be the rate-limiting step in this association. However, MHC-II expression on the tumor did correlate with CD4 infiltrate, though the nature or composition of these CD4+ cells is not yet understood (Th1, Th2, Th17, or Tregs). Furthermore, in this study, only HLA-A was assessed for MHC-I. Additional contributing effects of HLA-B and HLA-C as well as non-classical MHC-I proteins were not assessed in this study due to limitations in robust antibodies and amount of tissue available for analysis.

Although data point toward a functional role of MHC-II expression as contributing to sensitivity to PD-1/PD-L1 axis inhibition, it is important to note that some tumors responded to PD-1 targeted therapy, despite having no detectable MHC-II expression. There are several possible explanations for this observation: 1) that tumor sampling heterogeneity limited the ability to detect HLA-DR in the tumor and/or 2) that these tumors may be similar to the Ib (Interferon-inducible) group and PD-1 inhibition in these patients may increase CD8 infiltration and local IFNγ secretion, inducing HLA-DR, which could be detected by an on-treatment assessment. Of course, this is hypothetical, and also assumes that HLA-DR is a functional biomarker, rather than a surrogate, which remains to be experimentally proven. Yet a third hypothesis would be that other inflammatory/antigenic factors mediated by MHC-I (such as mutational burden and neo-antigen presence) could be sufficiently high in some cases to circumvent or abrogate an MHC-II requirement. Nonetheless, the potential role of MHC-II as a surrogate biomarker for response cannot be overlooked.

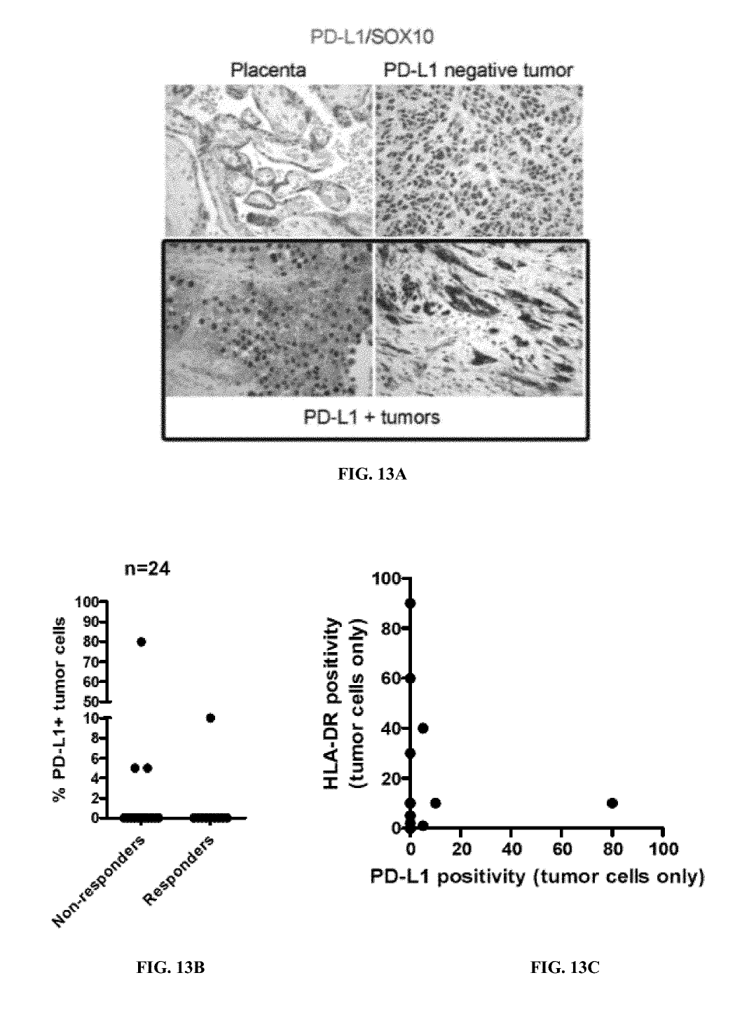

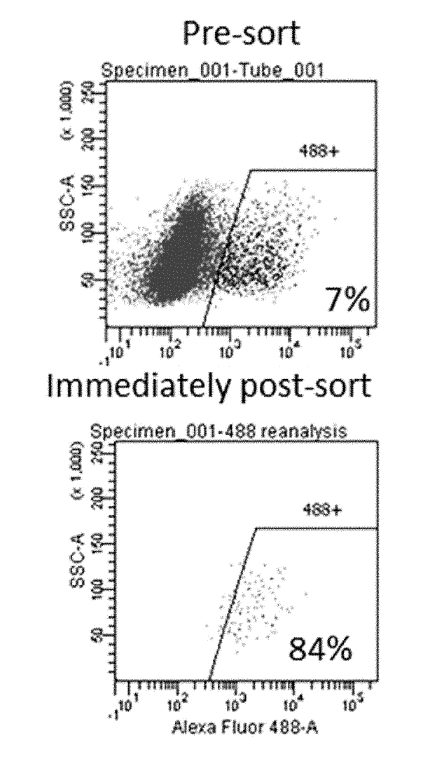

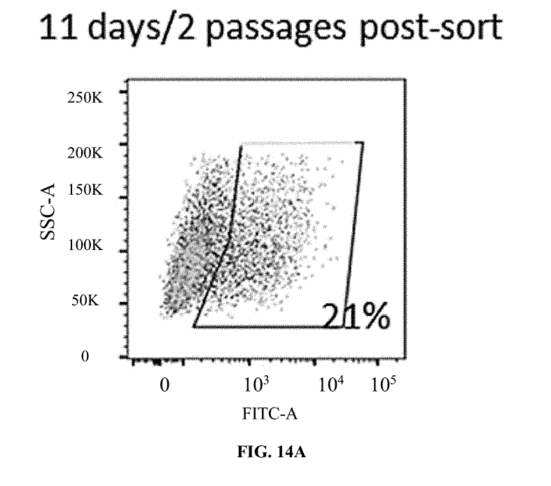

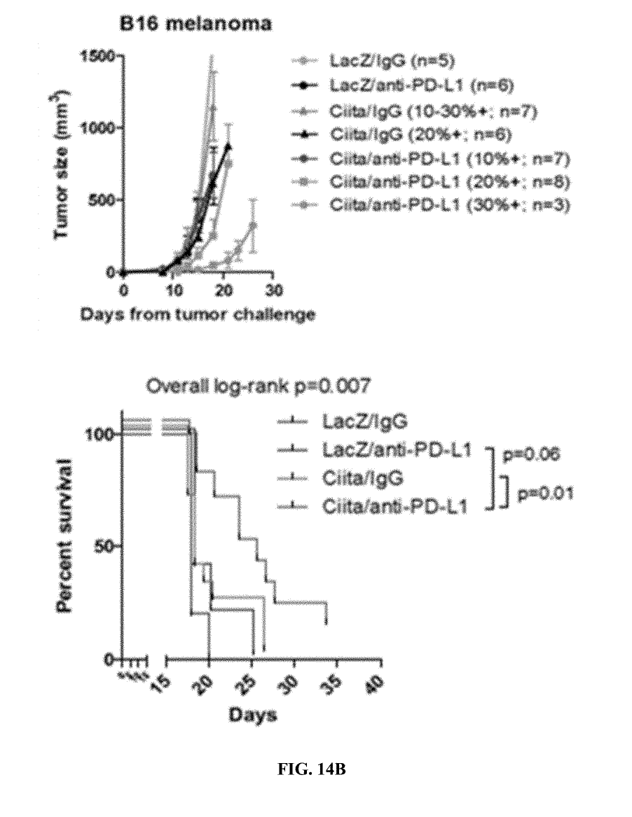

In order to demonstrate a functional role of MHC-II in promoting response to PD-1/PD-L1 therapy, Ciita was overexpressed in B16/F0 melanoma cells to determine whether constitutive tumor cell MHC-II expression would enhance response to PD-L1 mAB in vivo. Despite previous reports of successful constitutive MHC-II (IA/IE) expression by lentivirally-mediated Ciita overexpression, the instant inventors were unable to establish a stable population of MHC-II+ cells in culture, despite repeated rounds of selection and flow sorting (FIG. 14A). Expansion of the positive population in cell numbers sufficient for the experiment routinely caused the MHC-II+ population to degrade to near 1-2% after 3-5 passages. The reason for this selection is presently unclear but is a matter of current investigation. Possible explanations are silencing of the lentiviral promoter or cell-mediated internalization of MHC-II.

Nonetheless, either control (LacZ-expressing) or Ciita/MHC-II+ B16 cells (ranging from 10-30% MHC-II+ at the time of injection) was injected into the flank of C57/B16 mice and monitored tumor growth and survival with either IgG (isotype) control or anti-PD-L1 mAB, given twice weekly, beginning on day +1 following tumor cell challenge. The subgroup of Ciita+ B16 melanoma cells with the highest degree of MHC-II positivity (30%) at the time of injection, treated with anti-PD-L1, had slower tumor formation and prolonged survival, although the effect was marginal (FIG. 14B). Without wishing to be bound by theory, it is believed that the observed effect may not have been robust due to unstable expression and rapid selection of Ciita-transduced cells in vitro and in vivo. Interestingly, there appears to be an MHC-II+ dose-effect in response to PD-L1 mAB (i.e. 30% MHC-II+ responded better than 10 or 20%). While these results are difficult to interpret due to difficulty in establishing a pure cell line, they are believed to support a potential functional role of MHC-II expression in immunotherapy response.

Conflicting reports of stromal versus tumor PD-L1 staining, coupled with lack of standardization, proprietary nature, and the difficulties associated with PD-L1 as an IHC antigen have precluded the routine use of this marker in the clinic. In the study, a relatively low number of samples stained positively for PD-L1, despite appropriate positive controls (human placenta). The low proportion of samples with PD-L1 staining and lack of correlation of positivity with patient benefit reinforce the problems of using PD-L1 as a clinical biomarker. In contrast, HLA-DR can be robustly identified on tumor cells through use of dual-color IHC using well-established commercially available antibodies. Thus, it is proposed that with additional validation, melanoma HLA-DR expression may be a rapidly translatable biomarker for patient stratification of PD-1/PD-L1 immunotherapy which can easily be performed in standard pathology laboratories at most institutions at low cost. This marker, if validated, could be envisioned to stratify patients toward anti-PD-1 monotherapy and away from the more toxic but potentially more clinically-active combination of ipilimumab and nivolumab. Furthermore, understanding the biological basis for differential MHC-II expression among melanomas may identify agents that induce MHC-II positivity and can be used in combination with PD-1/PD-L1 targeted therapy to enhance response rates.

Methods

Immunoblotting was performed as previously described³² Briefly, cells were washed in cold phosphate-buffered saline, collected and lysed in 1×RIPA buffer (50 mM Tris (pH 7.4), 1% NP-40, 150 mM NaCl, 1 mM EDTA, 0.1% SDS, 0.25% sodium deoxycholate, 5 mM NaF, 5 mM Na3VO4, 10% glycerol, 1M phenylmethyl-sulphonylfluoride and protease inhibitors) for 30 min on ice. Lysates were sonicated for 2-3 s to shear DNA and cleared by centrifugation at 13,200 r.p.m. for 15 min. Protein concentrations of the lysates were determined by BCA assay (Bio-Rad, Hercules, Calif.). Samples were separated by SDS-PAGE and transferred to nitrocellulose membrane. Membranes were blocked with 5% non-fat dry milk or 5% bovine serum albumin in tris-buffered saline with 0.1% Tween-20 for 1 h at room temperature and then incubated overnight at 4° C. with the appropriate antibody as indicated. Following incubation with appropriate horseradish peroxidase-conjugated secondary antibodies, proteins were visualized using an enhanced chemiluminescence detection system. This study was performed using the following antibodies: p-STAT1 (Cell Signaling Technology. #7649, 1:5000) STAT1 (Santa Cruz Biotechnology. # SC592. 1:5000), p-ERK1/2 (Cell Signaling Technology #9101, 1:5000), ERK1/2 (Cell Signaling Technology #9102. 1 5000), CIITA (Cell Signaling Technology #3793, 1:1000) HLA-DR (Santa Cruz, sc-53319., 1:5000).

Standard Flow Cytometry. Flow cytometry was performed using the following antibodies: HLA-DR/PE-Cy7 (Biolegend, clone L243. 1:20). CD274/PD-L1/APC (Biolegend, clone 29E.2A3, 1:200) and HLA-A/B/C-Alexa Fluor488 (1:100, Biolegend, clone W6/32) mouse MHC-II (I-A/I-E 1:20 Biolegend, clone M5/114.15.2). DAPI was used as a viability dye. Samples were analyzed on an Aria III laser system (BD Biosciences)

Phospho-flow cytometry. Melanoma cell lines were treated with Accutase™ (EMD Millipore, # SCR005) for 10 minutes at 37° C. to dissociate them from the plate. Dissociated cell lines were rested at 37° C. in a CO2 incubator for 30 minutes prior to stimulation. After resting, cells were stimulated by adding IFNy (Cell Signaling) at a final concentration of 100 ng/mL. During signaling, cells were kept in a 37° C. CO2 incubator. After 15 minutes of signaling, cells were fixed for 10 minutes at room temperature with a final concentration of 1.6% paraformaldehyde (Electron Microscopy Services). Cells were then pelleted and permeabilized by resuspension in 2 ml of methanol and stored over night at −20° C. Flow cytometry was performed using the following antibodies: HLA-DR/BV421 (BD Horizon™, clone G46-6, 1:40), p-STAT5/PE-Cy7 pY694 (BD Phosflow™, clone 47, 1:10), and p-STAT1/PerCP-Cy5.5 pY701 (BD Phosflow™, clone 4A, 1:10). Samples were analyzed on a LSRII system (BD Biosciences).

Immunohistochemistry. For HLA-DR (Santa Cruz [sc-53319], 1:1000)/SOX10 (LsBio [LS-C312170], 1:30), HLA-DR-DP-DQ-DX (Santa Cruz [sc53302), 1:1000)/SOX10, HLA-A (Santa Cruz [sc-365485], 1:1300)/SOX10, and PD-L1 (Cell Signaling #13684, 1:500)/SOX10 dual IHC tumor sections were stained overnight at 4° C. with both antibodies. Antigen retrieval was performed using Citrate Buffer (pH 6) using a Biocare Decloaking Chamber. The visualization system utilized was MACH2 (Biocare) using DAB (Dako) and Warp Red (Elmore), and counterstained with hematoxylin.

For CD4 and CD8 staining, slides were placed on a Leica Bond Max IHC stainer. All steps besides dehydration, clearing and coverslipping are performed on the Bond Max. Heal induced antigen retrieval was performed on the Bond Max using their Epitope Retrieval 2 solution for 20 minutes. Slides were incubated with anti-CD4 (PA0427, Leica, Buffalo Grove, Ill.) or anti-CD8 (MS-457-R7, ThermoScientific. Kalamazoo. Mich.) for one hour. The Bond Polymer Refine detection system was used for visualization. CD4 and CD8 were scored as % infiltrating CD4(+) or CD8(+) cells in the tumor area.

HLA-DR scoring determination. Two pathologists (MVE and RS) who were unaware of clinical response data made independent visual estimations of the percentage of tumor membrane-specific positivity for HLA-DR, in SOX10(+) nuclei areas, in the whole tumor section focusing at the tumor hot spots. For all staining batches positive and negative controls (human tonsil; HLA-DR is positive in germinal and non-germinal center cells and negative in squamous epithelial cells) were included and stained appropriately and reproducibly in all cases. Furthermore, nearly all cases had positive-staining stromal cells (presumably B-cells and macrophages) as an internal control. In concordant cases (both investigators scored as ‘negative’ (1% or less of all tumor cells in the entire tissue section staining positive; i.e. all analyzable fields of view) or ‘positive’ (>1% of tumor cells in the entire tissue section staining positive; i.e. all analyzable fields of view)), the result was averaged. For discordant cases {i.e. positive vs. negative interpretation, or any concerns on evaluable nature of the specimen) the investigators reviewed the case together to reach a final conclusion or consensus. If no consensus could be agreed upon, the sample was listed as non-evaluable.

Cancer Cell Line Encyclopedia analysis. Gene expression data (Affymetrix hg133p1us2) from the Cancer Cell Line Encyclopedia (CCLE) were downloaded from the Broad Institute (broadinstitute.org) and analyzed in R (r-project.org/). RMA-normalized melanoma cell line data were collapsed to the gene level and filtered using the ‘genefilter’ package. Differentially expressed genes were identified using a t-test with a false-discovery rate correction. Hierarchical clustering was performed using 1-Spearman's rank correlation and complete linkage. Gene Set Analysis was performed using the GSA package in R and the maxmean statistic. Gene sets in the molecular signatures database curated gene sets C2 collection (version 3.0) were utilized for GSA.

Cell and tumor culture. SKMEL-28 and WM115 cell lines were obtained from Dr. Kimberly Dahlman (Vanderbilt University), CHL-1 and HMCB melanoma cell lines were obtained from the laboratory of William Pao (Vanderbilt University). Cell line nature was not directly authenticated, but protein marker expression was consistent with published HLA-DIM mRNA expression patterns (CCLE). Cell lines were confirmed mycoplasma-free and cultured in DMEM containing 10% FBS. Stimulation with recombinant human IFNγ (R&D Systems) was performed at 100 ng/mL. For PDX models and ex-vivo organotypic culture, tumors were freshly resected and sectioned using an Alto tissue matrix sectioner (Roboz Surgical, Gaithersburg, Md.).

Patients. Patient samples and data were procured based on availability of tissue and were not collected according to a pre-specified power analysis. All patients were consented on IRB approved protocols (Vanderbilt IRB #030220 and 100178). Tumor samples for the TMA and for the HLA-DR staining cohort were obtained from tumor biopsies or tumor resections obtained for clinical purposes. Samples were obtained within 2 years of start of anti-PD-1/PD-L1 therapy (nivolumab, pembrolizumab, MPDL3280a). Only patients with available tumor samples and evaluable responses were included. In cases where multiple tissues were available for the same patient, the evaluable sample collected closest to PD-1 therapy was utilized for scoring. Clinical characteristics and objective response data were obtained by retrospective review of the electronic medical record. All responses were investigator assessed, RECIST defined responses or (in a single case) prolonged stable disease with clinical benefit lasting >3 years.

For the validation set, all patients were consented to an IRB-approved tissue banking protocol (for MGH patients as part of either Dana Farber Harvard Cancer Center protocols 02-017 and 11-181). Samples were obtained prior to therapy with anti-PD-1/PD-L1 monoclonal antibodies for research (as opposed to clinical) purposes. A linked database was prospectively maintained and regularly updated with clinical characteristics, response to therapy, date of progression (if applicable), and date of death or last follow up visit.

Statistical analysis. The tests of hypotheses concerning between two groups comparisons were completed using either two-sample Student t-test or non-parametric Wilcoxon rank sum test for continuous variables of interest. The Analysis of Variance (ANOVA) with Tukey's multiple comparison adjustment was used for comparisons of more than two independent groups. Dichotomous data were compared using the chi-square test ith the Yates correction or Fisher's exact test when appropriate. The Kolmogorov-Smirnov test (KS-test) was used to determine if the distribution of the datasets differed significantly. For progression free survival (PFS) analysis, the survival curves were estimated using the Kaplan-Meier method with the log-rank test to examine the statistically significant differences between study groups. For gene analysis, the FDR adjusted Student t-test was used to identify the “winner genes” then followed by the complete linkage cluster analysis based on 1-Spearman correlation. Statistical analyses were performed using R or GraphPad Prism. All P values reported were 2-sided.

### Example 2

Reduced tumor lymphocytic infiltration in the residual disease (RD) of post-neoadjuvant chemotherapy (NAC) triple-negative breast cancers (TNBC) is associated with Ras/MAPK activation and poorer survival.

Background: Tumor-infiltrating lymphocytes (TILs) are associated with improved prognosis in TNBCs, with several retrospective analyses demonstrating that TNBCs with high baseline TILs have higher rates of pathologic complete response (pCR) to NAC. Moreover, the TIL burden in the RD of patients who do not achieve pCR to NAC is also correlated with prognosis. However, insight into the molecular pathways in TNBC which modulate heterogeneity in host anti-tumor immune responses is lacking. To address this gap in knowledge, TILs were analyzed retrospectively in a cohort of clinically and molecularly characterized TNBCs with RD after NAC.

Methods: TILs were scored in H&E stained slides by expert pathologists in the post-treatment tumors of 92 NAC-treated TNBC patients with RD at the time of resection and in 44 matched baseline diagnostic biopsies. Genomic alterations in the RD were assayed using targeted next-generation sequencing (tNGS) while selected transcriptional signatures were evaluated by NanoString as previously published (Balko et al, Cancer Discovery 2014). Differences in pre- and post-NAC TILs were compared between tumors harboring alterations in cell cycle, PI3K/mTOR, growth factor receptors, Ras/MAPK and DNA repair pathways. Associations of TILs with transcriptional signatures were also tested.

Results: A strong positive association of TILs in NAC-treated specimens was observed with RFS (coxPH p=0.0001, relative risk reduction of 3.4% for each % of TILs) and OS (p=0.0016; relative risk reduction of 2.8% for each % of TILs). In multivariate analysis with stage, age, node status and RD tumor cellularity, TILs in the post-NAC disease remained a significant predictor of RFS and OS (p=0.0008 and p=0.007, respectively). TILs tended to decrease with NAC in paired samples, although this decrease was not statistically significant (p=0.07).

Genetic alterations in the Ras/MAPK (amplifications in KRAS, BRAF, RAFT and truncations in NF1) and cell cycle pathway (alterations in CCND 1-3, CDK4, CDK6, CCNE1, RB, AURKA and CDKN2A) were associated with lower TILs in RD (p=0.005 and p=0.05, respectively). A significant inverse linear correlation was detected between a transcriptional signature of Ras/MAPK activation (Pratilas et al, PNAS 2009) and TILs in the RD (Spearman's r=−0.42; p=0.00028). Total number of alterations of likely functional significance detected by tNGS showed no association with TILs, suggesting that the association of Ras/MAPK deregulation and cell cycle alterations with TILs may be a pathway-specific effect.

In TNBC cell lines, chemical inhibition of MEK transcriptionally up-regulated MHC-I and MHC-II molecules, while simultaneously down-regulating mRNA expression of the immune checkpoint inhibitor PD-L1 (MDA-231 p=0.00002, BT549 p=0.0003, and SUM159PT p=0.009). In vivo experiments confirming these associations are underway.

Conclusions: The data suggest a strong correlation of Ras/MAPK pathway activation with immune-evasion and outcome in TNBC. With additional mechanistic understanding, rational design of clinical trials combining MEK inhibitors with PD-L1 antibodies in TNBC may be warranted.

### Example 3

Preliminary data for use of tumor membrane-specific HLA-DR expression as a biomarker of response to PD-1/PD-L1 directed therapy.

Goal: To determine the rate of prediction of tumor cells expressing HLA-DR on response to PD-1/PD-L1 directed therapy.

Methods: 12 sections from excisional biopsies or surgical resections of melanoma were immune-stained for HLA-DR (TAL-1B5, commercially available for research from multiple vendors). These 12 sections represented 11 patients; 5 responders to anti-PD-1/PD-L1 therapy and 6 non-responders. Two samples were from sequential biopsies, one from prior to a clinical response, and one upon acquisition of resistance (relapse) on therapy.

Tumor sections were stained overnight at 4 C at a 1:1000 dilution. Antigen retrieval was performed using Citrate Buffer (pH6) using a Biocare Dechloaking Chamber. The Visualization System utilized was Envision-Mouse using DAB chromogen and counterstained with Hematoxylin.

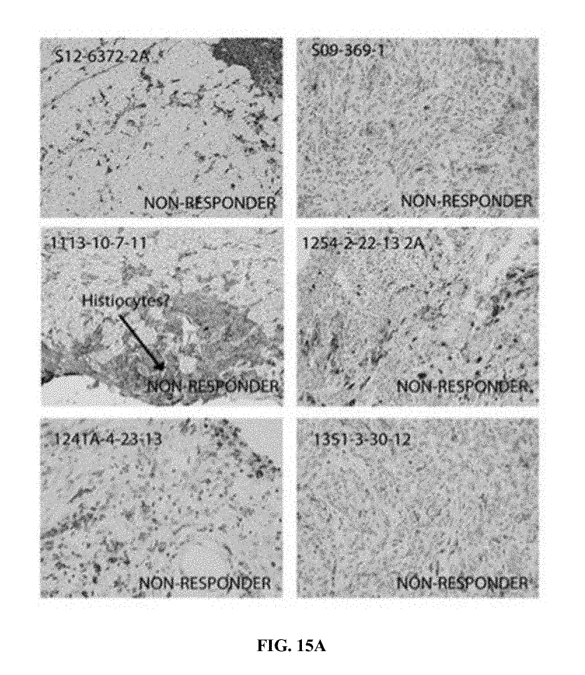

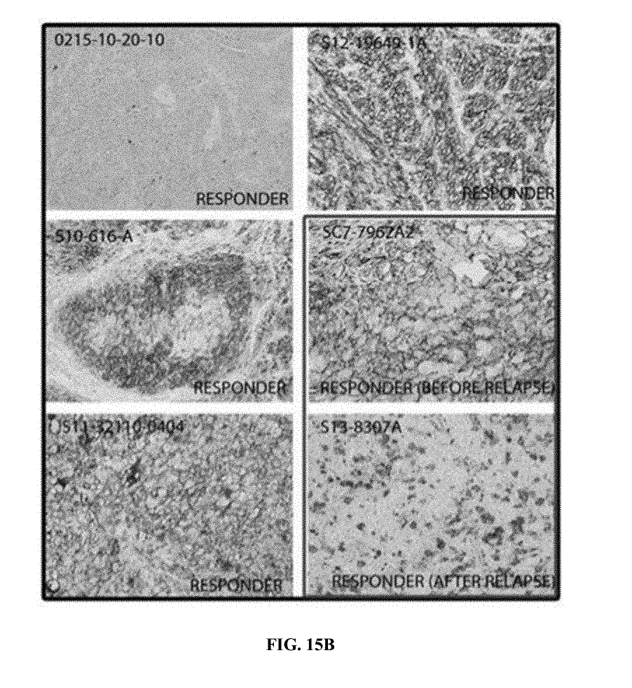

Results: Of 6 non-responders, 0/6 exhibited conclusive tumor-specific membrane staining of HLA-DR (FIG. 15A). One sample (1113-10-7-11) had regional edge cells that stained positive, but were considered likely to be histiocytes and not tumor cells by the pathologist. Of 5 responders, 4/5 had high membrane specific staining of HLA-DR on what appear to be tumor cells (FIG. 15B). Dual staining for melanoma-specific markers are being conducted to confirm the staining pattern. Of note, the one responder sample that was negative for HLA-DR (0215-10-20-10) was from a previous resection several years before therapy and may not be representative of the on-therapy disease. Analysis of sequential samples (prior to response[SC7-7962A2], and after relapse on therapy [S13-8307A]) suggested loss of HLA-DR on the tumor cells that coincided with acquired resistance.

Conclusion: HLA-DR expression on the tumor seems to be a useful biomarker for prediction of response to PD-1/PD-L1 targeted therapy.

### Example 4

Formalin fixed paraffin embedded melanoma tumor sections were stained with anti-HLA-DR antibody and anti-SOX10 antibody and reviewed by a pathologist for dual positive tumor cells. Two sample sets were stained independently comprising a total of 35 patients. Patients were then classified by their clinical response to targeted immunotherapy, where known. 32 patients were evaluable, with 3 additional considered equivocal due to uncharacteristic features of HLA-DR staining or lack of SOX10 staining in the perceived tumor region (FIG. 16). PR and CR refer to partial and complete response, and PD refers to progressive disease.

### Example 5

Melanoma-specific MHC-II expression predicts response to α-PD-1 therapy.

Background. αPD-1 therapy yields objective clinical responses in 30-40% of advanced melanoma (MEL) patients. While promising, many patients do not benefit clinically. As such, predictive biomarkers to guide patient selection are needed. A number of predictive biomarkers have been suggested in the literature, including tumor or immune cell expression of PD-L1, identification of neo-antigens through next generation sequencing techniques, and T-cell receptor sequencing. While quite promising, these assays are technically challenging and require specialized tissue processing or bioinformatics.

Methods. MHC-I/II mRNA was profiled across 60 MEL cell lines. The transcriptional characteristics of MHC-II+ cell lines were analyzed by Gene Set Analysis. Cell surface expression of MHC-I and MHC-II was confirmed by flow cytometry (FC) in a subset of cell lines under basal and stimulated (IFNγ) conditions. In 26 tumor samples from αPD-1 treated MEL patients, immunohistochemistry (IHC) was performed for HLA-DR (MHC-II) or HLA-A (MHC-I), SOX10, CD4 and CD8. IHC results were correlated with response and progression-free survival (PFS).

Results. MHC-I mRNA was expressed in all cell lines while MHC-II expression was bimodal (60% positive). MHC-Ir cell lines had transcriptional signatures of the PD-1 signaling, allograft rejection, and T-cell receptor signaling. By FC, MHC-II⁺ (mRNA) cell lines were constitutive and inducible (IFNγ stimulation) for HLA-DR while MHC-II⁻ cells did not express or induce HLA-DR. In contrast, all tested cell lines significantly upregulated PD-L1 with IFNγ stimulation. Of 26 patients treated with αPD-1, 10 were MHC-II⁺. All 10 MHC-II⁺ (100%) patients had partial, complete, or mixed responses (MR), while only 7/16 (44%) of MHC-II⁻ patients benefited (Fisher's exact p=0.004). Excluding MR patients (n=2), median PFS for MHC-II⁺ was 728 days, while the median PFS for MHC-II⁻ tumors was 98 days (log-rank p=0.01). MHC-II⁺ tumors had enhanced CD4 and CD8 infiltrate (Pearson's correlation p=0.000002 and p=0.03, respectively). MHC-I positivity was ubiquitous and not associated with response.

Conclusions. A subset of MEL demonstrates an MHC-II signature that correlates with αPD-1 response and enhanced CD4/CD8 T-cell infiltrate. Without wishing to be bound theory, this is believed to indicate that tumor antigen presentation (MHC-II expression) is a requirement of αPD-1 benefit, and presence of these cell surface markers is predictive benefit. MHC-II+ tumors can be robustly identified by routine melanoma-specific IHC for HLA-DR to guide patient selection. Combining HLA-DR IHC with other biomarkers, including PD-L1 expression may further improve patient selection.

### Example 6

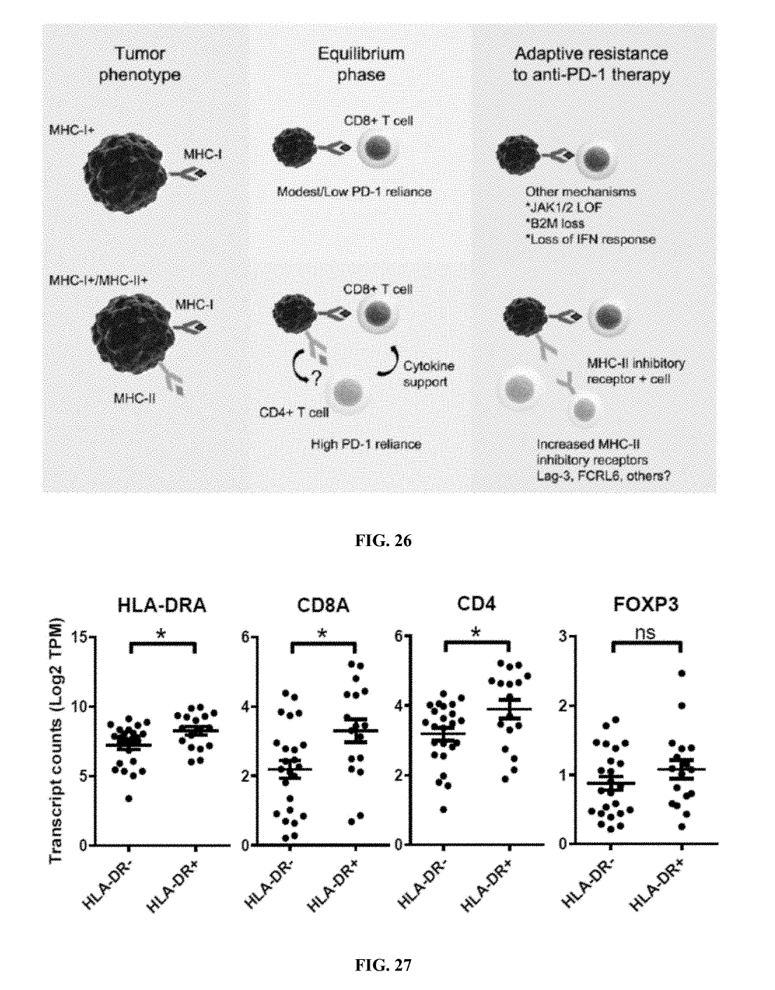

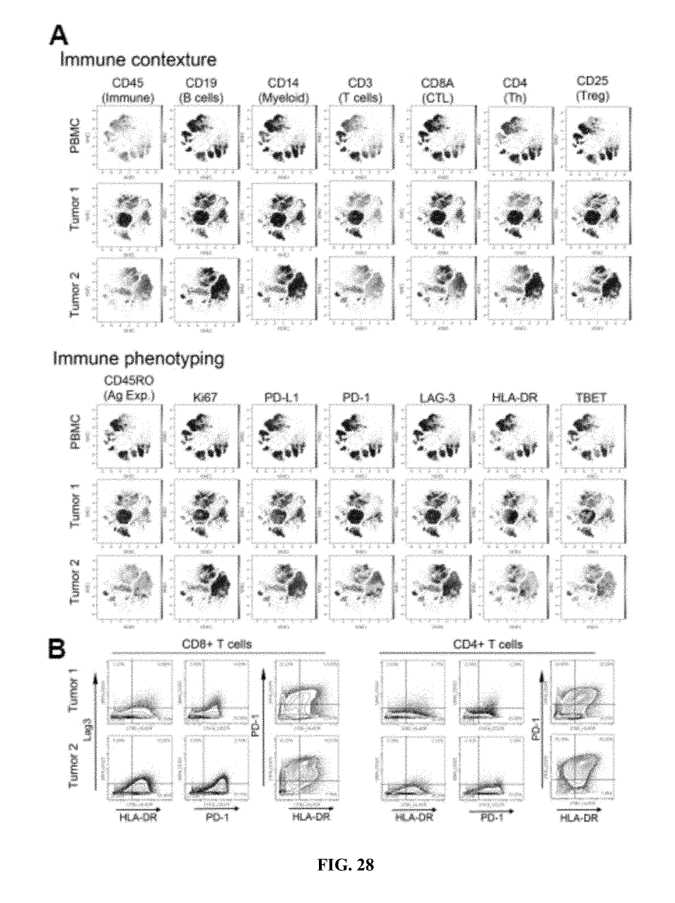

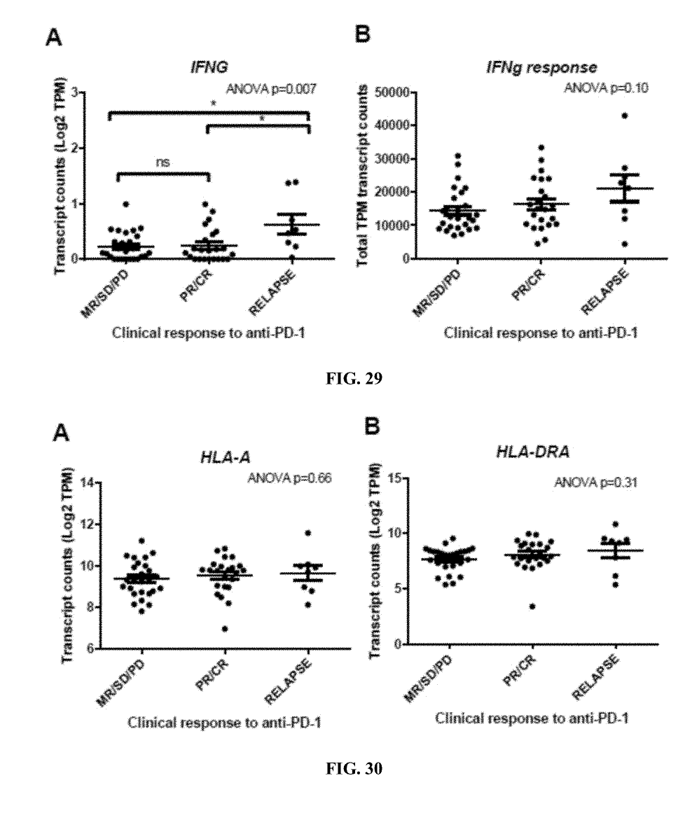

MHC-II+ tumors are enriched with gene expression patterns of adaptive immunity. Melanomas with constitutive tumor cell-autonomous MHC-II/HLA-DR expression are associated with high CD4 and CD8 infiltration and enhanced responses to PD-1-targeted immunotherapy (59, 62), a finding subsequently confirmed by others (61). Furthermore, MHC-II+ melanoma cell lines (grown in the absence of stroma or IFN-γ-expressing cells) demonstrate intrinsic gene expression patterns of inflammation and autoimmunity (59). However, HLA-DR expression by melanoma cells in vivo could be due to membrane exchange (trogocytosis) in an inflammatory milieu (63). To confirm that HLA-DR is endogenously expressed by tumor cells, dual RNA-in situ/IHC analysis for CIITA and HLA-DR, respectively, was performed on melanoma specimens. As shown in FIG. 26 MHC-II/HLA-DR+ tumor cells expressed mRNA for CIITA, the master regulator of MHC-II gene transcription, suggesting this is a tumor cell-autonomous phenotype in human melanoma.

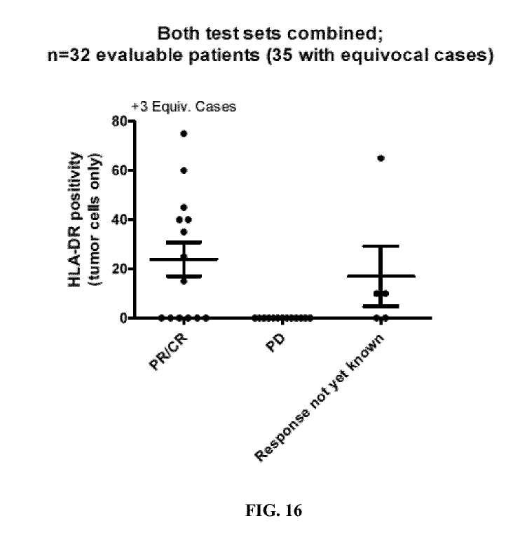

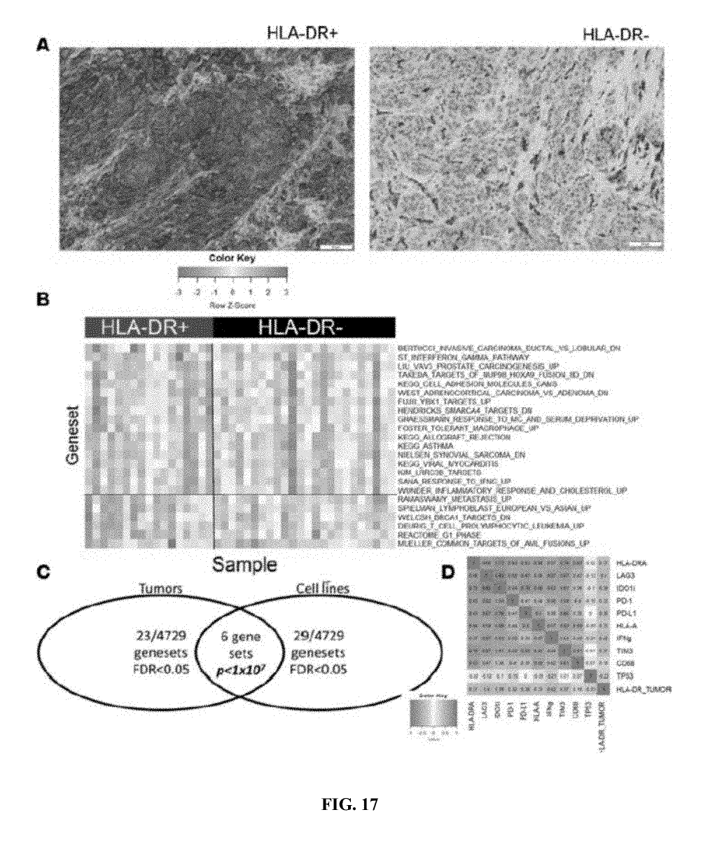

RNA-sequencing analysis was performed on a series of anti-PD-1-treated melanoma and non-small cell lung cancers (n=58, including 50 pre-anti-PD-1 samples and 8 samples obtained after anti-PD-1 following acquired resistance) and scored tumor-specific HLA-DR expression by IHC (HLA-DR staining available on 41 of 58; FIG. 17A) prior to their treatment with PD-1-targeted immunotherapy. Tumors with at least 5% of tumor cells expressing cell-surface HLA-DR demonstrated similar gene set enrichment as observed in the previously published analyses of melanoma cell lines (59). The gene sets enriched (FDR<5%) in HLA-DR+ tumors included those associated with allograft rejection, viral myocarditis, autoinflammatory disease (asthma), and IFN-γ response pathways (FIG. 17B). Although HLA-DR is an IFN-γ-inducible gene, the previous studies performed on cultured tumor cell lines (without IFN-γ) suggested that this finding is likely linked, at least partially, to the intrinsic state of the tumor cells, rather than a direct measure of IFN-γ activity in the microenvironment. This is supported by a high degree of overlap between enriched gene sets in MHC-II+ human tumors and cultured cell lines (in the absence of IFN-γ) identified in this study and the previous work (59) (FIG. 17C). HLA-DR+ tumors had greater mRNA expression of MHC-II genes, such as HLA-DRA, although the association was weak, reflecting the independent contribution of HLA-DR+ stroma (e.g., macrophages, dendritic cells) to this measurement. In addition, consistent with the prior IHC observations, HLA-DR+ tumors had higher CD8A and CD4 expression, without enhanced regulatory T cell markers, such as Foxp3 (FIG. 27) MHC-II+ tumors are associated with higher expression of immune checkpoint receptors.

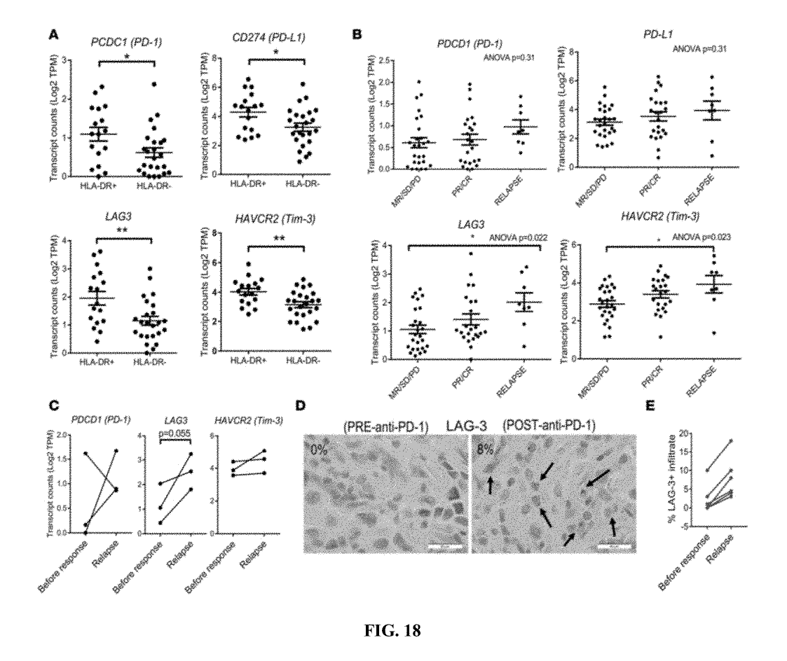

To explore the effects of tumor cell-autonomous MHC-II expression on antigen presentation machinery and immune checkpoints, HLA-DR expression was correlated (scored by IHC) with genes associated with MHC-II (HLA-DRA), MHC-I (HLA-A), T cell repression (PD-1/PDCD1, PD-L1/CD274, IDOL TIM-3/HAVCR2, and LAG3), T cell activation (IFNG), monocyte infiltration (CD68), and a ubiquitous marker (TP53) as a control (FIG. 17D). The expression of most immune-related genes were positively correlated with one another. HLA-DR IHC expression, as scored only in the tumor compartment, also correlated with most immune genes, but less strongly with the myeloid marker CD68. Interestingly, all examined immune checkpoint receptor genes also highly correlated with HLA-DR positivity, and the most significant of these was LAG3. These associations were also evident when stratifying tumors by MHC-II+≥5% (FIG. 18A). Of particular interest in this analysis was the association of HLA-DR tumor cell positivity with LAG3, which competes with CD4 as a ligand for MHC-II, thereby suppressing MHC-II-mediated antigen presentation (64, 65).

To determine what cell types in the melanoma microenvironment express LAG-3, was performed mass cytometry (CyTOF) on two human patient melanoma resections as well as PBMCs from a healthy individual. viSNE analyses of resected melanomas demonstrated the following observations (FIG. 28A). LAG-3 was exclusively expressed by T cells, primarily CD8+ T cells, but much less so by CD4+ cells. LAG-3+ cells were a less abundant subset of PD-1+ T cells, which were found primarily on both CD4+ and CD8+ antigen-experienced (CD45RO+) and effector (TBET+) cells in the tumor microenvironment. A subset of PD-1+ cells was also Ki67+ (cycling). However, LAG-3 appeared to be exclusive of Ki67 positivity, possibly reflecting a more senescent phenotype. LAG-3 was not detected on CD25+CD4+ cells, suggesting its dissociation from a classical T regulatory phenotype. Interestingly, although neither tumor expressed abundant MHC-II (HLA-DR), MHC-II was highly expressed by B cells and a substantial fraction of PD-1+ T cells that also appeared to overlap with LAG-3 expression (FIG. 28B).

Next, the association between gene expression of checkpoint molecules and ligands with annotated clinical response to anti-PD-1 in these patients was examined. Included in this analysis were 49 pretreatment tumors as well as tumor samples available from patients who initially responded to anti-PD-1 therapy but subsequently progressed (i.e., relapsed) (n=6 patients and n=8 samples, with 3 isolated resections/biopsies from a single patient). When comparing treatment response groups, LAG3 and HAVCR2 (encoding Tim-3) showed differential expression by ANOVA. Of interest, neither LAG3 nor HAVCR2 expression obviously correlated with intrinsic resistance (in responding vs. nonresponding patients), but they were significantly higher in progression (relapse) specimens (i.e., acquired resistance) (FIG. 18B). IFNG expression was also higher in relapsed patients. Although an IFN-γ-response signature was not elevated, it showed a similar trend, suggesting that the majority of these tumors had not lost IFN-γ activity or expression (Supplemental FIG. 4). Neither transcript expression of MHC-I (HLA-A) nor MHC-II (HLA-DRA) was associated with clinical responses to PD-1 in patients, stressing the unique information gained by examining MHC-II protein expression by IHC specifically in tumor cells (59) (FIG. 30). Finally, the cohort included 3 patients in whom paired tumor samples were available both prior to response to anti-PD-1 and at relapse/progression. When considering checkpoint molecule expression (PDCD1, LAG3, and HAVCR2) in these 3 matched pairs, both LAG3 and HAVCR2 demonstrated a trend toward enrichment in 3 of 3 specimens (1-tailed t test P =0.15), although enrichment of LAG3 was more striking (1-tailed t test P=0.055; FIG. 18C). IHC analysis for LAG-3+ tumor-infiltrating lymphocytes (TILs) confirmed this trend (n=5 pairs, before and at acquired resistance, all unique patients; FIGS. 18, D and E). Upon resistance to anti-PD-1, increased LAG3 and HAVCR2 have been observed in both humanized murine models (66) and patients (51).

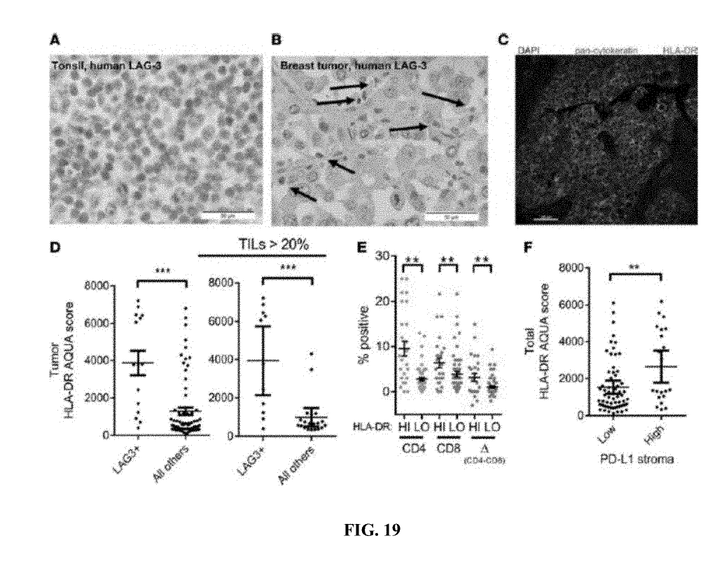

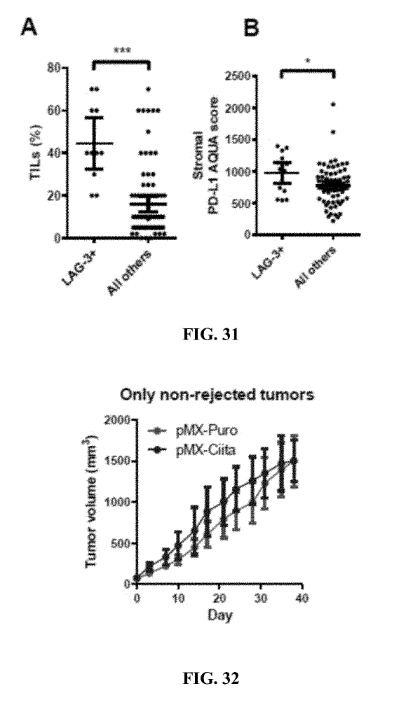

Association of MHC-II expression with inflammation and LAG-3 expression in breast cancer. Tumor cell-autonomous MHC-II expression as an important biomarker in breast cancer was previously identified. MHC-II+ breast tumors were found to have a greater degree of TILs after neoadjuvant chemotherapy, which correlates with improved outcomes after surgical resection (67). Since this series of 112 triple-negative breast cancers (TNBCs) were previously molecularly characterized for MHC-II/HLA-DR expression in the tumor compartment (67), to determine whether a similar association of MHC-II+ tumors with LAG-3+ TILs (FIGS. 19, A and B) could be observed in this cohort. HLA-DR was scored using automated quantitative analysis (AQUA; FIG. 19C*). A strong association of the presence of LAG-3⁺ TILs in tumors with high HLA-DR expression, both across the entire series and after controlling for the intrinsically higher rate of TILs in HLA-DR+ tumors by including only heavily infiltrated (TILs >20%) tumors in the analysis (FIG. 19D and FIG. 31) was found. Consistent with findings in melanoma (59), HLA-DR+ tumors were also strongly associated with the presence of CD4 and, to a lesser degree, CD8 infiltrates. However, the enrichment for CD4+ T cells in the HLA-DR⁺ tumors was significantly higher (P<0.01) (FIG. 19E). HLA-DR positivity also correlated with higher PD-L1 expression in the tumor-associated stroma, but the association of PD-L1+ stroma with LAG-3+ TILs was weaker than that of HLA-DR, suggesting that HLA-DR positivity is associated with LAG-3+ TILs and higher PD-L1 expression in the tumor microenvironment and that LAG-3 positivity and PD-L1 positivity are not necessarily directly correlated (FIG. 19F and FIG. 31B).

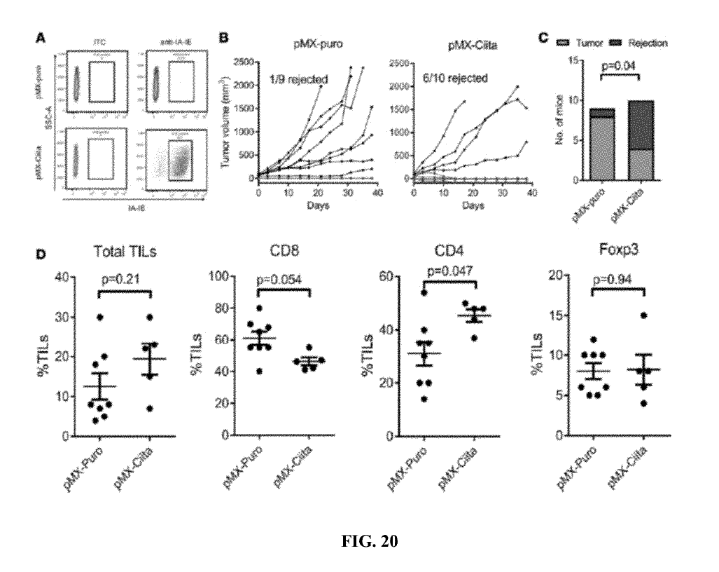

Enforced expression of MHC-II promotes tumor rejection, CD4+ T cell recruitment, and a specific pathway to immune evasion. To better understand the direct role of MHC-II expression on the tumor microenvironment, expression of MHC-II on MMTV-neu breast tumor cells was enforced through transduction of Ciita, the master regulator of MHC-II. Cells transduced with Ciita were strongly IA-IE+ (murine MHC-II), by flow cytometry analysis (FIG. 20A). When equivalent CIITA+ or vector control cells were injected orthotopically into the mammary fat pads of wild-type FVB/n mice, a substantially greater rejection rate was observed (FIGS. 20, B and C). Rejecting mice were 100% resistant to rechallenge (data not shown). In mice that did form tumors, the tumor growth rate was similar in both MHC-II+ and MHC-II− tumors (FIG. 32), suggesting a robust adaptive resistance to the presence of tumor-autonomous MHC-II in a subset of tumors.

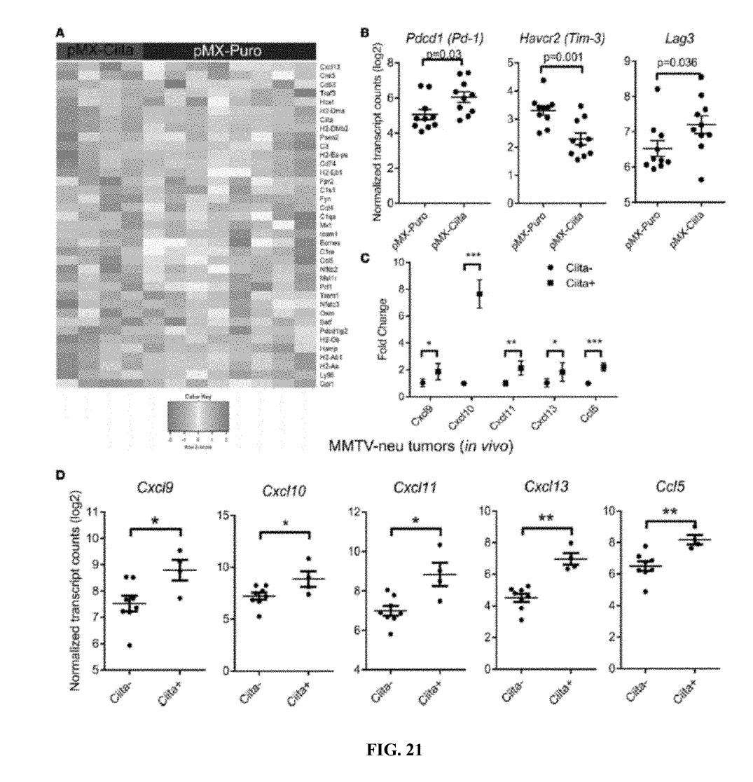

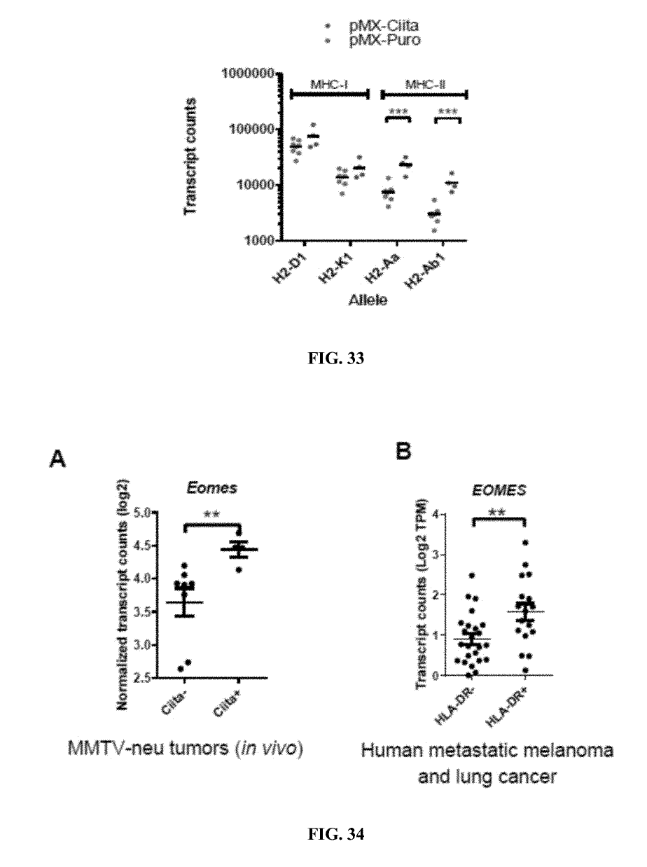

Enforced expression of MHC-II has been shown to result in increased antitumor inflammation, Th1 differentiation, and antigen-specific CD4+ T cells (68-71). Consistent with this, it was found that tumors that did form in the presence of enforced MHC-II had greater fractions of CD4+ T cells (normalized to total TILs), with no change in the regulatory compartment (Foxp3+) (FIG. 20D). CD8+ T cells showed a downward trend when normalized to total TILs; however, this was reflective of the increased percentage of CD4+ T cells, and absolute extent of CD8+ T cell infiltrate was not affected by MHC-II status. Gene expression analysis of refractory (those tumors evading initial immunologic rejection) MHC-II+ and control MMTV-neu tumors showed enhanced expression Ciita-regulated genes (suggesting MHC-II expression was not lost in these tumors) (FIG. 21A and FIG. 33). In addition, both Pdcd1/Pd-1 and Lag3 were more highly expressed in MHC-II+ tumors. Havcr2/Tim-3 was not highly expressed or enriched in MHC-II+ tumors (FIG. 21B). Collectively, these data suggest that MHC-II expression in tumor cells promotes an antitumor immune environment coinciding with recruitment of CD4+ T cells and the eventual engagement of Pd-1 and Lag-3 to suppress antitumor immunity.

MHC-II expression promotes the expression of T cell-recruiting chemokines. The mechanism behind recruitment of T cells to the immune microenvironment of MHC-II+ tumors is unclear. However, known T cell chemoattractant cytokines (e.g., Cxcl13, Ccl5) were elevated in MHC-II+ MMTV-neu tumors (FIG. 21A). To determine if chemokine production was a direct byproduct of Ciita expression, MMTV-neu cells grown ex vivo were evaluated for expression of Cxcl9, Cxcl10, Cxcl11, Cxcl13, and Ccl5. Enhanced expression of these chemokines at the mRNA level were detected, indicating they may be downstream of Ciita transcriptional activation (FIG. 21C). Large-scale ChIP-seq experiments have identified DNA binding of the CIITA/RFX5 complex at the promoter of CXCL9 and CXCL10 (72). Thus, the likely mechanism whereby CIITA expression mediates an enhanced inflammatory environment is via increased chemoattractant cytokines, which promote the recruitment of Th1 and cytotoxic T lymphocytes to the microenvironment, exacerbating inflammatory signals. Expression of each of these ligands was also confirmed in vivo (FIG. 21D). However, eomesodermin (Eomes) expression was elevated as well in these tumors, suggesting the potential for effector T cell exhaustion (FIG. 34A).

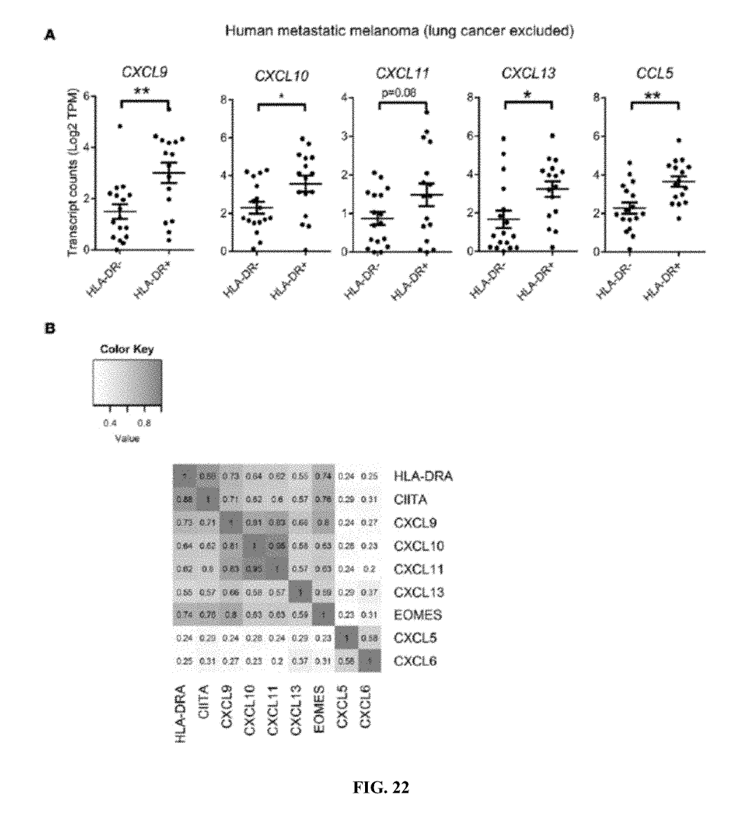

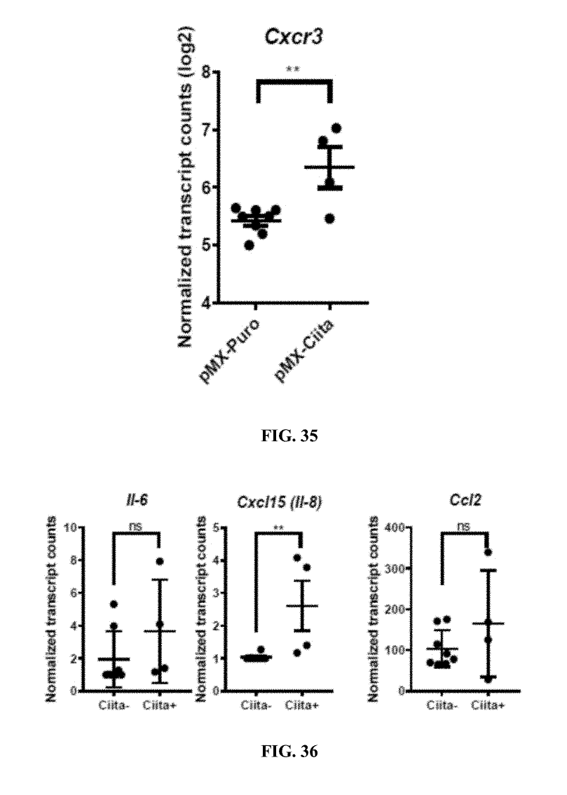

Similar elevation of these markers was present in MHC-II+ melanomas (FIG. 22A and FIG. 34B). A high degree of coexpression was also observed in primary breast cancers, supporting the interdependency of these markers in the MHC-II+ phenotype (FIG. 22B). Cxcl9, Cxcl10, and Cxcl11 (and their human orthologs), which are chiefly produced by tissue-resident and endothelial cells, bind the Cxcr3 receptor on Th1 and cytotoxic T cells to license entry into sites of inflammation (73). Interestingly, increased Cxcr3 expression was also detectable in the tumor microenvironment of Ciita+ MMTV-neu tumors (FIG. 35). In comparison, substantial differential or absolute expression of myeloid chemokines, such as Cc12, Cxcl15 (IL-8), or 11-6 were not observed, although there was a statistically significant increase in 11-8 in Ciita+ tumors (FIG. 36).

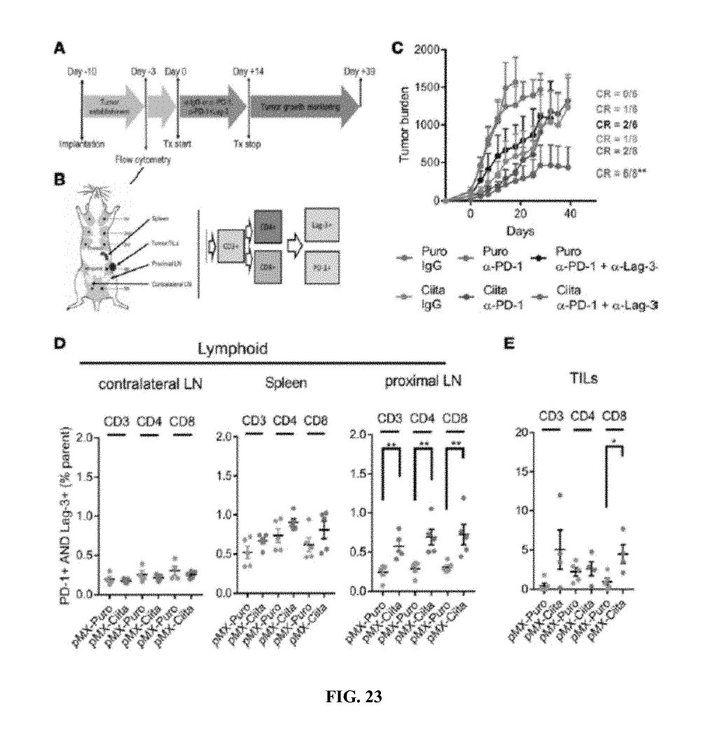

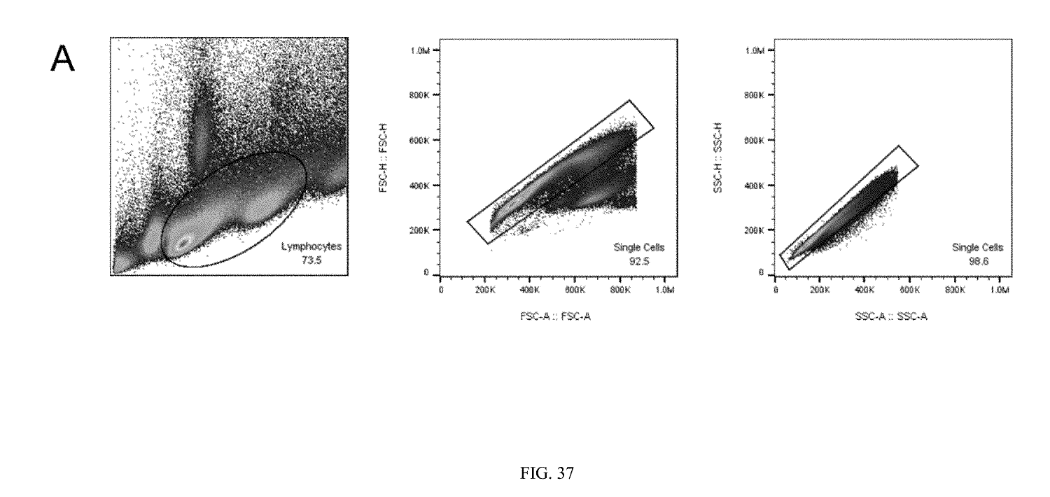

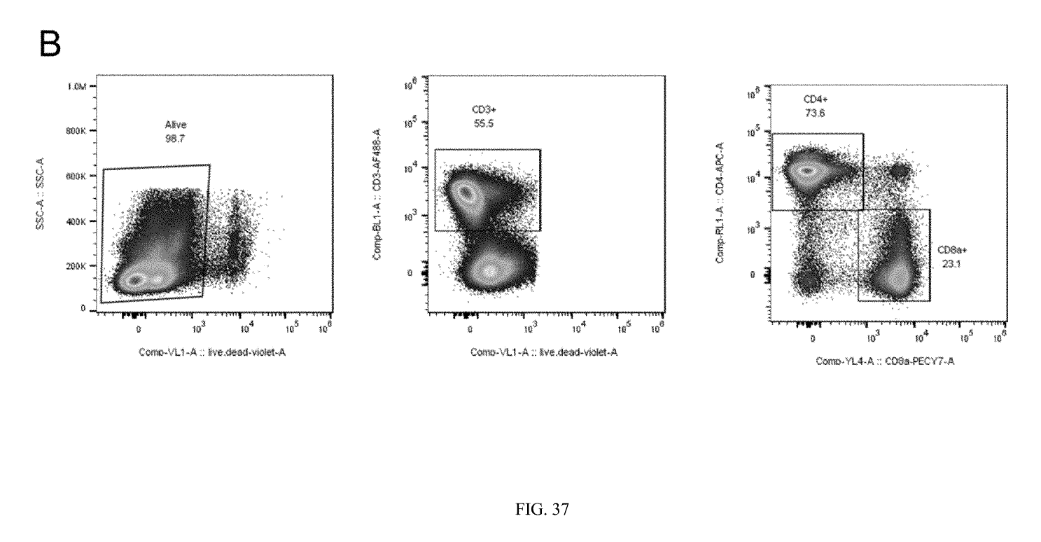

A combination of PD-1 and Lag-3 immune checkpoint inhibitor therapy enhances antitumor immunity in MHC-II⁺ tumors. MMTV-neu tumor cells were utilized transduced to enforce expression of Ciita (or vector control), as above, according to the experimental design shown in FIGS. 23, A and B. Ten days after orthotopic implantation, mice were treated with anti-IgG (control), anti-PD-1, or the combination of anti-PD-1 and anti-Lag-3 for 2 weeks. Only mice with palpable and actively growing tumors at the start of therapy were treated in order to reduce confounders associated with enhanced immunogenicity and rejection observed in untreated Ciita⁺ tumors. A moderate treatment effect was observed in the combination arm in pMX-puro (control) tumors, but a pronounced antitumor effect was observed in the combination arm for Ciita⁺ tumors, with 6 of 8 mice exhibiting complete tumor rejection (P<0.05, χ² test; FIG. 23C). Flow cytometry analysis of lymphoid compartments demonstrated enhanced PD-1⁺/Lag-3⁺ CD4⁺ and CD8⁺ T cells in the proximal lymph node, with a similar trend observed in the spleen but not the contralateral lymph node (FIG. 23D and FIG. 37). A similar effect was observed in tumor-infiltrating CD8 cells (FIG. 23E). Thus, MHC-II positivity on tumor cells can elicit enhanced dependency on T cell checkpoints, including Lag-3, which can be overcome therapeutically.

Alternative MHC-II ligands are upregulated in MHC-II⁺ tumors and promote suppression of effector cell cytotoxicity. MHC-II receptors on lymphocytes may exist with similar functionality to Lag-3. FCRL6 is an immunoreceptor that is structurally related to classical Ig-binding leukocyte Fc receptors but was also shown to be a ligand for MHC-II (74). FCRL6 is an ITIM-bearing Ig superfamily member expressed by cytotoxic NK cells and effector memory CD8⁺ T cells (75, 76). Thus, FCRL6 may function as a novel immune checkpoint to suppress effector cell activity when engaged with MHC-II. The regulation of NK cell cytotoxicity by MHC class I molecules has been well characterized, but evidence also exists that MHC class II expression can protect target cells from NK cell-mediated killing. Enforced expression of HLA-DR by K562 cells, a classic human erythroleukemic MHC-II-negative target cell line, was found to inhibit lysis by freshly isolated human NK cells in vitro (77). Furthermore, transplantation of K562 cells into NOD/SCID mice followed by adoptive transfer of human PBMCs demonstrated that K562 tumors expressing HLA-DR were dramatically protected from elimination by human NK cells in vivo (78).

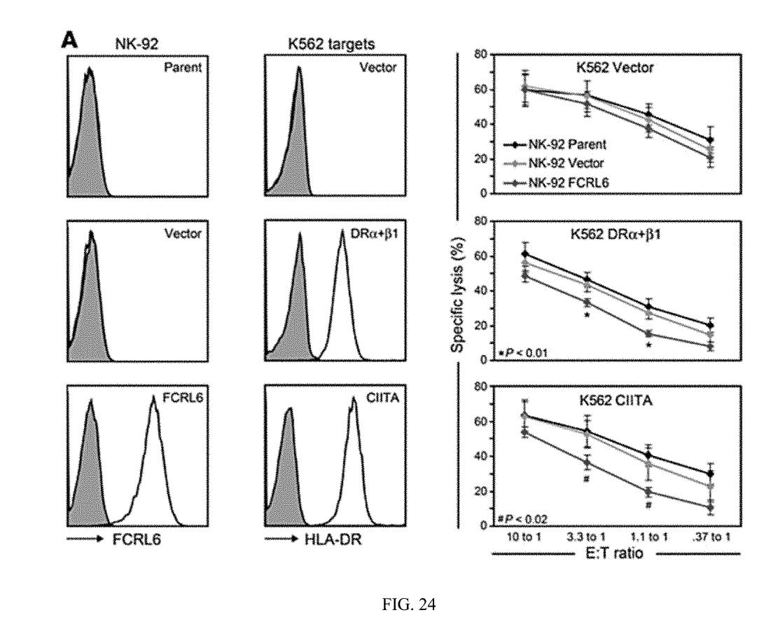

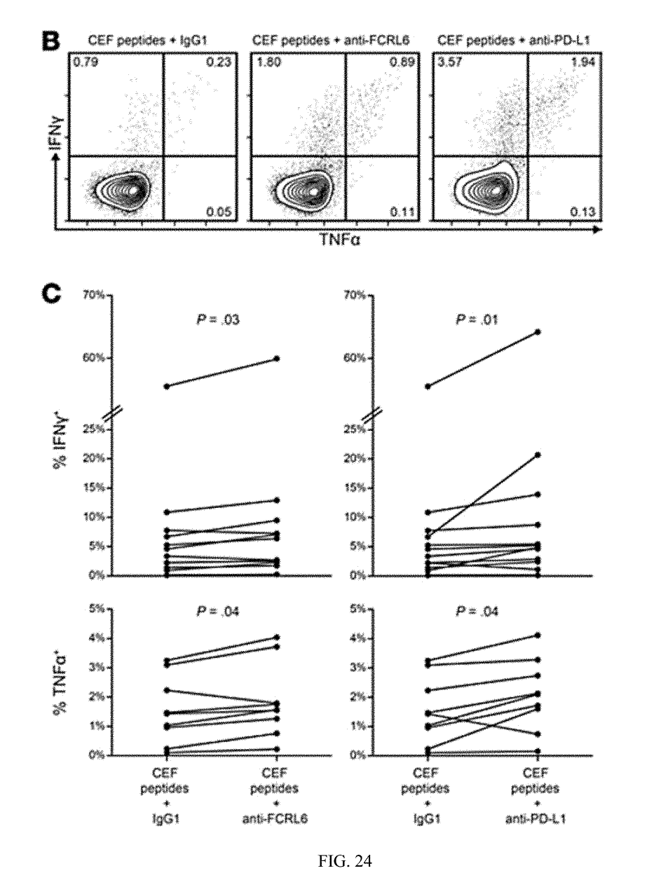

Because FCRL6 is downregulated upon exposure to IL-2 or IL-15 (75), using expanded primary human NK or T cell clones was not a feasible approach for studying its function. In order to determine whether FCRL6 inhibits NK cell-mediated killing of HLA-DR-expressing tumor cells, the NK-92 human cytotoxic NK cell line, which does not endogenously express FCRL6 on its surface, was transduced with either FCRL6 or a vector control (FIG. 24A). In addition, K562 target cells were transduced either with plasmids harboring the HLA-DRα and -DRβ1 subunits or with CITTA to drive endogenous MHC class II expression in these cells. K562 HLA-DRα⁺β1 and CIITA transductants, but not parental K562 cells or control transductants, expressed surface HLA-DR, by flow cytometric analysis (FIG. 24A). K562 cells lack endogenous MHC-I expression and are therefore targeted by NK cell-mediated cytotoxicity. Thus, FCRL6⁺ NK-92 lines were assayed for their ability to kill K562 transductants (MHC-II⁺ and MHC-II⁻) in ⁵¹Cr release experiments. These studies found that FCRL6⁺ NK-92 cells were significantly impaired in their capacity to lyse K562 DRα⁺62 1- or CIITA-expressing targets but not control K562 cells (FIG. 24A). Hence, FCRL6 is an inhibitory NK receptor for HLA-DR. Due to lack of a functional homolog in mice, only human systems are equipped to demonstrate this.

Since mouse FCRL6 differs structurally and functionally from its human relative (79), it is not a viable interspecies translational model for study. To test the hypothesis that the FCRL6/HLA-DR interaction might also inhibit CD8⁺ T cell responses, the effect of FCRL6 blockade during pathogen-specific peptide stimulation in vitro was examined. This analysis employed an anti-FCRL6 mAb (1D8) that is capable of blocking FCRL6 activation in coculture assays and obstructing FCRL6-Fc binding to HLA-DR transductants (74). Blood mononuclear cells from healthy donors were stimulated with pooled antigenic MHC class I-restricted peptides from CMV, EBV, and influenza virus epitopes (CEF peptide pool) in the presence of anti-FCRL6 or anti-PD-L1 (a positive control). Following a 6-day culture period, cells were collected, restimulated with CEF, and assayed for cytokine production by intracellular staining. A significantly higher frequency of CD8⁺ T cells produced IFN-γ and TNF-α when cultured with the FCRL6 or PD-L1 mAbs compared with controls (FIGS. 24, B and C). Thus, these experiments identify the MHC-II receptor FCRL6 as a potential immunotherapeutic target and suppressor of NK and effector T cell activity.

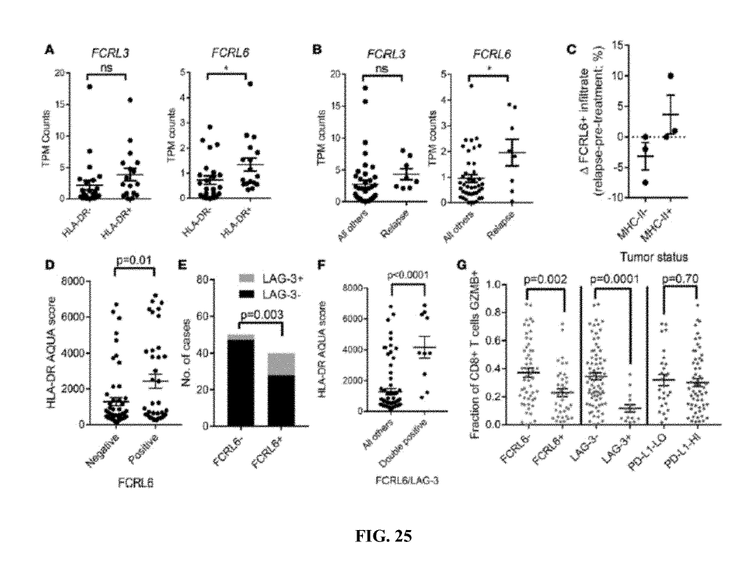

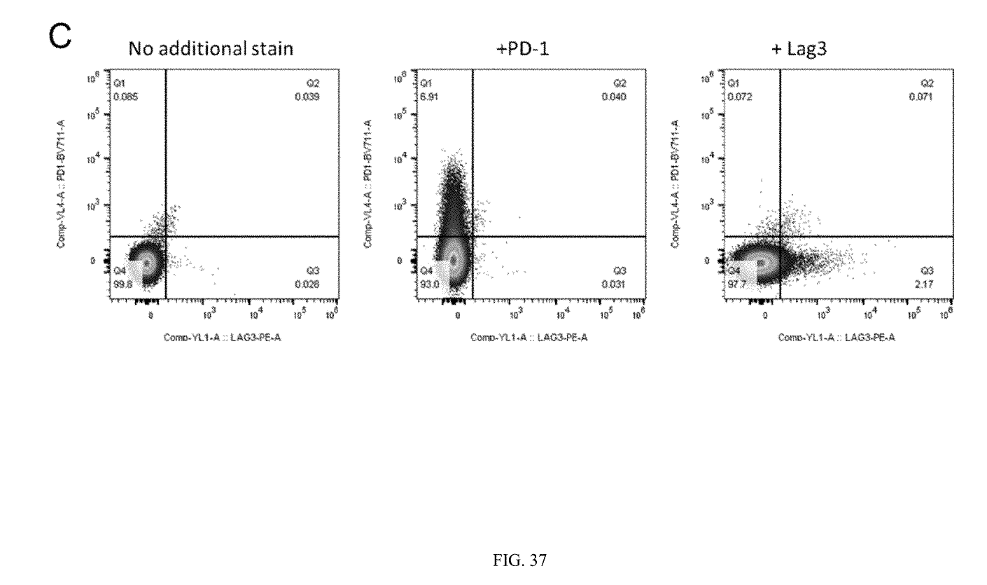

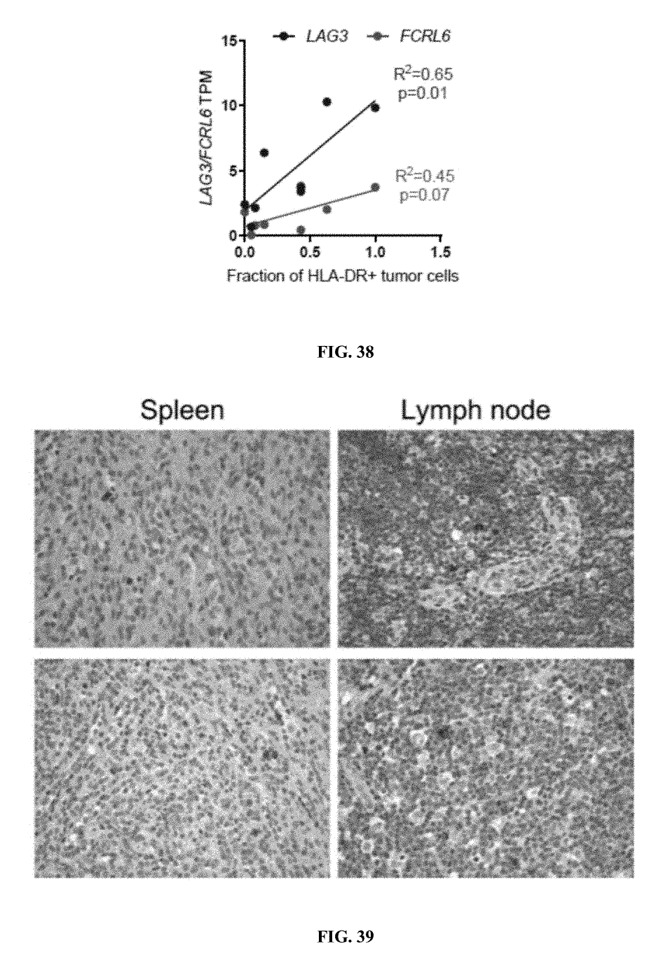

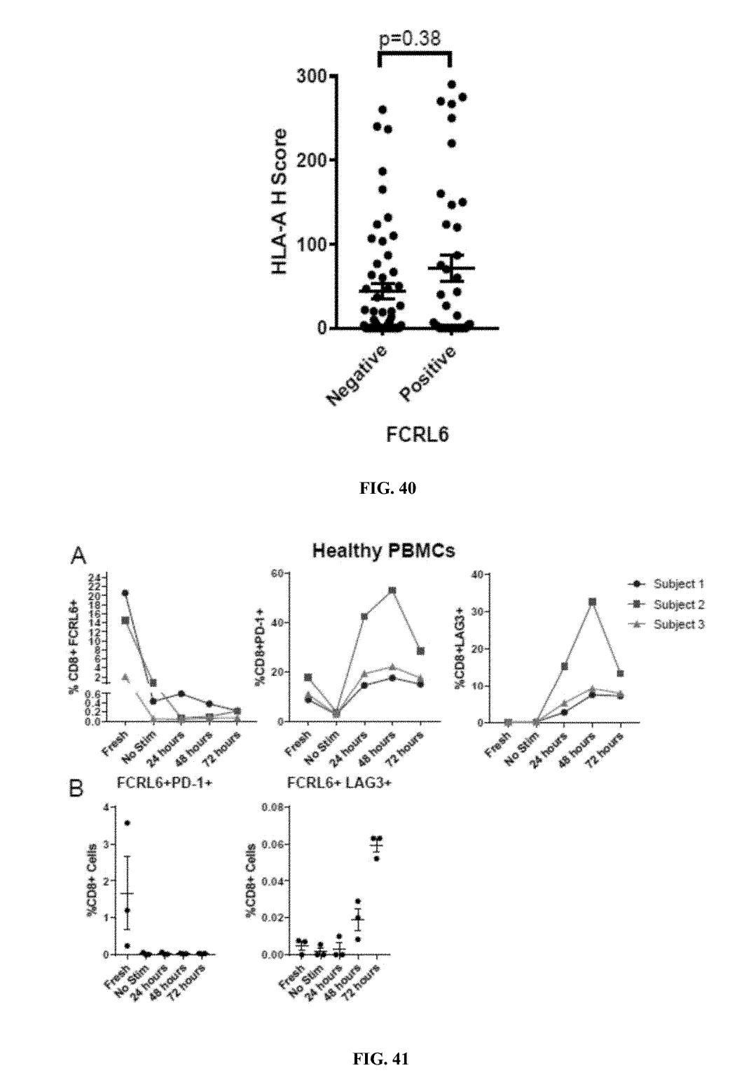

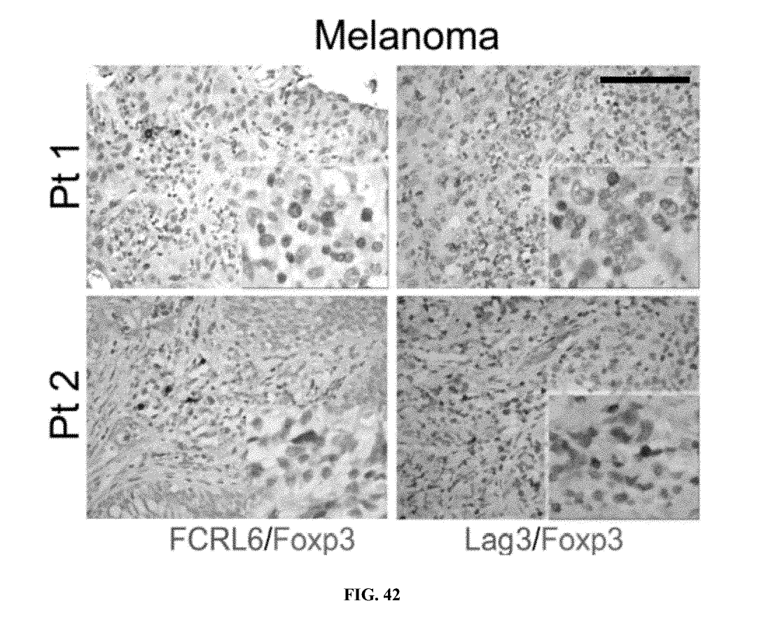

FCRL6, but not its relative FCRL3, was significantly more highly expressed in MHC-II melanoma and lung cancers (FIG. 25A), and the degree of LAG3 and FCRL6 showed a linear relationship with the fraction of HLA-DR⁺ tumor cells (R²=0.65 and 0.45, respectively; FIG. 38). Furthermore, FCRL6 was elevated at relapse after progression on PD-1-targeted therapy both at the mRNA (FIG. 25B) and at the protein level (FIG. 25C and FIG. 39). In TNBC specimens, FCRL6⁺ lymphocytes were associated with tumor HLA-DR status (FIG. 25D) and Lag-3 status (FIG. 25E) but not MHC-I status (HLA-A; FIG. 40). Furthermore, concurrent presence of Lag-3⁺ lymphocytes and FCRL6⁺ lymphocytes was strikingly associated with high tumor-specific HLA-DR expression (FIG. 25F). In healthy human subject PBMCs, activation of T cells by ligation of CD3/CD28 induced PD-1 and LAG3 expression over 24-72 hours but paradoxically and drastically reduced FCRL6 expression (FIG. 41A); however, between 24 and 72 hours, a rare but reproducible population of FCRL6⁺/LAG3⁺ cells was expanded in the CD8 compartment. In contrast, there was no overlap in the expression of PD-1 and LAG3 by T cells, suggesting a potential biological implication of coexpression of FCRL6 and LAG3, which both bind MHC-II (FIG. 41B). Finally, among all breast tumors assessed, the presence of FCRL6 or Lag-3⁺ lymphocytes was associated with fewer cytotoxic CD8⁺ T cells (as defined by granzyme B⁺/CD8⁺ cells), supporting a hypothesis of active immune suppression mediated by these checkpoints (FIG. 25G), particularly in MHC-II⁺ tumors. Importantly, neither Lag-3 nor FCRL6 colocalized to known immunosuppressive T regulatory cells by dual Foxp3/MHC-II receptor IHC analysis in human melanomas (FIG. 42).

### Example 7

TABLE 7

| | n | % | median | sd | units |
|---|---|---|---|---|---|
| Clinical | | | | | |
| Recurrence-free | 94 | 94% | 21.2 | | months |
| survival | | | | | |
| Overall survival | 97 | 97% | 31.2 | | months |
| Histology | | | | | |
| TILs (H&E) | 92 | 92% | 10% | 18.60% | percent positive |
| CD4 (IHC) | 86 | 86% | 3% | 6% | percent positive |
| CD8 (IHC) | 86 | 86% | 3% | 4% | percent positive |
| Lag3 (IHC) | 92 | 92% | 14% samples positive | | percent positive |
| multiplexed IF | | | | | |
| CD4/CD8/GZMB | 83 | 83% | | | |
| (IF/AQUA) | | | | | |
| CD4 total | | | 2.3% | 5% | percent positive |
| CD8 total | | | 4% | 5% | percent positive |
| CD4+GZMB+ total | | | 0.2% | 0.4% | percent positive |
| CD8+GZMB+ total | | | 0.8% | 0.9% | percent positive |
| CD4+GZMB+ | | | 4.5% | 15.0% | percent positive |
| percent of CD4+ | | | | | |
| CD8+GZMB+ | | | 26% | 22.40% | percent positive |
| percent of CD8+ | | | | | |
| PD-L1 (IF/AQUA) | 79 | 79% | | | |
| PD-L1 (tumor) | | | 923 | 326 | AQUA score |
| PD-L1 (stroma) | | | 801 | 303 | AQUA score |
| PD-L1 (total) | | | 1725 | 601 | AQUA score |

Table 7 shows the biomarkers in a series of 100 residual TNBCs after neo-adjuvant chemotherapy

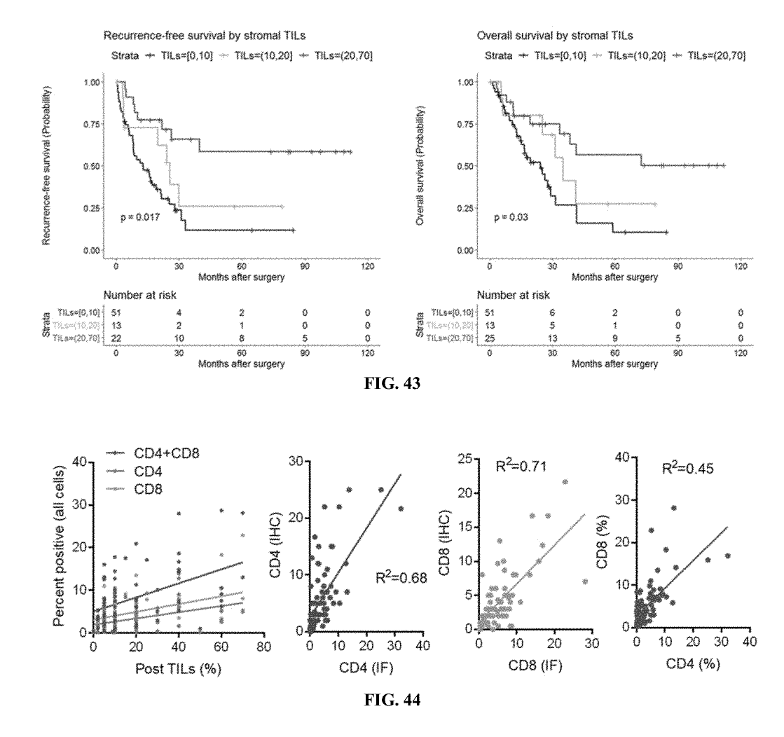

FIG. 43 shows the association of sTILs with outcome after surgery. Patients with tumors that are heavily infiltrated present with the best survival. (FIGS. 43A and B).

There are correlations between CD4+ and CD8+ T-cells which demonstrate that tumor infiltrating lymphocytes are likely composed of CD4+ and CD8+ T-cells. FIGS. 44(A-D) shows that a significant percentage of sTIL cells were positive for CD4 or CD8.

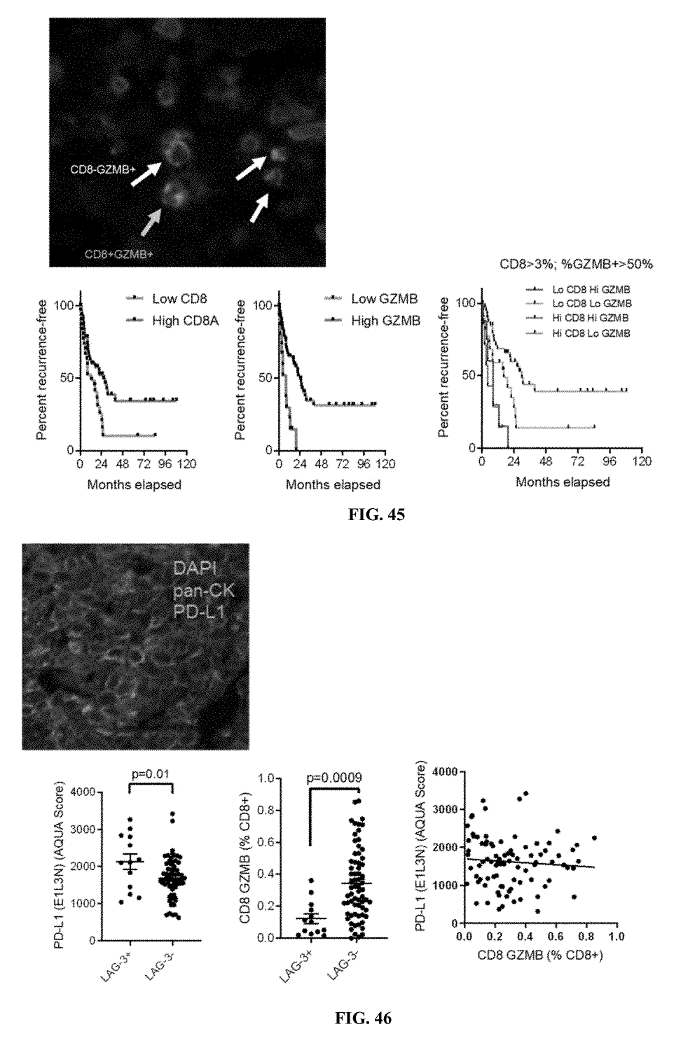

FIG. 45 shows the correlation between the association of T-cell composition with outcome. Dual positive cells (CD4+GZMB+ and CD8+GZMB+) were identified and quantified by AQUA and tested for association with clinical parameters. CD8+ and CD4+ cells were also enumerated without respect to GZMB composition as an additional parameter.

FIG. 46 shows checkpoint expression. The checkpoint LAG3 and PD-1 ligand PD-L1 were quantified by IHC and multiplexed IF (AQUA), respectively. PD-L1 was associated with presence of LAG3+ TILs, while LAG3 presence was also associated with GZMB+CD8+ T cells. However, no direct association was observed between GZMB+CD8+ T cells and PD-L1.

TABLE 8

Cox proportional hazards

| | RFS (p-value) | OS (p-value) |
|---|---|---|
| sTILs | 0.0001 | 0.002 |
| Total CD8 (IHC) | 0.005 | 0.007 |
| Total CD4 (IHC) | 0.0006 | 0.003 |
| Total CD8 (IF) | 0.15 | 0.004 |
| Total CD4 (IF) | 0.05 | 0.005 |
| Total GZMB+CD8 (IF) | 0.04 | 0.22 |
| Total GZMB+CD4 (IF) | 0.04 | 0.02 |
| % GZMB+ of CD8 | 0.004 | 0.0004 |
| % GZMB+ of CD4 | 0.05 | 0.09 |
| PD-L1 (E1L3N) total (IF) | 0.06 | 0.94 |
| PD-L1 (E1L3N) stroma (IF) | 0.04 | 0.67 |
| PD-L1 (E1L3N) tumor (IF) | 0.13 | 0.84 |

Univariate analysis for parameters collected by IHC or IF and association (Cox proportional hazards) with RFS and OS. Italicized p-values represent a beneficial association, bolded p-values represent a detrimental association. The paradoxical finding that GZMB+ CD8+ T cells are a negative prognostic factor for long term outcome in NAC-treated TNBC is an intriguing result. Table 8.

TABLE 9

| | coef | exp(coef) | se(coef) | z | p |
|---|---|---|---|---|---|
| Post-treatment TILs | 0.0068 | 1.006823 | 0.013362 | 0.509 | 0.61085 |
| CD4 (IHC) | −0.23006 | 0.794484 | 0.094369 | −2.438 | [symbol] |
| CD8 (IHC) | 0.174397 | 1.190529 | 0.112363 | 1.552 | 0.12064 |
| CD8_GZMB_CD8 | 2.247598 | 9.464974 | 0.86071 | 2.611 | 0.00902 |
| PD-L1_POST_STROMA_MEAN | 0.001278 | 1.001278 | 0.000635 | 2.013 | 0.0441 |

Likelihood ratio test p = 3.52e−05

All significant and marginally significant parameters from univariate analyses were used to construct a multivariate model to test independence of variables in predicting RFS. Post-treatment TILs and CD8+ T cells lost significance in the presence of PD-L1, GZMB+CD8+ cells, and CD4+ T cells. Italicized p-values represent a beneficial association, bolded p-values represent a detrimental association. Table 9. In a multivariate analysis, total CD4 cells were a positive predictor of RFS on post-NAC TNBC, while CD8+GZMB+ cells and stromal PD-L1 were negative predictors.

### Example 8

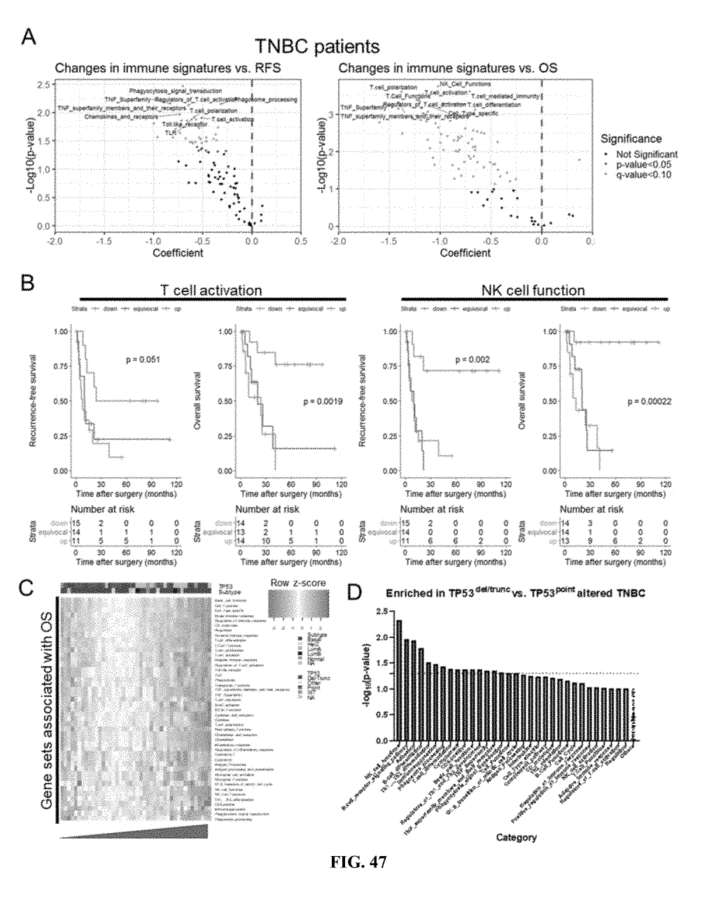

TP53 mutations have distinct chemokine and immune expression signatures. FIG. 47 demonstrates using chemokine expression derived from gene expression data using nanostring gene expression profiling. FIG. 47 further shows that certain TP53 mutations, as detected via directed TP53 sequencing have different profiles of chemokines and immune expression signatures. (FIG. 47C).

### Example 9

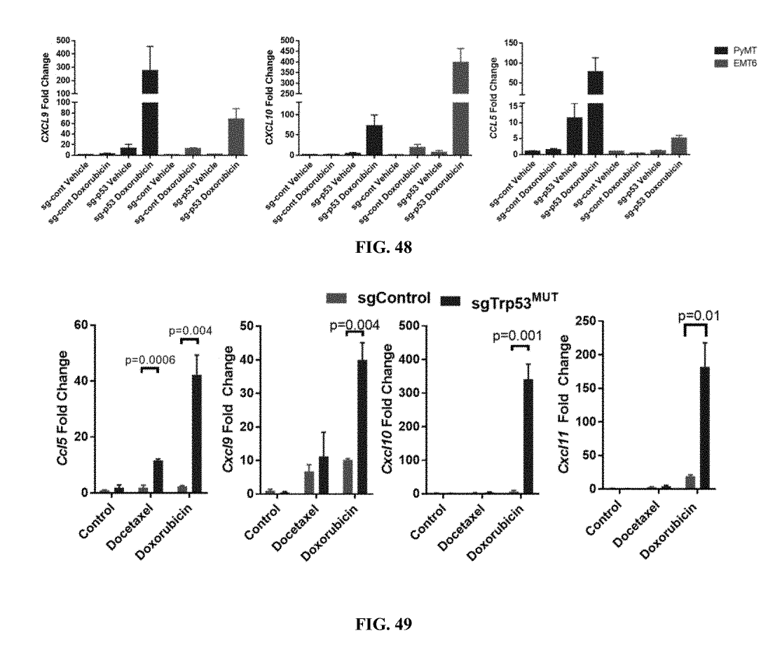

TP53 mutant mouse breast cancer cells lines induce cytokine expression hollowing doxorubicin treatment. FIG. 48.

Doxorubicin induces T cell recruiting chemokines in p53 altered MMTV-Neu cells. FIG. 49.

### Example 10

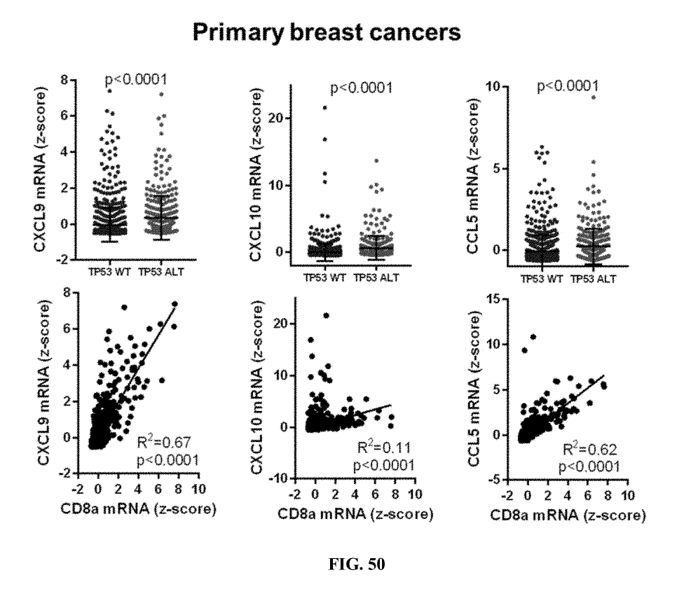

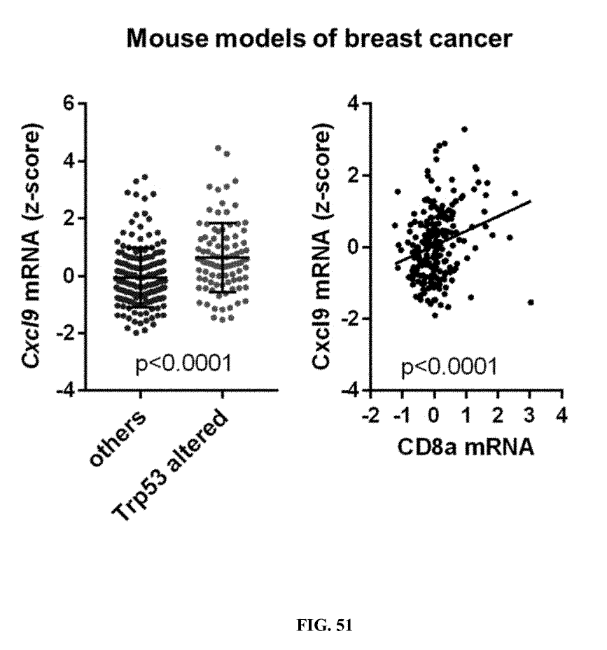

Mined data from the Cancer Genome Atlas (TCGA) showing higher expression of chemokines in human breast tumors with TP53 gene mutations. FIG. 50-51. The expression of these chemokines also is associated with CD8A, a gene expression marker of T cells, in the same tumors. Below that is data from >200 genetic mouse breast cancer models showing TP53-altered mouse models express greater T cell chemokines, and again, in mouse models as with human breast tumors, the expression of these chemokines are associated with T cell markers like CD8A gene expression. FIG. 50-51.

All publications, patents, and patent applications mentioned in this specification are herein incorporated by reference to the same extent as if each individual publication, patent, or patent application was specifically and individually indicated to be incorporated by reference, including the references set forth in the following list:
1. Topalian, S. L., et al. Safety, activity, and immune correlates of anti-PD-1 antibody in cancer. The New England journal of medicine 366, 2443-2454 (2012).
2. Hamid, O., et al. Safety and Tumor Responses with Lambrolizumab (Anti-PD-1) in Melanoma. The New England journal of medicine 369, 134-144 (2013).
3. Herbst, R. S., et al. Predictive correlates of response to the anti-PD-L1 antibody NIPDL3280A in cancer patients. Nature 515, 563-567 (2014).
4. Robert, C., et al. Nivolumab in Previously Untreated Melanoma without BRAF Mutation. The New England journal of medicine (2014).
5. Robert, C., et al. Anti-programmed-death-receptor-1 treatment with pembrolizumab in ipilimumab-refractory advanced melanoma: a randomised dose-comparison cohort of a phase 1 trial. Lancet (2014).
6. Robert, C., et al. Pembrolizumab versus Ipilimumab in Advanced Melanoma. The New England journal of medicine (2015).
7. Rizvi, N. A., et al. Activity and safety of nivolumab, an anti-PD-1 immune checkpoint inhibitor, for patients with advanced, refractory squamous non-small-cell lung cancer (CheckMate 063): a phase 2, single-arm trial. The lancet oncology 16, 257-265 (2015).
8. Garon, E. B., et al. Pembrolizumab for the Treatment of Non-Small-Cell Lung Cancer. The New England journal of medicine (2015).
9. Gettinger, S. N., et al. Overall Survival and Long-Term Safety of Nivolumab (Anti-Programmed Death 1 Antibody, BMS-936558, ONO-4538) in Patients With Previously Treated Advanced Non-Small-Cell Lung Cancer. Journal of clinical oncology: official journal of the American Society of Clinical Oncology (2015).
10. Motzer, R. J., et al. Nivolumab for Metastatic Renal Cell Carcinoma: Results of a Randomized Phase II Trial. Journal of clinical oncology: official journal of the American Society of Clinical Oncology (2014).
11. Powles, T., et al. MPDL3280A (anti-PD-L1) treatment leads to clinical activity in metastatic bladder cancer. Nature 515, 558-562 (2014).
12. Ansell, S. M., et al. PD-1 Blockade with Nivolumab in Relapsed or Refractory Hodgkin's Lymphoma. The New England journal of medicine (2014).
13. Nanda, R., Chow L Q, Dees E C, Berger R, Gupta S, et al. A phase Ib study of pembrolizumab (MK-3475) in patients with advanced triple-negative breast cancer. in San Antonio Breast Cancer Symposium (2014).
14. Seiwert, T. Y., Burtness B, Weiss J, Gluck I, Eder J P, et al. A phase Ib study of MK-3475 in patients with human papillomavirus (HPV)-associated and non-HPV-associated head and neck (H/N) cancer. Journal of clinical oncology: official journal of the American Society of Clinical Oncology 32, 6011 (2014).
15. Brahmer, J. R., et al. Phase I study of single-agent anti-programmed death-1 (MDX-1106) in refractory solid tumors: safety, clinical activity, pharmacodynamics, and immunologic correlates. Journal of clinical oncology: official journal of the American Society of Clinical Oncology 28, 3167-3175 (2010).
16. Snyder, A., et al. Genetic Basis for Clinical Response to CTLA-4 Blockade in Melanoma. The New England journal of medicine (2014).
17. Yadav, M., et al. Predicting immunogenic tumour mutations by combining mass spectrometry and exome sequencing. Nature 515, 572-576 (2014).
18. Tumeh, P. C., et al. PD-1 blockade induces responses by inhibiting adaptive immune resistance. Nature 515, 568-571 (2014).
19. Spranger, S., Bao, R. & Gajewski, T. F. Melanoma-intrinsic beta-catenin signalling prevents anti-tumour immunity. Nature 523, 231-235 (2015).
20. Mellman, I., Coukos, G. & Dranoff, G. Cancer immunotherapy comes of age. Nature 480, 480-489 (2011).
21. Mikucki, M. E., et al. Non-redundant requirement for CXCR3 signalling during tumoricidal T-cell trafficking across tumour vascular checkpoints. Nat Commun 6, 7458 (2015).
22. Garrido, F., Cabrera, T. & Aptsiauri, N. “Hard” and “soft” lesions underlying the HLA class I alterations in cancer cells: implications for immunotherapy. Int J Cancer 127, 249-256 (2010).
23. Warabi, M., Kitagawa, M. & Hirokawa, K. Loss of MEW class II expression is associated with a decrease of tumor-infiltrating T cells and an increase of metastatic potential of colorectal cancer: immunohistological and histopathological analyses as compared with normal colonic mucosa and adenomas. Pathol Res Pract 196, 807-815 (2000).
24. Bernsen, M. R., et al. On the biological relevance of MEW class II and B7 expression by tumour cells in melanoma metastases. Br J Cancer 88, 424-431 (2003).
25. Oldford, S. A., et al. Tumor cell expression of HLA-DM associates with a Th1 profile and predicts improved survival in breast carcinoma patients. Int Immunol 18, 1591-1602 (2006).
26. Degenhardt, Y., et al. Distinct MHC gene expression patterns during progression of melanoma. Genes Chromosomes Cancer 49, 144-154 (2010).
27. Pollack, M. S., Heagney, S. D., Livingston, P. O. & Fogh, J. HLA-A, B, C and DR alloantigen expression on forty-six cultured human tumor cell lines. Journal of the National Cancer Institute 66, 1003-1012 (1981).
28. Barbieri, G., Rimini, E. & Costa, M. A. Effects of human leukocyte antigen (HLA)-DR engagement on melanoma cells. International journal of oncology 38, 1589-1595 (2011).
29. Colloby, P. S., West, K. P. & Fletcher, A. Is poor prognosis really related to HLA-DR expression by malignant melanoma cells? Histopathology 20, 411-416 (1992).
30. Chornoguz, O., Gapeev, A., O'Neill, M. C. & Ostrand-Rosenberg, S. Major histocompatibility complex class II+ invariant chain negative breast cancer cells present unique peptides that activate tumor-specific T cells from breast cancer patients. Mol Cell Proteomics 11, 1457-1467 (2012).
31. Londei, M., Lamb, J. R., Bottazzo, G. F. & Feldmann, M. Epithelial cells expressing aberrant MHC class II determinants can present antigen to cloned human T cells. Nature 312, 639-641 (1984).
32. Meazza, R., Comes, A., Orengo, A. M., Ferrini, S. & Accolla, R. S. Tumor rejection by gene transfer of the MHC class II transactivator in murine mammary adenocarcinoma cells. Eur J Immunol 33, 1183-1192 (2003).
33. Rizvi, N. A., et al. Cancer immunology. Mutational landscape determines sensitivity to PD-1 blockade in non-small cell lung cancer. Science 348, 124-128 (2015).
34. Wellbrock, C., et al. STATS contributes to interferon resistance of melanoma cells. Curr Biol 15, 1629-1639 (2005).
35. Lee, Y. S., Kim, S. H., Cho, J. A. & Kim, C. W. Introduction of the CIITA gene into tumor cells produces exosomes with enhanced anti-tumor effects. Exp Mol Med 43, 281-290 (2011).
36. Joseph, R. W., et al. Correlation of NRAS mutations with clinical response to high-dose IL-2 in patients with advanced melanoma. J Immunother 35, 66-72 (2012).
37. Johnson, D. B., et al. Impact of NRAS Mutations for Patients with Advanced Melanoma Treated with Immune Therapies. Cancer immunology research 3, 288-295 (2015).
38. Loor, F. & Kindred, B. Differentiation of T-cell precursors in nude mice demonstrated by immunofluorescence of T-cell membrane markers. J Exp Med 138, 1044-1055 (1973).
39. Rodriguez, T., et al. Patterns of constitutive and IFN-gamma inducible expression of HLA class II molecules in human melanoma cell lines. Immunogenetics 59, 123-133 (2007).
40. Wolchok, J. D., et al. Nivolumab plus Ipilimumab in Advanced Melanoma. The New England journal of medicine (2013).
41. Postow, M. A., et al. Nivolumab and Ipilimumab versus Ipilimumab in Untreated Melanoma. The New England journal of medicine (2015).
42. Larkin, J., et al. Combined Nivolumab and Ipilimumab or Monotherapy in Untreated Melanoma. N Engl J Med 373, 23-34 (2015).
43. Benjamini, Y. & Hochberg, Y. Controlling the False Discovery Rate: A Practical and Powerful Approach to Multiple Testing. Journal of the Royal Statistical Society. Series B (Methodological) 57, 289-300 (1995).
44. Efron, B. & Tibshirani, R. On testing the significance of sets of genes. Annals of Applied Statistics 1, 107-129 (2007).
45. Balko, J. M., et al. Profiling of residual breast cancers after neoadjuvant chemotherapy identifies DUSP4 deficiency as a mechanism of drug resistance. Nat Med 18, 1052-1059 (2012).
46. Balko, J. M., et al. Activation of MAPK pathways due to DUSP4 loss promotes cancer stem cell-like phenotypes in basal-like breast cancer. Cancer Res (2013).
47. Barretina, J., et al. The Cancer Cell Line Encyclopedia enables predictive modelling of anticancer drug sensitivity. Nature 483, 603-607 (2012).
48. Balar A V, Weber J S. PD-1 and PD-L1 antibodies in cancer: current status and future directions. Cancer Immunol Immunother. 2017; 66(5):551-564.
49. Shin D S, et al. Primary resistance to PD-1 blockade mediated by JAK1/2 mutations. Cancer Discov. 2017; 7(2):188-201.
50. Zaretsky J M, et al. Mutations associated with acquired resistance to PD-1 blockade in melanoma. N Engl J Med. 2016; 375(9):819-829.
51. Koyama S, et al. Adaptive resistance to therapeutic PD-1 blockade is associated with upregulation of alternative immune checkpoints. Nat Commun. 2016; 7:10501.
52. Lu Y C, Robbins P F. Cancer immunotherapy targeting neoantigens. Semin Immunol. 2016; 28(1):22-27.
53. Rizvi N A, et al. Cancer immunology. Mutational landscape determines sensitivity to PD-1 blockade in non-small cell lung cancer. Science. 2015; 348(6230):124-128.
54. Schumacher T N, Schreiber R D. Neoantigens in cancer immunotherapy. Science. 2015; 348(6230):69-74.
55. Snyder A, et al. Genetic basis for clinical response to CTLA-4 blockade in melanoma. N Engl J Med. 2014; 371(23):2189-2199.
56. Van Allen E M, et al. Genomic correlates of response to CTLA-4 blockade in metastatic melanoma. Science. 2015; 350(6257):207-211.
57. Zou W, Wolchok J D, Chen L. PD-L1 (B7-H1) and PD-1 pathway blockade for cancer therapy: mechanisms, response biomarkers, and combinations. Sci Transl Med. 2016; 8(328):328rv4.
58. Hugo W, et al. Genomic and transcriptomic features of response to anti-PD-1 therapy in metastatic melanoma. Cell 2016; 165(1):35-44.
59. Johnson D B, et al. Melanoma-specific MHC-II expression represents a tumour-autonomous phenotype and predicts response to anti-PD-1/PD-L1 therapy. Nat Commun. 2016; 7:10582.
60. Roemer M G M, et al. Major histocompatibility complex class II and programmed death ligand 1 expression predict outcome after programmed death 1 blockade in classic Hodgkin lymphoma. J Clin Oncol. 2018; 36(10):942-950.
61. Rodig S J, et al. MEW proteins confer differential sensitivity to CTLA-4 and PD-1 blockade in untreated metastatic melanoma. Sci Transl Med. 2018; 10(450):eaar3342.
62. Johnson D B, et al. Quantitative spatial profiling of PD-1/PD-L1 interaction and HLA-DR/IDO-1 predicts improved outcomes of anti-PD-1 therapies in metastatic melanoma. Clin Cancer Res. 2018; 24(21):5250-5260.
63. Haastert B, Mellanby R J, Anderton S M, O'Connor R A. T cells at the site of autoimmune inflammation show increased potential for trogocytosis. PLoS One. 2013; 8(12):e81404.
64. Baixeras E, et al. Characterization of the lymphocyte activation gene 3-encoded protein. A new ligand for human leukocyteantigen class II antigens. J Exp Med. 1992; 176(2):327-337.
65. Huard B, Tournier M, Hercend T, Triebel F, Faure F. Lymphocyte-activation gene 3/major histocompatibility complex class II interaction modulates the antigenic response of CD4+ T lymphocytes. Eur J Immunol. 1994; 24(12):3216-3221.
66. Ashizawa T, et al. Antitumor effect of programmed death-1 (PD-1) blockade in humanized the NOG-MHC double knockout mouse. Clin Cancer Res. 2017; 23(1):149-158.
67. Loi S, et al. RAS/MAPK activation is associated with reduced tumor-infiltrating lymphocytes in triple-negative breast cancer: therapeutic cooperation between MEK and PD-1/PD-L1 immune checkpoint inhibitors. Clin Cancer Res. 2016; 22(6):1499-1509.
68. Lee Y S, Kim S H, Cho J A, Kim C W. Introduction of the CIITA gene into tumor cells produces exosomes with enhancedanti-tumor effects. Exp Mol Med. 2011; 43(5):281-290.
69. Meazza R, Comes A, Orengo A M, Ferrini S, Accolla R S. Tumor rejection by gene transfer of the MHC class II transactivator in murine mammary adenocarcinoma cells. Eur J Immunol. 2003; 33(5):1183-1192.
70. Mortara L, et al. CIITA-induced MHC class II expression in mammary adenocarcinoma leads to a Th1 polarization of the tumor microenvironment, tumor rejection, and specific antitumor memory. Clin Cancer Res. 2006; 12(11 Pt 1):3435-3443.
71. Yan H, et al. CD4+ T cell-mediated cytotoxicity eliminates primary tumor cells in metastatic melanoma through high MHC class II expression and can be enhanced by inhibitory receptor blockade. Tumour Biol. 2016; 37(12):15949-15958.
72. Wong D, et al. Genomic mapping of the MHC transactivator CIITA using an integrated ChIP-seq and genetical genomics approach. Genome Biol. 2014; 15(10):494.
73. Groom J R, Luster A D. CXCR3 in T cell function. Exp Cell Res. 2011; 317(5):620-631.
74. Schreeder D M, Cannon J P, Wu J, Li R, Shakhmatov M A, Davis R S. Cutting edge: FcR-like 6 is an MHC class II receptor. J Immunol. 2010; 185(1):23-27.
75. Wilson T J, Presti R M, Tassi I, Overton E T, Cella M, Colonna M. FcRL6, a new ITIM-bearing receptor on cytolytic cells, is broadly expressed by lymphocytes following HIV-1 infection. Blood. 2007; 109(9):3786-3793.
76. Schreeder D M, Pan J, Li F J, Vivier E, Davis R S. FCRL6 distinguishes mature cytotoxic lymphocytes and is upregulated in patients with B-cell chronic lymphocytic leukemia. Eur J Immunol. 2008; 38(11):3159-3166.
77. Jiang Y Z, et al. Interaction of natural killer cells with MHC class II: reversal of HLA-DR1-mediated protection of K562 transfectant from natural killer cell-mediated cytolysis by brefeldin-A. Immunology. 1996; 87(3):481-486.
78. Weichold F F, et al. Regulation of a graft-versus-leukemia effect by major histocompatibility complex class II molecules on leukemia cells: HLA-DR1 expression renders K562 cell tumors resistant to adoptively transferred lymphocytes in severe combined immunodeficiency mice/nonobese diabetic mice. Blood. 1997; 90(11):4553-4558.
79. Davis R S. Fc receptor-like molecules. Annu Rev Immunol. 2007; 25:525-560.
80. Thompson J A, Dissanayake S K, Ksander B R, Knutson K L, Disis M L, Ostrand-Rosenberg S. Tumor cells transduced with the MHC class II Transactivator and CD80 activate tumor-specific CD4+ T cells whether or not they are silenced for invariant chain. Cancer Res. 2006; 66(2):1147-1154.
81. Haabeth O A, et al. How do CD4(+) T cells detect and eliminate tumor cells that either lack or express MHC class II molecules? Front Immunol. 2014; 5:174.
82. Bou Nasser Eddine F, Forlani G, Lombardo L, Tedeschi A, Tosi G, Accolla R S. CIITA-driven MHC class II expressing tumor cells can efficiently prime naive CD4+ TH cells in vivo and vaccinate the host against parental MHC-II-negative tumor cells. Oncoimmunology. 2017; 6(1):e1261777.
83. Guerry D, et al. HLA-DR histocompatibility leukocyte antigens permit cultured human melanoma cells from early but not advanced disease to stimulate autologous lymphocytes. J Clin Invest. 1984; 73(1):267-271.
84. Matsuzaki J, et al. Nonclassical antigen-processing pathways are required for MEW class II-restricted direct tumor recognition by NY-ESO-1-specific CD4(+) T cells. Cancer Immunol Res. 2014; 2(4):341-350.
85. Wang D Y, et al. Clinical features of acquired resistance to anti-PD-1 therapy in advanced melanoma. Cancer Immunol Res. 2017; 5(5):357-362.
86. Ascierto P A, et al. Initial efficacy of anti-lymphocyte activation gene-3 (anti-LAG-3; BMS-986016) in combination with nivolumab (nivo) in pts with melanoma (MEL) previously treated with anti-PD-1/PD-L1 therapy. J Clin Oncol. 2017; 35 (Suppl 15):9520.
87. Balko J M, et al. Molecular profiling of the residual disease of triple-negative breast cancers after neoadjuvant chemotherapy identifies actionable therapeutic targets. Cancer Discov. 2014; 4(2):232-245.
88. Balko J M, et al. Triple-negative breast cancers with amplification of JAK2 at the 9p24 locus demonstrate JAK2-specific dependence. Sci Transl Med. 2016; 8(334):334ra53.
89. Schreeder D M, Pan J, Li F J, Vivier E, Davis R S. FCRL6 distinguishes mature cytotoxic lymphocytes and is upregulated in patients with B-cell chronic lymphocytic leukemia. Eur J Immunol. 2008; 38(11):3159-3166.
90. Cerami E, et al. The cBio cancer genomics portal: an open platform for exploring multidimensional cancer genomics data. Cancer Discov. 2012; 2(5):401-404.
91. Cancer Genome Atlas Network. Comprehensive molecular portraits of human breast tumours. Nature. 2012; 490(7418):61-70.
92. Zufferey R, et al. Self-inactivating lentivirus vector for safe and efficient in vivo gene delivery. J Virol. 1998; 72(12):9873-9880.
93. Ehrhardt G R, Davis R S, Hsu J T, Leu C M, Ehrhardt A, Cooper M D. The inhibitory potential of Fc receptor homolog 4 on memory B cells. Proc Natl Acad Sci USA. 2003; 100(23):13489-13494.
94. Fienberg H G, Simonds E F, Fantl W J, Nolan G P, Bodenmiller B. A platinum-based covalent viability reagent for single-cell mass cytometry. Cytometry A. 2012; 81(6):467-475.
95. Kotecha N, Krutzik P O, Irish J M. Web-based analysis and publication of flow cytometry experiments. Curr Protoc Cytom. 2010;Chapter 10:Unit10.17.
96. Amir el-AD, et al. viSNE enables visualization of high dimensional single-cell data and reveals phenotypic heterogeneity of leukemia. Nat Biotechnol. 2013; 31(6):545-552.
97. Bruggner R V, Bodenmiller B, Dill D L, Tibshirani R J, Nolan G P. Automated identification of stratifying signatures in cellular subpopulations. Proc Natl Acad Sci USA. 2014; 111(26):E2770-E2777.
98. Andrew S. Babraham Institute. FastQC: a quality control tool for high throughput sequence data. https://www.bioinformatics. babraham.ac.uk/projects/fastqc/. Accessed Nov. 15, 2018.
99. Aken B L, et al. The Ensembl gene annotation system. Database (Oxford). 2016; 2016:baw093.
100. Dobin A, et al. STAR: ultrafast universal RNA-seq aligner. Bioinformatics. 2013; 29(1):15-21.
101. Dobin A, Gingeras T R. Mapping RNA-seq reads with STAR. Curr Protoc Bioinformatics. 2015; 51:11.14.1-11.1419.
102. Wang L, Wang S, Li W. RSeQC: quality control of RNA-seq experiments. Bioinformatics. 2012; 28(16):2184-2185.
103. Li B, Dewey C N. RSEM: accurate transcript quantification from RNA-Seq data with or without a reference genome. BMC Bioinformatics. 2011; 12:323.
104. Whiteside T L. Measurement of cytotoxic activity of NK/LAK cells. Curr Protoc Immunol. 2001; Chapter 7:Unit 7.18.

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A method of examining a tumor sample from a subject, comprising:
(a) obtaining a tumor sample from a subject;
(b) detecting cell membrane expression of a MHC molecule on a cell from the tumor sample; and
(c) conducting one or more of steps (i)-(iv), including
(i) determining the presence of tumor-infiltrating T cells in the tumor sample;
(ii) determining the presence of tumor-infiltrating lymphocytes in the tumor sample;
(iii) detecting chemokine expression in the tumor sample; and
(iv) detecting TP53 mutations in the tumor sample.

2. The method of claim 1, wherein the MHC molecule is selected from FILA-A, E1LA-B, HLA-C, FILA-DO, HLA-DM, HLA-DR, HLA-DP, HLA-DQ, and HLA-DX.

3. The method of claim 1, wherein the T cells are selected from CD4+ and CD8+ T cells.

4. The method of claim 1, wherein the chemokines are selected from the group consisting of CCL5, CXCL9, CXCL10, and CXCL11.

5. The method of claim 1, wherein the cell membrane expression of MHC molecule is measured using at least one method selected from the group consisting of immunohistochemistry, immunofluorescence, flow cytometry, mass-spectroscopy, RNA sequencing, RNA in situ hybridization, polymerase chain reaction (PCR), enzyme-linked immunosorbent assay (ELISA), and combinations thereof.

6. The method of claim 1, wherein the cell membrane expression of the MEC molecule is detected by contacting the cell with an antibody targeting the MHC molecule and detecting binding between the MHC molecule and the antibody.

7. The method of claim 1, wherein the presence of tumor-infiltrating T cells in the tumor sample is detected using at least one method selected from the group consisting of immunohistochemistry, immunotluorescence, flow cytometry, mass-spectroscopy, RNA sequencing, RNA in situ hybridization, polymerase chain reaction (PCR), enzyme-linked immunosorbent assay (ELBA), and combinations thereof.

8. The method of claim 1, wherein the presence of tumor-infiltrating lymphocytes in the tumor sample is detected using Haemotoxylin and Eosin staining.

9. The method of claim 1, wherein expression of heinokine expression in the tumor sample is detected using at least one method selected from the group consisting of immunohistochemistry, immunotluorescence, flow cytometry, mass-spectroscopy, RNA sequencing, RNA in situ hybridization, polymerase chain reaction (PCR), enzyme-linked immunosorbent assay (ELISA), and combinations thereof.

10. The method of claim 1, where TP53 mutations are detected by direct sequencing.

11. The method of claim 1, and further comprising identifying the subject as likely to respond to treatment with an immunotherapeutic agent when cell membrane expression of the MHC molecule on the cell is elevated, and at least one circumstance is present, selected from the circumstances consisting of:
(i) a presence of tumor-infiltrating T cells in the tumor sample;
(ii) a presence of tumor-infiltrating lymphocytes in the tumor sample;
(iii) elevated chemokine expression in the tumor sample; and
(iv) the subject has TP53-mutation.

12. The method of claim 11, and further comprising administering a therapeutically effective amount of an immunotherapeutic agent to the subject.

13. The method of claim 12,wherein the immunotherapeutic agent is an antibody selected from anti-CTLA-4, anti-PD-L1, anti-PD-1, anti-LAGS, anti-TIM3, anti-OX40, anti-4-IBB, or an antigen-binding portion thereof.

14. The method of claim 13, and further comprising administration of a MEK, epigenetic DNA methyltransferase, or histone deacetylase inhibitor.

15. The method of claim 12, wherein the immunotherapeutic agent is administered in combination with an MDM2 antagonist or an MEK inhibitor.

16. The method of claim 15, wherein the combination comprises an anti-PD-L1 antibody and an MDM2 antagonist or an MEK inhibitor.

17. The method of claim 16, wherein the combination comprises Atezolizurnab and Cobimetinib or Idasanutlin.

18. The method of claim 1, wherein the tumor sample is formalin-fixed.

19. The method of claim 1, wherein the tumor sample is not a frozen tissue sample.

20. A method of treating cancer in a subject, comprising: administering an effective amount of a combination of (a) an antibody or an antigen-binding portion thereof that disrupts the interaction between PD-1 and PD-L1; and (b) an MDM2 antagonist or an MEK inhibitor.
